(19) United States
(12) Patent Application Publication
(10) Pub. No.: US 2026/0199630 A1
(43) Pub. Date: Jul. 16, 2026

(54) Modular Foldable Vibroacoustic Therapy System with EEG-Guided Neuromodulation and 3D Sensory Entrainment

(71) Applicant: ZENCORA NEURO HOLDINGS INC, Plano, TX (US)

(72) Inventor: Justin Nichols, Plano, TX (US)

(73) Assignee: ZENCORA NEURO HOLDINGS INC, Plano, TX (US)

(21) Appl. No.: 19/378,052

(22) Filed: Nov. 3, 2025

Related U.S. Application Data
(60) Provisional application No. 63/715,595, filed on Nov. 3, 2024.
(60) Provisional application No. 63/748,425, filed on Jan. 22, 2025.
(60) Provisional application No. 63/909,463, filed on Oct. 31, 2025.

(51) Int. Cl. A61M 21/02; A61H 9/00; A61H 23/02; A61M 21/00

(52) U.S. Cl. CPC A61M 21/02; A61H 9/0078; A61H 23/02; A61H 2201/0134; A61H 2201/10; A61H 2201/1607; A61H 2201/165; A61H 2201/5005; A61H 2201/5097; A61H 2205/022; A61H 2205/084; A61H 2230/105; A61M 2021/0022; A61M 2021/0027; A61M 2021/0044; A61M 2205/05; A61M 2205/3303; A61M 2205/3306; A61M 2205/3327; A61M 2205/3368; A61M 2205/3375; A61M 2205/3592; A61M 2205/502; A61M 2205/8206; A61M 2210/0606; A61M 2210/0687; A61M 2210/10; A61M 2230/005; A61M 2230/04; A61M 2230/10; A61M 2230/40

(57) ABSTRACT

Systems and methods for closed-loop vibroacoustic neuromodulation are disclosed. A device uses upper and lower transducer arrays to form a closed acoustic circuit that constructively couples energy through the torso; disabling the upper array under constant lower drive yields a large reduction in internal acceleration (≥10× and up to ≥50×). A latency engine preserves cross-modal phase alignment using per-user delay profiles and sub-second to sub-millisecond compensation. Group sessions are enabled by a synchronization server maintaining phase-coherent outputs across nodes. A craniofacial “Beauty Bar” docks into textile accessories (weighted throws, cushions, pillow inserts) to serve as an over-body array, with offline SD operation and a back-to-sleep control. Thermal safety employs a dual-exponential predictive model with redundant sensors, watchdog, and analog cutoff. Retail kiosk analytics acquire brief EEG/biometric data to generate personalized profiles and to provision portable devices for home use.

### CROSS-REFERENCE TO RELATED APPLICATIONS

This application claims the benefit of U.S. Provisional Application Nos. 63/715,595, 63/748,425, and No. 63/909,463, which are hereby incorporated by reference in their entirety.

### FIELD OF THE INVENTION

This disclosure relates to vibroacoustic therapy systems and multi-modal neuromodulation, and more particularly to foldable, modular vibroacoustic systems integrated with HD EEG-based adaptive control, multi-layer transducer configurations, and real-time sensory synchronization for enhanced personalized therapy.

### BACKGROUND OF THE INVENTION

Vibroacoustic therapy, which delivers low-frequency sound vibrations to the body for therapeutic effects, has been used in various contexts to promote relaxation, enhance circulation, and support neurological well-being. More recently, multi-sensory entrainment approaches have attempted to synchronize auditory, visual, and tactile stimuli to modulate neural activity and optimize cognitive outcomes. Systems like GENUS (Gamma Entrainment Using Sensory Stimuli) and certain commercial vibroacoustic beds demonstrate the potential of these methods.

However, existing systems remain limited in key areas: Lack of Portability and Modularity. Most current vibroacoustic platforms are large, fixed beds or tables with minimal adaptability. These systems are not optimized for home use, group settings, or mobile deployment. They also lack the flexibility to target specific body regions or adapt to different therapeutic environments. Insufficient Sensory Immersion. Traditional systems deliver vibrations primarily from beneath the user. This unidirectional stimulation does not fully engage the body's three-dimensional sensory network, especially in high-innervation zones such as the hands, spine, and midline torso. Inadequate Personalization. Many systems lack integration with real-time biometric or neural feedback mechanisms. They apply generic protocols rather than adjusting stimulation based on individual EEG signatures, sleep stage, or other physiological data. This limits their therapeutic precision and effectiveness. Weak Synchronization Across Modalities. Optimal neuromodulation depends on the precise timing of multi-sensory stimuli. Latency mismatches between audio, tactile, and visual channels can diminish the brain's capacity for entrainment. Existing products rarely address real-time phase alignment, let alone compensate for individual transmission delays or processing differences. Limited Scalability for Group Therapy or Clinical Deployment. Current systems are generally designed for solo use. There is minimal support for synchronized group sessions, shared protocols, or biometric-driven feedback loops in multi-user contexts.

There remains an unmet need for a highly portable, modular, and intelligent vibroacoustic therapy system that: Delivers 3D vibrational stimulation via repositionable, multi-layer transducer arrays. Interfaces with HD EEG or biometric sensors to enable real-time neural feedback and adaptation. Utilizes redundant synchronization mechanisms (e.g., photocells, microphones, accelerometers) to ensure phase-accurate sensory input. Supports group therapy, sleep ecosystem integration, and optional companion modules for pets or environmental inputs. Operates flexibly in home, clinical, or mobile settings, offering scalable therapy options for diverse user profiles.

### SUMMARY OF THE INVENTION

The present disclosure relates to systems and methods for adaptive neuromodulation employing multi-modal sensory and electrophysiological feedback to dynamically regulate neural activity in real time. More particularly, the disclosure provides an integrated stimulation architecture that combines vibroacoustic, photonic, and transcranial electrical modalities with adaptive control algorithms and robotic positioning subsystems to deliver personalized, closed-loop neuromodulation through craniofacial bone-conductive pathways.

In one aspect, the disclosure comprises a multi-layer stimulation assembly configured to deliver at least two stimulation modalities selected from: (a) vibroacoustic stimulation, (b) photonic (light-based) stimulation, and (c) transcranial alternating-current stimulation (tACS). The assembly is operatively positioned adjacent to the user's cranial or cervical region, enabling multimodal coupling to neural substrates associated with cortical and subcortical networks.

The system further includes a craniofacial bone-conduction interface, such as a bar or frame mechanically coupled to one or more of the maxilla, mandible, zygomatic arch, or mastoid region. This interface houses one or more vibroacoustic transducers or photonic emitters that transmit energy directly through craniofacial bone structures, thereby achieving efficient mechanical and optical coupling without invasive contact or conductive gels.

To maintain optimal positioning and consistent coupling efficiency, the system incorporates robotic actuators comprising multi-degree-of-freedom arms capable of precisely orienting, positioning, and applying calibrated contact force to the stimulation assembly. These actuators can automatically adapt to individual anatomical differences or dynamic user movements, ensuring stable and repeatable stimulation geometry.

Neural feedback is obtained through a neural signal acquisition unit, which includes at least one high-density electroencephalography (HD-EEG) sensor array configured to record electrophysiological signals from the user in real time. The recorded neural data are continuously analyzed by an onboard or networked computing system that executes adaptive algorithms for personalization and closed-loop control.

The computing system comprises:
(a) a personalization algorithm configured to adapt stimulation parameters based on real-time EEG data and stored user response profiles;
(b) memory for storing EEG data, stimulation settings, and user metadata; and
(c) a processing engine executing reinforcement-learning, Bayesian-optimization, or other adaptive control techniques that iteratively refine stimulation delivery to optimize neural entrainment and therapeutic outcome.

A control interface provides operator or automated oversight of the closed-loop system, enabling continuous modulation of stimulation output in response to the user's changing electrophysiological state. The system dynamically adjusts parameters such as frequency, amplitude, phase, and waveform type to maintain targeted neural synchronization or to promote specific neuromodulatory effects.

Through the integration of multi-modal stimulation, craniofacial bone-conduction pathways, robotic positioning, and adaptive EEG-based feedback, the disclosure provides a self-calibrating neuromodulation platform capable of real-time personalization. The architecture supports both therapeutic and research applications, enabling precise, repeatable, and intelligent modulation of brain activity without invasive intervention.

### BRIEF DESCRIPTION OF THE DRAWINGS

FIG. 1 depicts a vibroacoustic neuromodulation system in accordance with an embodiment.

FIG. 2 depicts a control and feedback architecture, in accordance with an embodiment.

FIG. 3 depicts a multi-transducer vibroacoustic neuromodulation platform, in accordance with an embodiment.

FIG. 4 depicts a brain network mapping and optimization process, in accordance with an embodiment.

FIG. 5 depicts a vibroacoustic neuromodulation system, in accordance with an embodiment.

FIG. 6 depicts a multi-layer topper assembly, in accordance with an embodiment.

FIG. 7 depicts a modular vibroacoustic neuromodulation system, in accordance with an embodiment.

FIG. 8 depicts a vibroacoustic neuromodulation system, in accordance with an embodiment.

FIG. 9 depicts vibroacoustic transducer module assemblies, in accordance with an embodiment.

FIG. 10 depicts a multi-user synchronization and group neuromodulation architecture, in accordance with an embodiment.

FIG. 11 depicts an adaptive calibration and neuroresponse tracking system, in accordance with an embodiment.

FIG. 12 depicts a model-based neuromodulation control and analytics system, in accordance with an embodiment.

FIG. 13 depicts a distributed neuromodulation ecosystem, in accordance with an embodiment.

FIG. 14 depicts a thermal-safety and closed-loop control architecture, in accordance with an embodiment.

FIG. 15 depicts a vibration monitoring and amplitude control subsystem, in accordance with an embodiment.

FIG. 16 depicts a user-mounted vibration monitoring and amplitude calibration subsystem, in accordance with an embodiment.

FIG. 17 depicts an EEG- or hdEEG-integrated feedback control subsystem, in accordance with an embodiment.

FIG. 18 depicts a behavioral psychophysics and gamma optimization subsystem, in accordance with an embodiment.

FIG. 19 depicts a unified multi-modal control architecture, in accordance with an embodiment.

### DETAILED DESCRIPTION OF THE INVENTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents. The present application describes several embodiments, and none of the statements below should be taken as limiting the claims generally.

Where block diagrams have been used to illustrate the embodiments, it should be recognized that the physical location where described functions are performed are not necessarily represented by the blocks. Part of a function may be performed in one location while another part of the same function is performed at a distinct location. Multiple functions may be performed at the same location. In a functional block diagram, a single line may represent a connection, in general, or a communicable connection, particularly in the presence of a double line, which may represent a power connection. In either case, a connection may be tangible, as in a wire, or radiated, as in near-field communication. An arrow may typically represent the direction of communication or power although should not be taken as limiting the direction of connected flow.

### Overview of the System Architecture

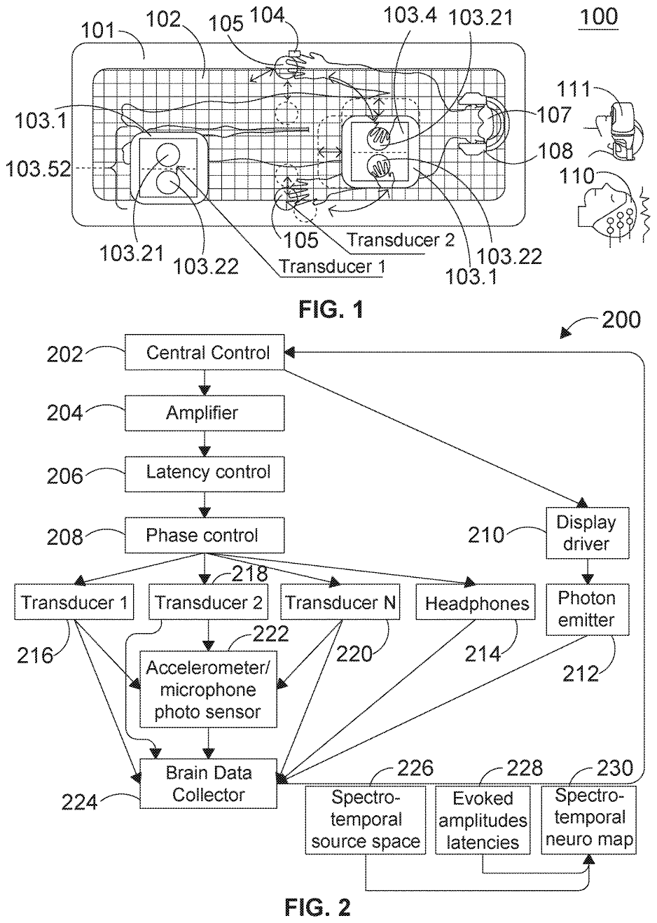

FIG. 1 illustrates an exemplary embodiment of a vibroacoustic neuromodulation system (100) configured to deliver coordinated multi-modal sensory stimulation to a user positioned upon a modular therapy surface (101). The system (100) comprises a base platform or table structure (101) supporting a topper module (102) that houses a plurality of modular vibroacoustic transducer assemblies (103.1, 103.21, 103.22). Each transducer assembly is configured to deliver mechanical vibrational energy to selected regions of the user's body, as shown schematically by Transducer 1 and Transducer 2 in the figure.

The topper (102) may include internal cavities, pockets, or compliant recesses dimensioned to receive the transducer modules (103.1-103.22) in interchangeable configurations. In certain embodiments, the transducer modules comprise soft cushion housings (103.1) enclosing one or more transducer pucks (105) and optional backer boards (103.4). The transducer assemblies may be oriented in either puck-in or puck-out configurations to tailor vibrational coupling and perceived intensity, with puck-out arrangements (e.g., 103.52) delivering concentrated energy upward toward the user and puck-in configurations providing diffused, gentle stimulation. Multiple transducer units can operate in phase, out of phase, or independently to produce spatialized or dynamic waveforms across the body.

A physiological sensor module (104) is positioned on or near the user to capture biometric signals such as heart rate, galvanic skin response, respiration, or motion. The sensor (104) communicates with the control subsystem (107, 110) to enable real-time monitoring and adaptive modulation of the stimulus parameters. The vibroacoustic transducer pucks (105) may be positioned along the torso, extremities, or other target regions to deliver localized mechanical energy synchronized with auditory or visual stimuli.

A light-shield (107) may be placed over the user's head to minimize ambient distraction and assist in visual entrainment protocols. Headphones (108) deliver synchronized auditory output, such as binaural beats or modulated sound fields, coordinated temporally with the vibroacoustic patterns. Together, the tactile, auditory, and visual stimuli create a unified multi-sensory experience designed to induce neural entrainment and promote relaxation, focus, or neuroplastic adaptation.

The control unit (110) incorporates signal-generation electronics, including amplifiers, latency-control modules, and phase-synchronization circuits, which govern the output waveforms delivered to each transducer (103) and to the auditory and photonic channels. In certain embodiments, the control unit (110) communicates wirelessly or via a tethered interface with an external user interface module (111), such as a tablet or smart device, allowing a practitioner or user to select session parameters, initiate calibration routines, and monitor biometric feedback in real time.

During operation, the system (100) generates a closed feedback loop between the user's physiological state and the vibroacoustic stimulus output. Data captured by the physiological sensors (104) are analyzed by the control unit (110), which dynamically adjusts amplitude, frequency, and phase relationships among the transducers (103.1, 103.21, 103.22) and associated auditory and visual channels. The resulting closed-loop configuration enhances therapeutic precision, compensates for latency mismatches, and maintains synchronization across modalities throughout the session.

Accordingly, FIG. 1 demonstrates how the disclosed vibroacoustic neuromodulation system integrates multi-modal stimulation, real-time biometric sensing, and adaptive control within a modular and reconfigurable therapeutic platform suitable for both clinical and personal wellness applications.

FIG. 2 illustrates a schematic block diagram of a control and feedback architecture (200) for use with the vibroacoustic neuromodulation system previously shown in FIG. 1. The architecture (200) governs the generation, synchronization, and adaptive modulation of multi-modal stimuli, including vibroacoustic, auditory, and photonic outputs.

The control architecture (200) comprises a central control unit (202) operatively coupled to an amplifier stage (204). The amplifier (204) provides voltage and current drive signals to a plurality of transducer channels and associated sensory modules under timing and amplitude constraints defined by the central control logic. A latency-control subsystem (206) and a phase-control subsystem (208) are integrated within the control path to ensure temporal coherence and precise phase alignment among concurrently delivered stimuli.

The control unit (202) communicates with one or more vibroacoustic transducer assemblies (216, 218, 220), each corresponding to discrete zones or channels on the therapy platform. In certain embodiments, the transducers correspond to the modules (103.21, 103.22) described in FIG. 1. Each transducer receives a waveform derived from the amplifier (204) and is dynamically modulated according to feedback from sensor inputs and the adaptive algorithms executed by the control unit.

Associated with the transducer array are biometric and environmental sensors (222), which may include accelerometers, microphones, and photodiodes to capture motion, vibration amplitude, and ambient light levels during operation. These sensors transmit data to a brain data collector (224), which performs real-time signal conditioning and feature extraction, such as artifact removal, frequency decomposition, and synchronization tagging relative to stimulus onset.

The control architecture further includes additional sensory output channels. A display driver (210) and a photon emitter (212) may be provided to deliver temporally coordinated visual stimulation (e.g., flicker, color, or intensity modulation). Similarly, headphones (214) or other auditory transducers provide synchronized acoustic output aligned in time and phase with the vibroacoustic signals.

The processed feedback data from the brain data collector (224) are analyzed to generate or update one or more spectro-temporal models, including a spectro-temporal source space (226), an evoked amplitude and latency map (228), and a spectro-temporal neuro-map (230). These models represent instantaneous and averaged neural responses to stimulation, enabling the control system to refine the drive signals adaptively based on user-specific biomarkers.

The spectro-temporal data are communicated back to the latency and phase-control modules (206, 208), forming a closed adaptive feedback loop that continuously adjusts stimulus parameters. Through this feedback mechanism, the system (200) maintains real-time synchronization between neural activity and multimodal stimulation, compensating for user variability, physiological drift, and device latency.

Accordingly, FIG. 2 illustrates the functional organization of the control, sensing, and feedback subsystems of the vibroacoustic neuromodulation platform, wherein distributed transducer nodes (216-220) operate in coordination with auditory and visual outputs under real-time, closed-loop adaptive control governed by physiological feedback and neural response data.

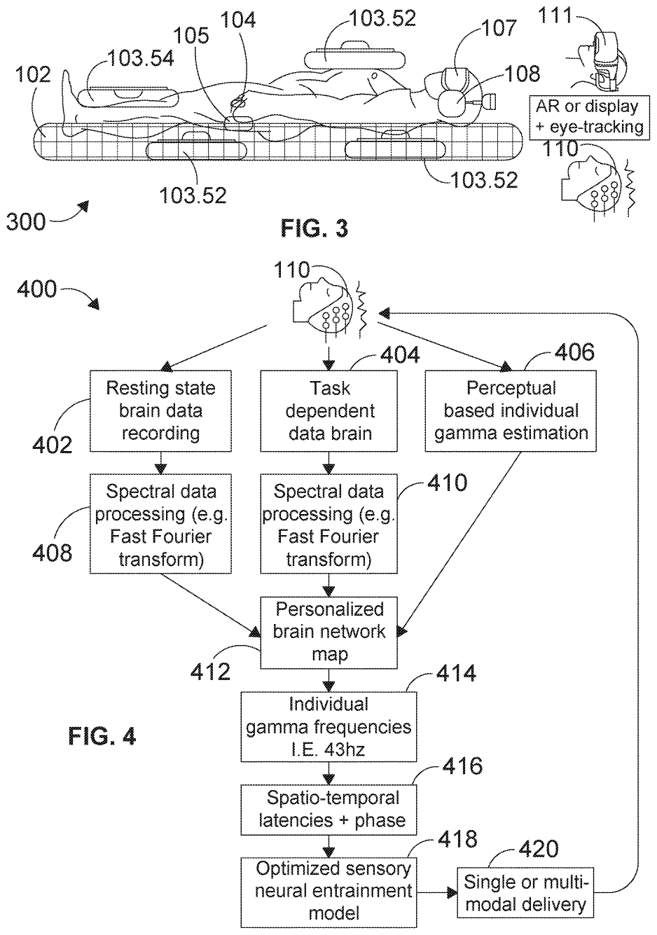

FIG. 3 illustrates, in side-sectional view, an embodiment of a multi-transducer vibroacoustic neuromodulation platform (300) configured to deliver synchronized, multi-channel vibrational stimulation to a user positioned on the modular topper surface (102). The system incorporates both under-body and over-body transducer assemblies (103.52, 103.54) to create a bilateral vibroacoustic field encompassing the user.

The base or support structure (101) houses a plurality of modular transducer cushions (103.1), each containing one or more vibroacoustic transducer elements (105). These elements are positioned at selected anatomical locations to correspond to the user's torso, legs, and cranial regions, enabling targeted mechanical stimulation of different somatic and cortical representations. Each transducer assembly may include an internal backer or mounting plate (103.4) configured to direct acoustic energy upward into the user's body in a controlled manner. The puck-out configuration (103.52) illustrated beneath the torso and legs delivers high-intensity vibrational energy directed toward the user, while the puck-in configuration may be used for gentler, diffused stimulation.

In the embodiment shown, an upper transducer module (103.54) is positioned above the user to provide complementary over-body stimulation, producing constructive interference patterns between the upper and lower arrays. The resulting acoustic coupling forms a three-dimensional vibroacoustic envelope that modulates both tactile and proprioceptive pathways to support neural entrainment.

A physiological sensor array (104) is positioned in contact with or proximate to the user's body to monitor biometric variables such as heart rate, galvanic skin response, respiration, and local accelerometric motion. These sensor data are transmitted in real time to the control unit (110), which also receives neural and cognitive input from a head-mounted system (107, 108, 111). The head-mounted system may comprise auditory output transducers (headphones 108) and a visual interface such as an augmented-reality or display unit (107, 111) optionally incorporating eye-tracking sensors to monitor gaze or pupil response.

The control unit (110) executes latency-mapping and phase-synchronization algorithms to align vibroacoustic output (from transducers 103.52 and 103.54) with concurrent auditory and visual stimuli. This synchronization enables precise multi-modal stimulation, where frequency and phase of mechanical, acoustic, and photonic energy are modulated according to the user's detected physiological state. The adaptive feedback loop thereby ensures that stimulation parameters are continuously adjusted to maintain resonance within user-specific neural frequency bands identified through the feedback data.

During operation, the user experiences coordinated vibration, sound, and visual entrainment patterns generated by the distributed transducer assemblies and peripheral sensory modules. The integration of upper and lower transducer pairs (103.52 and 103.54) with the sensing and control system (104, 110) provides a dynamically tuned closed-loop neuromodulation environment, capable of reinforcing neural oscillations, reducing stress, or promoting therapeutic plasticity.

Accordingly, FIG. 3 demonstrates how the disclosed neuromodulation system physically implements multi-point vibroacoustic stimulation, real-time biometric feedback, and adaptive synchronization between body-coupled and sensory channels to form a responsive, full-body neuromodulatory interface.

FIG. 4 illustrates a schematic block diagram of a brain network mapping and optimization process (400) implemented by the control and analytics architecture of the vibroacoustic neuromodulation system. The process (400) enables the derivation of individualized stimulation parameters based on resting-state and task-dependent neural recordings, yielding a personalized sensory entrainment model used to drive adaptive multimodal therapy delivery.

In the depicted embodiment, neural data are first obtained via one or more electrophysiological acquisition devices (110), such as a high-density EEG subsystem described previously in connection with FIGS. 1-3. The system records both resting-state brain data (402) and task-dependent brain data (404), which collectively capture baseline and dynamic neural activity across multiple cognitive and sensory domains.

In certain embodiments, perceptual calibration data are also obtained through a perceptual-based individual gamma estimation module (406). This step involves visual or auditory paradigms that assess each user's intrinsic oscillatory resonance frequencies—such as gamma, alpha, or theta bands—to personalize frequency tuning of the entrainment protocol.

The acquired neural data (402, 404, 406) are then subjected to spectral data processing (408, 410), for example through fast Fourier transform (FFT) or equivalent signal-decomposition techniques. These algorithms extract temporal, frequency, and coherence features across cortical regions to quantify network connectivity, phase relationships, and amplitude envelopes.

Processed data from each pathway converge within a personalized brain network mapping module (412). This module generates a multi-dimensional representation of the user's neural connectivity, identifying dominant oscillatory frequencies and spatial-temporal coherence patterns unique to the individual. From this personalized map (412), the control system derives one or more individual gamma frequencies (414)—for instance, approximately 43 Hz in certain embodiments-representing the optimal target for neural entrainment or modulation.

Subsequently, a spatio-temporal latency and phase-analysis subsystem (416) characterizes the timing offsets and propagation delays between sensory inputs and cortical responses. These latency parameters inform synchronization adjustments within the stimulation delivery pipeline, ensuring that vibroacoustic, auditory, and visual signals arrive at the user's sensory interfaces in precise phase alignment.

Based on the extracted frequency and timing data, the system generates an optimized sensory neural entrainment model (418). This model defines amplitude, frequency, and phase relationships for the various sensory channels to achieve coherent neural resonance across modalities.

Finally, the optimized entrainment model is transmitted to a stimulus delivery controller (420), which executes either single-modality or multi-modality stimulation according to the individualized parameters. The resulting delivery may involve vibroacoustic, auditory, visual, or combined stimuli modulated to reinforce user-specific neural oscillatory patterns identified earlier in the process.

Through this adaptive analytical loop, the process (400) allows the neuromodulation system to evolve from generalized stimulation protocols to a highly personalized, closed-loop entrainment architecture, continuously refined through ongoing neural data acquisition and analysis.

Accordingly, FIG. 4 demonstrates the computational framework through which the disclosed system transforms raw neural and perceptual data into individualized models for synchronized multimodal neuromodulation, thereby enhancing therapeutic precision and reproducibility across users.

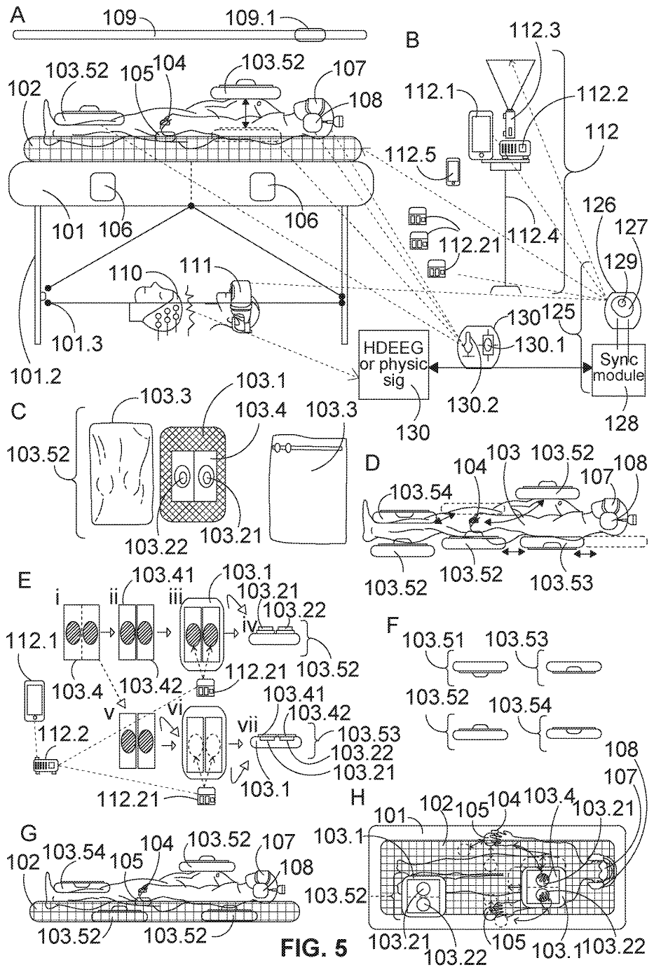

FIG. 5 illustrates multiple views and operational configurations of a vibroacoustic neuromodulation system (100) according to representative embodiments. The figure collectively depicts the physical arrangement of modular transducers, sensor subsystems, control interfaces, and example deployment modes for single-user or networked operation.
View A—System Overview (Full Assembly). In view A, a user is shown positioned upon the support platform (101) and topper assembly (102). Embedded within the topper are multiple modular transducer cushions (103.1, 103.21, 103.22) containing individual vibroacoustic transducer elements (105) configured to deliver mechanical energy upward toward the user. Select transducers are arranged in puck-out configurations (103.52) to provide high-intensity localized vibration, while other regions may utilize puck-in configurations for diffuse stimulation.

A sensor array (104) positioned adjacent the user monitors biometric and motion parameters. The control unit (110) communicates with an external interface module (111) and an augmented-reality or display apparatus (107, 108) that provides synchronized visual and auditory stimulation. A handheld sensor wand (101.3) or electrode assembly may connect to the control unit for optional physiological data collection. An elongated upper transducer bar (109, 109.1) positioned above the user may be used for over-body stimulation, forming a bilateral acoustic field between the upper and lower transducer arrays (103.52, 103.54).
View B—Control and Network Connectivity. View B shows the electronic and communication subsystem (112) that interconnects the user platform with external sensors and computing modules. The system may include one or more mobile devices or tablets (112.1, 112.2) executing software for waveform generation, calibration, and data logging. A wireless communication interface (112.3) provides connectivity between the local control node (125) and a synchronization box or network bridge (128) for multi-device coordination. Auxiliary submodules (112.4, 112.5) represent optional cloud, EEG, or physiological data interfaces (130), enabling distributed or cloud-based signal analysis and adaptive control.

Power delivery and signal routing components (125-130) coordinate operation of the vibroacoustic, auditory, and photonic channels under the direction of the central controller. The architecture supports both standalone and group-therapy synchronization modes via the sync box (128).
View C—Modular Cushion Architecture. View C provides enlarged detail of the modular cushion assemblies (103.1-103.3). Each cushion comprises a soft enclosure (103.1) containing one or more transducer pucks (103.21, 103.22) mounted on a backer board (103.4) and optionally sealed by a zipper or access flap (103.3) to permit replacement, cleaning, or reconfiguration. The configuration enables interchange of different transducer types or orientations (puck-in, puck-out) to vary vibrational intensity, frequency response, and mechanical impedance.
View D—User Interaction in Closed-Loop Mode. View D illustrates the user during an active stimulation session. Transducers (103.52, 103.53, 103.54) operate in complementary patterns along the body, coordinated with the sensor module (104) and head-mounted display and headphone assembly (107, 108). The control unit (110) dynamically adjusts the phase and amplitude of vibroacoustic signals in real time according to the biometric data stream, implementing the closed-loop neuromodulation protocol previously described with respect to FIGS. 2 and 3.
View E—Transducer Orientation and Assembly States. View E (sub-views i-vii) demonstrates alternative transducer module orientations and reconfigurable assemblies. The sequence depicts insertion and rotation of transducer elements (103.4, 103.41, 103.42) within cushion housings (103.1), forming either puck-in or puck-out modes (103.51-103.54). These orientations allow directional control of vibrational energy and enable the user or clinician to fine-tune coupling strength to the body. The illustrated configurations also support paired transducer alignment, cross-phase operation, and stereo vibroacoustic synthesis, where two transducers function analogously to left- and right-channel speakers in synchronized phase opposition.

Associated interface devices (112.1-112.4) are shown transmitting configuration data to the controller, enabling automated recognition of module orientation via embedded RFID, accelerometer, or impedance-based detection systems (112.21, 112.22).
View F—Cross-Sectional Transducer Modes. View F depicts simplified cross-sections of multiple transducer configurations (103.51-103.54). These examples highlight differing mechanical coupling geometries and energy propagation paths through the mat and body interface. In some embodiments, opposing transducers are driven in phase to create constructive interference zones; in others, alternating phase drive patterns are used to generate traveling or pulsatile waves across the surface.
View G—Bilateral Acoustic Coupling Arrangement. View G demonstrates a bilateral acoustic coupling configuration wherein lower transducer elements (103.52) are paired with corresponding upper modules (103.54). This arrangement forms a closed acoustic circuit around the user's body, enhancing signal penetration and allowing modulation of amplitude nodes and pressure gradients to achieve desired entrainment effects.
View H—Top Plan View of Operational Layout. View H provides a top plan view summarizing the complete operational layout of the system, showing the placement of transducer assemblies (103.1-103.22), sensor module (104), and head-mounted interface (107, 108). The view illustrates representative signal paths and control links among the physical components during a session, emphasizing the distributed, modular nature of the system architecture.

Summary: Collectively, FIG. 5 depicts multiple embodiments and sub-assemblies of the disclosed vibroacoustic neuromodulation system, demonstrating how modular transducer cushions, over-body arrays, sensor networks, and control electronics are combined to form a configurable, closed-loop therapeutic platform. The illustrated variants show the adaptability of the design to different use cases—ranging from single-user relaxation to clinical neuromodulation research—and support various transducer geometries, wireless configurations, and synchronized multimodal feedback protocols.

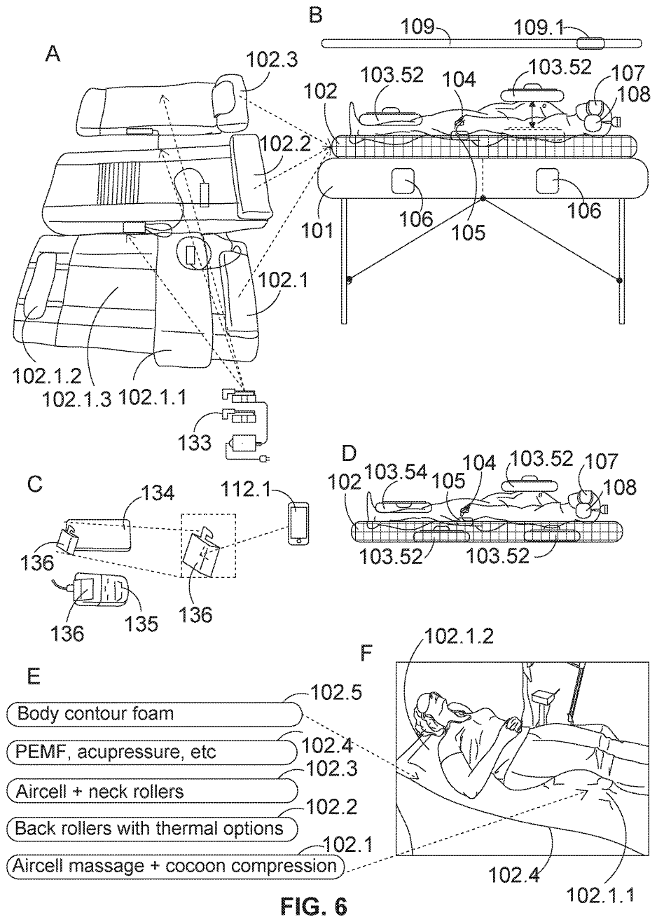

FIG. 6 illustrates various embodiments and configurations of a multi-layer topper assembly (102) forming part of the vibroacoustic neuromodulation system described previously. The topper (102) provides both mechanical support and modular therapeutic functionality, integrating vibroacoustic, pneumatic, thermal, and electromagnetic stimulation layers within a reconfigurable structure.
View A—Exploded Topper Configuration. In view A, the topper assembly (102) is shown in exploded form, comprising multiple stacked and separable layers (102.1-102.3). The base layer (102.1) forms the structural foundation of the topper and may include internal air cells, pneumatic chambers, or embedded massage mechanisms (102.1.1, 102.1.2). These components provide aircell compression, cocooning, or contouring functionality, adjustable via an external control unit (133).

Above the base layer (102.1) resides a roller or thermal layer (102.2), which can include mechanical rollers configured to traverse the surface along one or more axes, optionally providing heat or vibration through embedded transducers (105) or resistive elements. A neck and shoulder therapy layer (102.3) may include integrated aircell actuators, vibroacoustic pucks, or electromagnetic modules for localized stimulation of the cervical region.

In certain embodiments, each layer (102.1-102.3) connects electrically or pneumatically to a common controller via detachable connectors or internal routing channels, allowing modular reconfiguration, replacement, or servicing. The topper assembly (102) mounts atop the support platform (101), as shown in FIG. 5, to form a unified therapeutic surface.
View B—System Integration. View B depicts the topper (102) integrated onto the base platform (101), in conjunction with transducer assemblies (103.52), sensor module (104), and the head-mounted display and auditory interface (107, 108). The upper transducer bar (109, 109.1) is again shown above the user, providing over-body vibroacoustic or photonic stimulation in conjunction with the under-body transducer array.

This view demonstrates how the topper layers (102.1-102.3) cooperate with the transducer and control elements to deliver coordinated sensory stimulation. Pneumatic, vibrational, and thermal stimuli may be synchronized via the control unit to match specific neural frequencies or entrainment targets identified through real-time feedback.
View C—Pneumatic Control and Aircell System. View C provides an enlarged depiction of the pneumatic control subsystem. A portable controller module (133) communicates via a wired or wireless interface (112.1) with internal air pumps (134), valves, or actuators (135, 136). The system allows selective inflation or deflation of aircell segments (102.1.1, 102.1.2) to achieve cocoon compression or adaptive body contouring.

In certain embodiments, the pneumatic controller (133) may also regulate vibration or heating elements embedded in the same layer, enabling compound sensory effects such as synchronized compression and vibration in phase with the vibroacoustic drive waveform.
View D—Coupled Vibroacoustic and Pneumatic Therapy. View D illustrates a user positioned upon the topper assembly (102) during an active session, wherein lower transducer elements (103.52) and upper elements (103.54) operate in coordination with the pneumatic chambers (102.1-102.2). The feedback sensor (104) monitors physiological signals while the auditory and visual modules (107, 108) deliver synchronized stimulation.

In this mode, the pneumatic pressure cycles and vibroacoustic output are phase-locked by the control unit to create rhythmic entrainment patterns, enhancing user relaxation or targeted neuromodulation.
View E—Functional Layer Enumeration. View E enumerates the principal functional layers of the topper assembly (102):

| Layer | Description |
|---|---|
| 102.1 | Aircell massage and cocoon compression layer |
| 102.2 | Back rollers with thermal options |
| 102.3 | Aircell + neck roller module |
| 102.4 | PEMF/acupressure/secondary therapy layer |
| 102.5 | Body contour foam or surface comfort layer |

Each layer can operate independently or in synchrony under control of the main system logic. Signals from the vibroacoustic control module, pneumatic subsystem, and user interface are routed through a multi-channel controller (133), allowing programmable sequences or manual overrides.
View F—Active Use Case. View F depicts a user engaged in a therapy session on the topper assembly (102), showing the integration of pneumatic compression zones (102.1-102.2) with localized acupressure or PEMF stimulation (102.4). The topper conforms dynamically to the user's body, providing customized pressure distribution while maintaining alignment with the vibroacoustic transducers positioned beneath the surface. The user may operate or monitor the system using a handheld device or mobile interface (112.1) linked wirelessly to the control electronics.

Collectively, FIG. 6 illustrates the mechanical and functional architecture of the modular topper assembly, demonstrating how pneumatic, thermal, electromagnetic, and vibroacoustic modalities are integrated into a unified therapeutic platform. The configuration allows individual or combined operation of layers, enabling customizable sensory profiles and precise neuromodulatory targeting in coordination with the system's adaptive control framework.

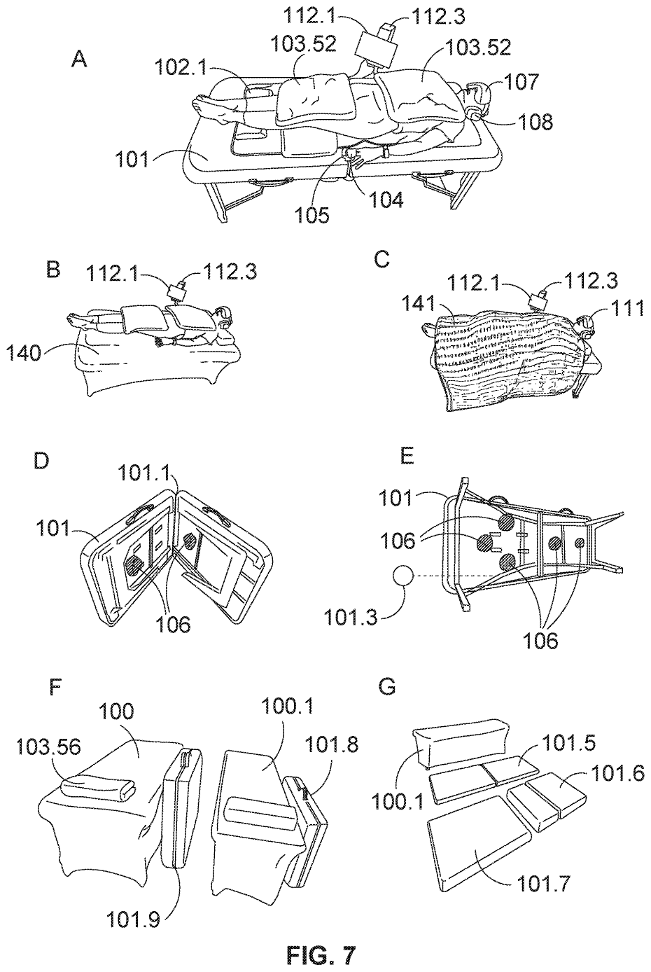

FIG. 7 illustrates various embodiments and configurations of the modular vibroacoustic neuromodulation system (100), showing portable, foldable, and customizable arrangements as well as calibration and coupling examples. The system (100) may be configured for clinical, research, or consumer use, with detachable modules, integrated transducers, and wireless control capability.
View A—Full System Configuration (Calibration Mode). In view A, a user is shown positioned on the support platform (101) with the multi-layer topper assembly (102.1) placed atop the surface. A plurality of transducer modules (103.52) are arranged along the user's body, and a sensor array (104) captures real-time biometric and vibroacoustic feedback signals. The system communicates wirelessly with an external calibration and control interface (112.1, 112.3), which may be a mobile device or tablet executing the system's configuration software.

Auditory output is delivered via headphones (108), while visual synchronization or augmented-reality display may be provided through the head-mounted interface (107). The calibration process involves automatic tuning of phase, amplitude, and timing of the individual transducer outputs (105) based on measured resonance and latency feedback obtained through the sensors (104) and internal microphones embedded in the mat structure.
View B—Compact or Clinical Bench Configuration. View B shows an alternative compact form factor (140) suitable for use as a bench or reclined chair-based embodiment, wherein the vibroacoustic topper (102.1) and selected transducer modules (103.52) are integrated into a shorter platform for seated therapy or partial-body neuromodulation. Wireless connectivity via the control module (112.1, 112.3) permits the same adaptive feedback and synchronization functionality as in the full-length embodiment.
View C—Portable Relaxation or Sleep Entrainment Mode. View C illustrates a portable implementation of the system wherein a user rests on a soft flexible platform or topper (141), which incorporates compact transducer elements and a light mesh cover. The control module (112.1) and interface display (111) are wirelessly linked, enabling the user to initiate preset therapy programs. This embodiment may be powered by battery or USB interface, suitable for home use, meditation, or travel.
Views D and E—Foldable Platform Structure and Internal Frame. Views D and E depict the foldable support platform (101) used to house and align the internal transducer mounting bays (106). In view D, the platform is shown partially folded, illustrating hinge mechanisms (101.1) and modular cavities that receive individual transducer assemblies (105, 106). In view E, the internal structural frame is shown with multiple mounting recesses and wiring channels (101.3) configured to route signal and power lines between the embedded components and the main controller.

The foldable design enables compact storage and portability without compromising mechanical coupling between the transducers and the topper assembly. When deployed, the segments unfold to a planar alignment with secure interlocking, ensuring consistent vibrational transmission across the surface.
View F—Alternative Cushion and Transducer Housings. View F illustrates a series of detachable transducer housings (100, 100.1) and padded cushion elements (101.8, 101.9) incorporating integrated vibroacoustic actuators (103.56). These modules may be attached to or removed from the main system using mechanical latches or magnetic connectors, allowing the user or clinician to customize transducer count, location, and orientation according to the desired therapy protocol.

In some embodiments, these portable housings can be used independently as localized vibroacoustic applicators, for example on the lower back or neck, and synchronized wirelessly to the central controller via Bluetooth or other communication standards.
View G—Modular Panel Construction. View G shows an exploded or disassembled view of the modular platform panels (101.5-101.7) comprising the primary structure. Each panel (101.5, 101.6, 101.7) contains pre-formed cavities and coupling hardware for receiving transducer pucks or pneumatic modules. When assembled, the panels interconnect mechanically and electrically through internal bus connectors, enabling rapid reconfiguration or repair.

This modular panel design allows the system to scale in size—from a compact single-user pad to a full clinical treatment bed—while maintaining signal integrity and synchronization across transducer channels.

Collectively, FIG. 7 demonstrates the versatility of the disclosed modular vibroacoustic neuromodulation platform across multiple embodiments, including full-length, compact, and portable configurations. The figure further illustrates how transducer arrays, toppers, and control systems may be arranged for calibration, maintenance, and transport. The foldable and modular construction enables cost-effective manufacturing, field calibration, and scalable deployment, while maintaining the closed-loop adaptive feedback and multi-modal stimulation capabilities described in preceding figures.

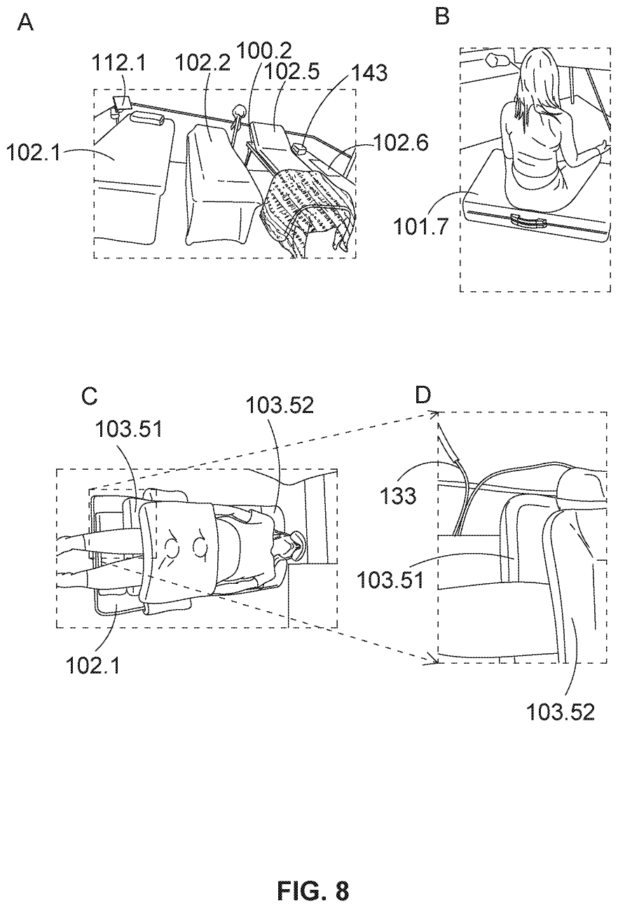

FIG. 8 illustrates additional embodiments of the vibroacoustic neuromodulation system (100) configured for compact, portable, or localized applications. The figure emphasizes how modular topper assemblies (102) and transducer elements (103) may be combined in different geometries to deliver targeted neuromodulatory stimulation under adaptive control.
View A—Modular Topper Array and Layer Composition. View A depicts a modular arrangement of topper units (102.1-102.6) positioned adjacent to one another to form a multi-segment vibroacoustic array. Each segment may correspond to a specific body region (e.g., lumbar, thoracic, or cervical) and may contain embedded transducer assemblies (103.52) or pneumatic cells. The intermediate topper modules (102.2, 102.5) may include thermal or electromagnetic layers, while the end modules (102.1, 102.6) incorporate contour foam or aircell compression zones for stability and comfort.

A controller interface (112.1) communicates wirelessly with the array, enabling independent or synchronized activation of each topper segment. In certain embodiments, an upper body module (100.2) and mesh overlay (143) provide supplemental tactile feedback or compression for relaxation and proprioceptive reinforcement.
View B—Compact Seating or Meditation Configuration. View B illustrates a seated embodiment in which the user rests upon a compact platform (101.7) containing a simplified transducer array beneath a topper segment (102.6). This configuration allows delivery of localized vibration to the lumbar or sacral regions, suitable for meditation, postural therapy, or stress reduction.

The compact form factor may be battery-powered and controlled via a mobile device or integrated touch panel. The same transducer architecture and adaptive feedback algorithms described in earlier figures are retained, providing continuity of neuromodulatory effect across seated or supine use cases.
View C—Targeted Neuromodulation Configuration. View C shows a user lying on a modular topper (102.1) equipped with paired vibroacoustic transducer elements (103.51, 103.52). These transducers are positioned beneath specific anatomical zones—for example, the lower back and upper thoracic regions—to deliver binaural or bilateral vibration. The system can modulate phase and amplitude across transducers to generate standing wave fields or pulsatile entrainment rhythms along the body's longitudinal axis.

The arrangement may also serve for experimental or therapeutic protocols requiring spatial control of vibrotactile fields, allowing researchers or clinicians to evaluate sensory coupling or somatic resonance phenomena.
View D—Detailed View of Transducer Assembly and Controller Integration. View D provides an enlarged view of the transducer modules (103.51, 103.52) and their connection to the pneumatic and control subsystem (133). Each transducer assembly may contain multiple drivers mounted on flexible plates, encased within a compliant housing for comfort and consistent body contact. The control unit (133) regulates the frequency, phase, and duty cycle of each element according to the programmed therapy profile or the user's real-time biometric feedback.

The system may employ differential drive signals between the transducers (103.51, 103.52) to establish alternating phase alignment, generating localized interference patterns that enhance cortical entrainment through somatosensory stimulation.

Collectively, FIG. 8 illustrates modular configurations of the vibroacoustic neuromodulation platform optimized for localized, portable, and user-specific applications. The figure demonstrates how modular topper segments, transducer housings, and wireless controllers can be rearranged to suit individual therapeutic goals, from full-body relaxation to region-specific neuromodulation. The configurations maintain compatibility with the adaptive feedback and synchronization systems previously described, ensuring precise phase and frequency control across all embodiments.

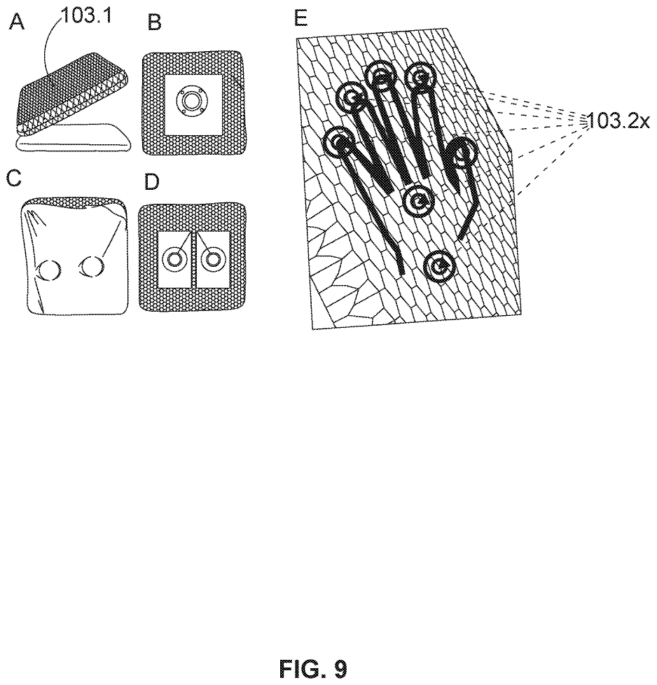

FIG. 9 illustrates multiple embodiments of the vibroacoustic transducer module assemblies (103.x) configured for use within the neuromodulation systems described herein. The figure demonstrates the structural and functional versatility of the modular transducer cushions, which may be reoriented, embedded, or arranged in multi-point arrays to conform to various anatomical regions.
View A—Flexible Transducer Cushion Assembly. View A shows a flexible cushion housing (103.1) adapted to receive a single vibroacoustic transducer element. The cushion enclosure may comprise multi-layer fabric, mesh, or elastomeric material designed to maintain body contact while allowing acoustic energy transmission. The cushion may be folded or contoured without compromising transducer alignment, permitting use on curved or irregular surfaces such as limbs or cervical regions. In certain embodiments, the cushion housing (103.1) includes an internal pocket or zipper closure for transducer insertion, as described in earlier figures, allowing the user to interchange or upgrade transducer units without replacing the entire cushion assembly.
View B—Embedded Transducer Module. View B illustrates a single transducer element positioned within a recessed cavity of the cushion housing. The cavity may include a polymeric or foam baffle to control vibration damping and directional coupling. The transducer may be sealed using a removable fabric overlay or acoustically transparent membrane. This configuration provides localized stimulation while maintaining comfort and minimizing surface vibration transmission to adjacent areas.
View C—Dual-Transducer Assembly. View C presents a dual-transducer cushion embodiment, wherein two independent vibroacoustic elements are mounted side-by-side within a shared housing. Each transducer may be individually addressable by the control unit, enabling bilateral or counter-phase drive for the generation of complex interference patterns or amplitude gradients.

The cushion surface may include preformed indentations or flexible diaphragms aligned with each transducer to optimize vibrational transfer efficiency at the user interface.
View D—Alternative Transducer Configurations. View D depicts a top-down perspective of alternate dual-transducer assemblies with varying geometries. Transducers (103.21, 103.22) are shown mounted in distinct positions within the modular pad, optionally connected via flexible leads or internal bus channels. These configurations allow frequency-specific targeting of adjacent anatomical zones, such as thoracic or lumbar segments, or can be used in synchronized patterns for harmonic propagation along the body.
View E—Distributed Multi-Point Actuator Array. View E illustrates a multi-point transducer array (103.2x) integrated into a flexible surface resembling a glove, mat, or conformal sheet. The array includes multiple miniature vibroacoustic elements arranged in anatomically or functionally relevant positions here exemplified as a hand-shaped layout with individual actuators corresponding to fingers and palm regions.

Each transducer (103.2x) is connected to a flexible conductive pathway, allowing independent control of amplitude and phase per node. This configuration enables precise spatial and temporal mapping of vibrational energy, suitable for neurorehabilitation, sensory training, or localized neuromodulation protocols targeting fine motor circuits.

The underlying substrate may include a honeycomb or hexagonal structural pattern for flexibility and load distribution, providing a lightweight, breathable platform for extended wear or portable use.

Collectively, FIG. 9 illustrates the design and functional adaptability of the modular vibroacoustic transducer assemblies used within the disclosed neuromodulation systems. The figure demonstrates how individual transducer modules (103.x) may be embedded in cushions, arranged in pairs, or distributed in multi-point arrays to deliver controlled vibrotactile stimulation across a wide range of body geometries and therapeutic applications.

These embodiments highlight the modular, interchangeable nature of the design, supporting user-specific customization, scalable manufacturing, and integration with the system's closed-loop control and adaptive feedback architecture.

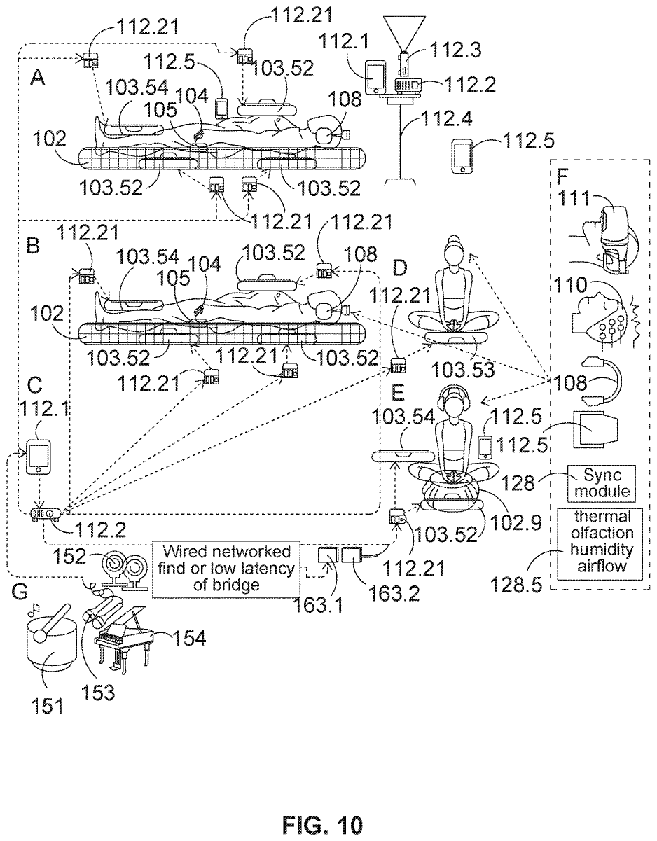

FIG. 10—Group Therapy and Network Synchronization Architecture. FIG. 10 illustrates embodiments of a networked neuromodulation and synchronization system (800) configured to coordinate multiple vibroacoustic therapy stations operating concurrently within a shared timing domain. The system enables multi-user or distributed group therapy sessions, wherein each participant's device maintains real-time synchronization of vibroacoustic, auditory, and visual outputs based on a common control signal and environmental feedback.
Views A and B—Multi-User Synchronization Overview. Views A and B depict two individual therapy stations (102) each comprising a support platform (101), topper assembly (102), transducer elements (103.52, 103.54), and user interfaces (107, 108). Each station incorporates a sensor array (104) for physiological monitoring and communicates with a wireless control network (112.1-112.5) linking all systems to a central synchronization hub (125).

The control units (110) embedded in or associated with each therapy station regulate local stimulation parameters, while synchronization modules (112.21, 112.22) receive timing data from the central sync server. These data streams maintain precise phase alignment among all active channels, ensuring that vibroacoustic, auditory, and photonic stimuli across users remain temporally coherent.

The network may be wired or wireless (e.g., Wi-Fi, Bluetooth, low-latency RF link), and may include redundancy channels (152) to maintain coordination even if one or more nodes lose direct contact with the master clock.
View C—Wireless Network and Environmental Sensor Integration. View C highlights the wireless network control architecture, including portable user interfaces (112.1, 112.2) that communicate with the central synchronization hub (125) through intermediate routers or low-latency bridges (152).

Environmental sensors (163.1, 163.2) measure parameters such as room temperature, humidity, and ambient sound to inform adaptive compensation of transducer output and timing. A network interface bridge (152) coordinates data exchange among nodes, while the synchronization logic embedded in each controller applies phase and amplitude corrections to maintain consistency across participants.

This architecture supports both co-located sessions (multiple users in one room) and distributed sessions (users in different locations connected via network).
Views D and E—Individual and Group Therapy Configurations. Views D and E show alternative use cases of the group synchronization system, including both meditative seated configurations (103.53, 103.54) and reclined therapy platforms (102.9, 102.5).

In these configurations, each participant's vibroacoustic transducer array (103.52-103.54) operates under real-time synchronization governed by the shared timing signal (112.21). The central controller (125) transmits periodic timing pulses and waveform templates that each node adapts to its user-specific entrainment model.

This arrangement allows the group to experience synchronized rhythmic patterns, binaural or multi-sensory entrainment sequences, or shared waveforms across multiple platforms—creating coherent group neuromodulation events or “entrainment ensembles.”

View F—Central Synchronization Module. View F provides detail of the central synchronization and control subsystem, comprising a sync module (128) that generates master timing and phase reference signals. The sync module communicates with multiple peripheral devices, including the main controller (110), audio output device (108), visual display or AR interface (111), and environmental compensator (125.1).

The synchronization server (128) may operate as a local processor, a cloud-based timing reference, or an external device providing standardized clock pulses to ensure millisecond-scale temporal accuracy across systems.

This synchronization allows vibroacoustic waveforms, audio, and light sequences to align not only within a single device but also across multiple users simultaneously, enabling networked entrainment or co-regulated therapy sessions.
View G—Environmental Feedback and Calibration Interface. View G depicts environmental feedback subsystems (151-154) that can be used to calibrate or augment group synchronization. These may include microphones, cameras, or laser-based distance sensors configured to measure acoustic interference patterns or user motion. The environmental analyzer (151) interfaces with a visualizer console (154) to display synchronization metrics or to validate group timing performance.

In some embodiments, the environmental analyzer automatically adjusts timing offsets for each user station to compensate for spatial separation, ensuring coherent phase alignment of vibroacoustic output across physical distances.

Collectively, FIG. 10 demonstrates the multi-user synchronization and group neuromodulation architecture (800), in which multiple vibroacoustic therapy platforms communicate through a shared wireless network to deliver phase-locked sensory stimulation across users. The figure further illustrates integration of environmental sensors, synchronization modules, and adaptive timing compensation, enabling distributed neuromodulation with group coherence and individualized adaptive control.

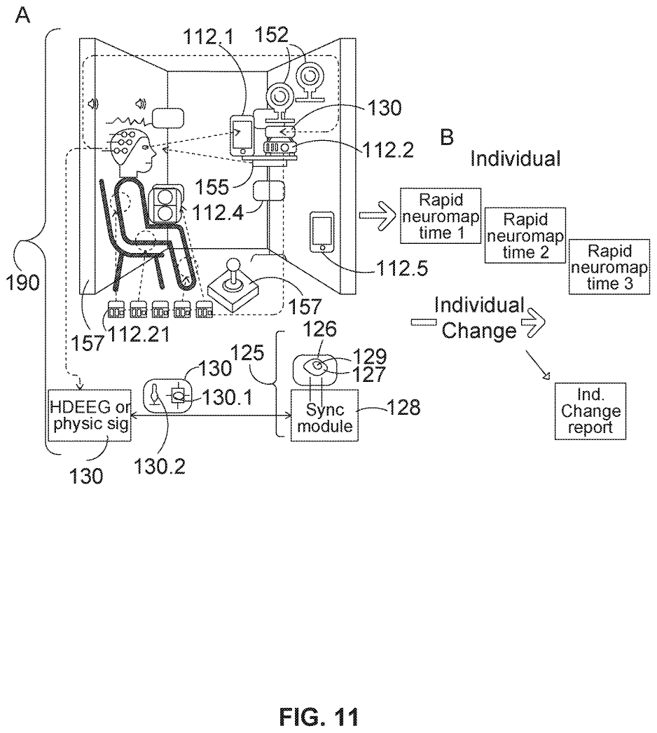

FIG. 11 illustrates an embodiment of an adaptive calibration and neuroresponse tracking system (190) configured to monitor, evaluate, and adjust vibroacoustic neuromodulation parameters in real time based on an individual's measured neural and physiological changes. The system employs closed-loop feedback incorporating high-density neural data, biometric monitoring, and synchronization logic to continuously refine stimulation models.
View A—Calibration and Feedback Environment. In view A, a user is shown positioned within a calibration environment (190) equipped with multiple sensory and recording modules. The user wears a high-density EEG headset (130) or equivalent physiological monitoring system (HDEEG or physio kit), which records neural activity across a plurality of channels in synchrony with vibroacoustic and auditory stimulation.

The user is seated on a vibroacoustic platform comprising transducer arrays (112.21) and sensor nodes integrated into the seat and footrest. These components provide tactile stimulation while simultaneously capturing vibrational and biomechanical response data. Sensors (157) positioned at contact points—such as the headrest, lumbar pad, and foot pads—transmit vibration and movement data to the local control subsystem (112.2, 112.4).

The control interface modules (112.1-112.5) manage stimulation timing, data acquisition, and synchronization with the central timing module (125) and sync unit (128). The synchronization module (128) provides master clock and phase reference data to ensure temporal alignment of sensory channels and physiological monitoring inputs.

The calibration system may include environmental monitoring components (152, 155), such as ambient light or sound sensors, that compensate for external stimuli by dynamically adjusting signal amplitude or spectral distribution. A display or visualization console (112.3) provides the operator with real-time feedback on neural signal quality, transducer phase alignment, and calibration progress.

The data processing subsystem (130.1-130.2) performs local signal conditioning and feature extraction on the recorded EEG and physiological data. These data are transmitted to an analytics server or cloud platform via network interface (125, 152) for subsequent evaluation and model updating.
View B—Individual Neuroresponse Mapping and Longitudinal Change Analysis. View B schematically illustrates how data collected through the calibration system are processed to derive individualized neuroresponse profiles over time. At successive time points (e.g., Rapid neuromap time 1, time 2, time 3), the system generates individualized neuromaps that characterize the user's cortical and physiological response to the multimodal stimulation.

An individual change analyzer computes the differential patterns between successive neuromaps to quantify adaptation, plasticity, or entrainment effects induced by the therapy. The resulting individual change report may include updated stimulation frequency bands, amplitude thresholds, or sensory weighting parameters for subsequent sessions.

Through this adaptive feedback process, the system iteratively converges toward a personalized neuromodulation profile, continuously refining stimulation timing, waveform morphology, and sensory synchronization to align with the individual's evolving neurophysiological state.

Collectively, FIG. 11 depicts the adaptive calibration and neuroresponse tracking framework (190), integrating neural and physiological sensing, synchronization logic, and analytic feedback to maintain optimal stimulation alignment over time. The illustrated system supports both local and cloud-based processing, enabling real-time calibration, longitudinal tracking, and individualized adjustment of multimodal neuromodulation protocols.

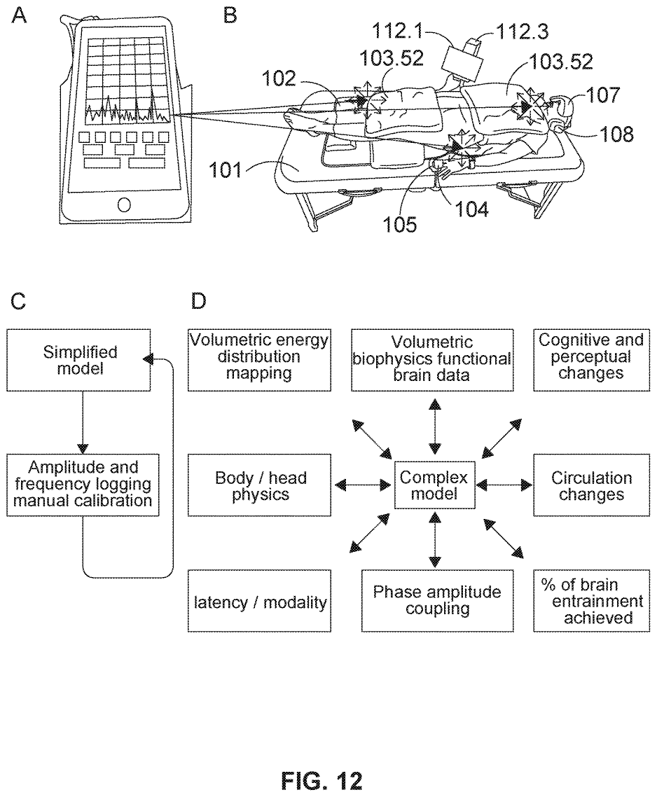

FIG. 12 illustrates embodiments of a model-based neuromodulation control and analytics system (1200) configured to model, evaluate, and optimize vibroacoustic neuromodulation parameters based on physical, physiological, and neurofunctional data. The system employs both simplified and complex adaptive models to correlate sensory stimulus parameters with neural and perceptual outcomes, facilitating closed-loop refinement of therapy protocols.
View A—Control and Calibration Interface. View A depicts a representative control and calibration interface (112.1) that allows a practitioner or automated system to monitor and adjust operational parameters of the vibroacoustic neuromodulation platform. The interface may be implemented as a tablet, dedicated controller, or software dashboard.

Displayed parameters may include individual transducer frequencies, waveform amplitudes, phase relationships, and output balance across multiple channels. During manual calibration, the operator may log amplitude and frequency values, perform latency adjustments, or initiate synchronization routines in coordination with the onboard sensors (104) and controller (110).
View B—Measurement and Feedback Configuration. View B illustrates a user positioned on the vibroacoustic therapy platform comprising the base support (101), topper assembly (102), distributed transducer elements (103.52), and physiological sensor array (104). Auditory and visual synchronization is provided through the head-mounted interface (107, 108), while the control system communicates with the calibration device (112.1, 112.3).

The transducers (103.52) deliver modulated vibroacoustic energy to the user's body, and the sensor module (104) captures resulting vibrational, physiological, and neural response data. These signals are analyzed to calibrate the amplitude, frequency, and phase of subsequent stimulations. The configuration supports iterative refinement of vibroacoustic delivery based on user-specific response metrics derived from earlier sessions.
View C—Simplified Model Loop. View C represents a simplified analytical model used during early-stage calibration or baseline operation. The simplified model employs direct amplitude and frequency logging to correlate stimulus inputs with recorded biometric outputs, allowing manual adjustment of vibroacoustic and auditory parameters.

In this mode, a human operator or basic algorithmic controller tracks user response and modifies parameters empirically. While less computationally intensive, the simplified model provides valuable initialization data for more complex modeling described in view D.
View D—Complex Multi-Parameter Modeling Framework. View D illustrates the complex model architecture, integrating multidimensional datasets derived from both system sensors and external measurement instruments. The complex model incorporates several interdependent analytical domains, including:
Volumetric Energy Distribution Mapping—quantifies mechanical energy propagation through the user's body and surface interface, based on transducer output and tissue coupling characteristics.
Volumetric Biophysics and Functional Brain Data—integrates EEG, fNIRS, or other neural imaging data with mechanical propagation data to form a unified model of sensory input versus neural activation.
Cognitive and Perceptual Changes—captures user-reported or algorithmically detected variations in alertness, relaxation, focus, or mood associated with the neuromodulatory stimulation.
Circulation Changes—models vascular and hemodynamic responses induced by rhythmic vibroacoustic stimuli, providing a physiological feedback dimension to the control loop.
Phase-Amplitude Coupling—quantifies the relationship between the vibroacoustic drive waveform and concurrent neural oscillatory patterns, establishing phase coherence metrics used for entrainment tuning.
Latency/Modality Interaction—models timing differentials between auditory, visual, and vibroacoustic pathways to ensure optimal cross-modal alignment.
Body/Head Physics—characterizes biomechanical transmission paths and resonant properties across user-specific anatomical structures.
Percentage of Brain Entrainment Achieved—provides a computed performance index indicating how closely the user's neural oscillations align with the target stimulation frequencies.

Each of these sub-models exchanges parameters with the central complex model, forming a dynamic feedback ecosystem that refines therapeutic accuracy and responsiveness over successive sessions.

Collectively, FIG. 12 demonstrates the model-based analytics and simulation layer of the vibroacoustic neuromodulation system. The integration of simplified manual calibration with advanced biophysical and neurofunctional modeling enables both immediate operational control and long-term adaptive optimization. The architecture supports individualized therapy refinement, predictive modeling of neuromodulatory effects, and quantitative evaluation of entrainment efficacy across multiple sensory modalities.

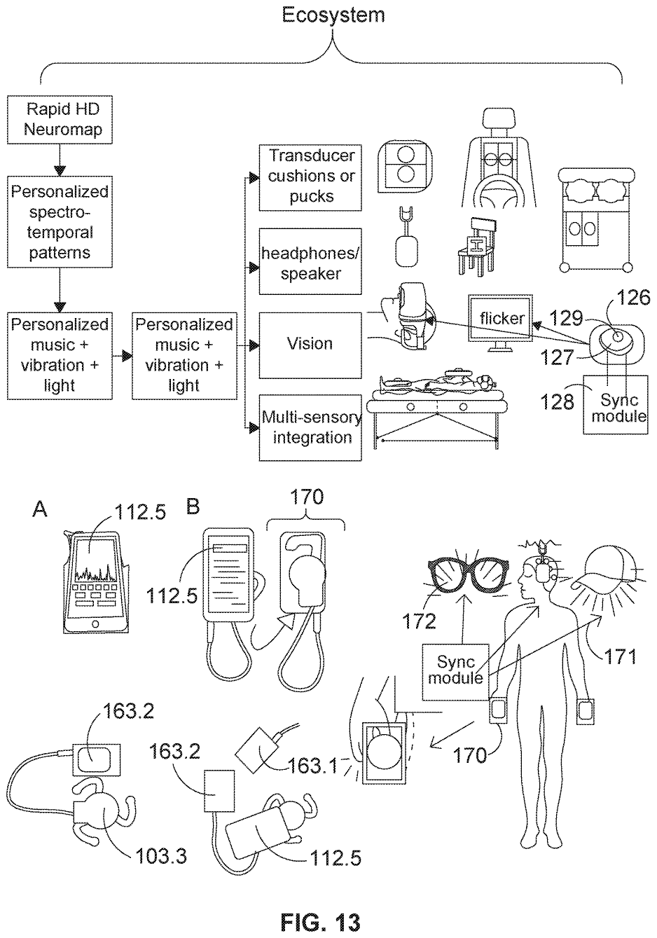

FIG. 13 illustrates an embodiment of a distributed neuromodulation ecosystem (1100) that integrates multiple hardware, software, and environmental components to form a unified adaptive sensory network. The ecosystem coordinates personalized vibroacoustic, auditory, and visual stimulation through synchronized control of local and remote modules, providing consistent neuromodulatory effects across varied use environments.

### System Overview

The ecosystem begins with acquisition of rapid high-density neuromapping data (Rapid HD Neuromap) which serve as the foundation for individualized entrainment profiles. The neuromapping process generates personalized spectro-temporal patterns, corresponding to the user's unique neural oscillatory characteristics and response signatures.

These data feed into a personalized stimulation engine that synthesizes multi-modal outputs-combining music, vibration, and light to create user-specific sensory protocols. The resulting stimuli are distributed across different hardware nodes, including transducer cushions or pucks, headphones or speakers, and visual interfaces, ensuring that vibroacoustic, auditory, and optical stimuli are delivered in precise phase and amplitude alignment.

### Ecosystem Components

Transducer Cushions and Pucks. Vibroacoustic transducer assemblies (103.x family) serve as the tactile interface for the system, translating digital stimulation signals into mechanical vibrations. These components may be integrated into mats, chairs, vehicle seats, or wearable garments, depending on the application.

Auditory and Visual Modules. Headphones or speakers deliver synchronized sound fields, while visual elements (e.g., LED arrays or flicker modules) provide photic stimulation. These elements interface with the synchronization subsystem (126, 127, 128), ensuring that phase and latency offsets are minimized across modalities.

Multi-Sensory Integration Platform. The multi-sensory integration module orchestrates timing and amplitude across all active channels, harmonizing outputs from the transducer, audio, and visual devices. In one embodiment, this module resides in the main control platform (see FIG. 10); in another, it operates via a distributed controller or cloud-based synchronization server.

Sync Module. The sync module (128) provides a master timing signal for the ecosystem, coordinating both in-room and external devices through wired or wireless communication. Environmental factors such as flicker lighting, room temperature, and sound propagation are adjusted in real time via an adaptive feedback network (125-129), maintaining consistent neuromodulatory fidelity regardless of context.

### Localized Device Examples (Views A-D)

View A—Control Tablet or Local Interface. View A shows a portable control tablet (112.5) or handheld interface used to initiate and monitor neuromodulation sessions. This device can display amplitude, frequency, and synchronization status for each sensory channel.
View B—Wearable Synchronization Node. View B illustrates a wearable synchronization device (170) which may be attached to the user's body, chair, or clothing. The wearable sync node communicates with peripheral sensors and transducers to align timing and waveform data between mobile and stationary systems.
View C—Compact Vibroacoustic Assembly. View C depicts a compact vibroacoustic or pneumatic actuator (163.1, 163.2) integrated with a small transducer cushion (103.3). This configuration provides localized stimulation—e.g., to the neck, wrist, or forearm—and can be wirelessly synchronized with the main system or operate as a stand-alone portable unit.
View D—Hybrid Wearable Interface. View D shows a hybrid wearable configuration, combining a vibroacoustic actuator (103.3) and local control electronics (112.5). This embodiment may be used for mobile entrainment or rehabilitation, functioning autonomously or under remote synchronization control via Bluetooth, Wi-Fi, or low-latency RF link.
View E—Full Ecosystem Synchronization. View E illustrates a human subject equipped with multiple synchronized sensory devices, including:
Head-mounted optical interface (171) or flicker glasses providing photic entrainment,
Wearable synchronization module (170) coordinating with the central sync system (128),
Transducer-integrated seat or mat providing vibroacoustic stimulation,
Environmental lighting interface (172) for ambient entrainment.

The sync module ensures that all subsystems operate under a unified temporal framework, thereby maintaining multimodal coherence between tactile, auditory, and visual channels. This configuration supports personal or clinical use cases, including remote neuromodulation therapy, wellness programs, and multi-user synchronized experiences.

Collectively, FIG. 13 depicts the integrated neuromodulation ecosystem (1100), encompassing data acquisition, personalized model generation, multi-sensory output coordination, and environmental synchronization. The ecosystem architecture extends the vibroacoustic neuromodulation platform into an interconnected network of synchronized devices-ranging from stationary therapeutic systems to portable wearables-creating a seamless framework for continuous, adaptive, and personalized neural entrainment across environments.

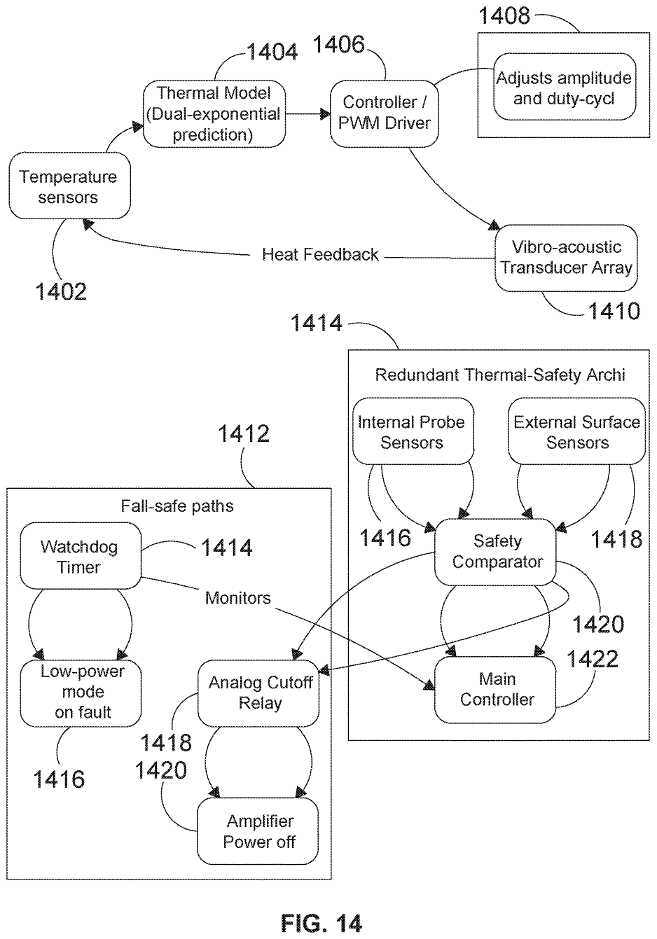

FIG. 14 illustrates embodiments of a thermal-safety and closed-loop control architecture (1300) configured to predict, monitor, and regulate the thermal behavior of vibroacoustic transducer arrays during operation. The system utilizes real-time temperature feedback and predictive modeling to ensure user safety, prevent overheating, and preserve acoustic coupling efficiency under variable drive conditions.
View 1402-1410: Predictive Thermal Feedback Control. In the upper block diagram, temperature sensors (1402) positioned at both the internal and external interfaces of the vibroacoustic transducer array provide continuous thermal measurements to a thermal model subsystem (1404). This subsystem executes a dual-exponential predictive model that estimates surface and internal temperatures in real time based on measured data, input power, and time-dependent heat dynamics.

The thermal model is coupled to a controller/PWM driver (1406), which dynamically adjusts the amplitude and duty cycle of the vibroacoustic drive signals according to the predicted temperature. This closed-loop control path regulates the transducer drive such that surface temperature remains below predefined thresholds while maintaining sufficient vibrational output for therapeutic efficacy.

The controller (1406) communicates bidirectionally with the vibroacoustic transducer array (1410). Heat feedback from the transducers is continuously sampled and relayed back to the model (1404), closing the loop to ensure stability even under sustained operation.

Experimental validation through 20-minute SAB-1060 transducer tests confirmed that the control algorithm maintains safe surface temperatures while preserving coupling efficiency, even at drive fractions up to 0.8 (corresponding to Stage-4 operational load).

### Thermal Model Parameters and Equations

The predictive model employed in block 1404 is derived from empirical testing and applies distinct formulations for heating and cooling phases.
Heating Phase (0-5 minutes):

$${T}_{s} ( t ) = {T}_{e n v} + ( {k}_{1} \cdot {P}^{0.92} ) \cdot ( 1 - {e}^{- t / {τ}_{h}} )$$

Cooling Phase (t>5 minutes):

$${T}_{s} ( t ) = {T}_{e n v} + ( {T}_{p e a k} - {T}_{e n v} ) \cdot [ α \cdot {e}^{- ( t - 5 ) / {τ}_{c 1}} + ( 1 - α ) \cdot {e}^{- ( t - 5 ) / {τ}_{c 2}} ]$$

Where:

| Parameter | Description | Typical Value | |
|---|---|---|---|
| Tₑₙᵥ | Ambient temperature | 77° | F. |
| P | Drive fraction (S1-S4) | 0.2-0.8 | |
| k₁ | Gain coefficient | 30-78° | F. |
| τₕ | Heating time constant | 1.9-2.3 | min |
| α | Fast-cooling fraction | 0.55 ± 0.05 | |
| τ_{c1} | Fast cooling constant | 4.8 | min |
| τ_{c2} | Slow cooling constant | ~17 | min |

This model enables the controller to predict peak surface temperature before it occurs, applying preemptive amplitude reduction or duty-cycle modulation to avoid exceeding safety limits. The closed-loop behavior provides smooth thermal decay curves during cooldown, avoiding abrupt intensity shifts that could disrupt neuromodulatory continuity.
View 1412-1422: Redundant Safety and Fail-Safe Architecture. The lower block diagram (1412) depicts the redundant thermal-safety architecture, which operates in parallel with the predictive feedback loop to ensure system safety even in the event of sensor or control failure.

Thermal Safety Redundancy. The redundant sensor network includes both internal probe sensors (1416) and external surface sensors (1418). These are cross-checked in real time by a safety comparator (1420). Any discrepancy or out-of-bounds reading triggers a fail-safe cascade.

Watchdog and Power Control. A watchdog timer (1414) monitors controller execution and sensor polling intervals. Upon fault detection—such as stalled communication, over-temperature, or anomalous current draw—the system transitions into low-power mode (1416).

The analog cutoff relay (1418) serves as a hardware-level disconnection path that disables amplifier output independent of software control. An amplifier power-off circuit (1420) enforces total shutdown if both the analog and digital safeguards concur on a fault condition.

Main Controller Oversight. A main controller (1422) receives data from both the safety comparator (1420) and the predictive model subsystem, integrating the results to confirm system integrity. The main controller may log events, adjust predictive parameters, or trigger visual indicators for operator awareness.

Functional Integration. Together, the predictive thermal model (1404) and redundant safety subsystem (1412-1422) provide a multi-tiered protection framework. The predictive loop offers smooth, anticipatory control, while the hardware fail-safe ensures absolute thermal containment even under unexpected fault conditions. This dual-path approach satisfies medical device safety criteria for continuous-contact vibroacoustic systems.

FIG. 14 demonstrates the thermal-safety and closed-loop control subsystem (1300), comprising predictive modeling, real-time feedback, and redundant fault protection for vibroacoustic neuromodulation systems.

Validated through SAB-1060 testing, the model accurately predicts surface temperature rise and decay under varied drive loads, maintaining safe operation without compromising vibrational fidelity. The redundant analog and digital safeguards guarantee safety compliance and ensure uninterrupted, adaptive performance across prolonged therapeutic sessions.

Temperature management implements a dual-exponential predictor with a 42° C. cutoff; a watchdog and analog cutoff relay force amplifier low-power on threshold or controller fault.

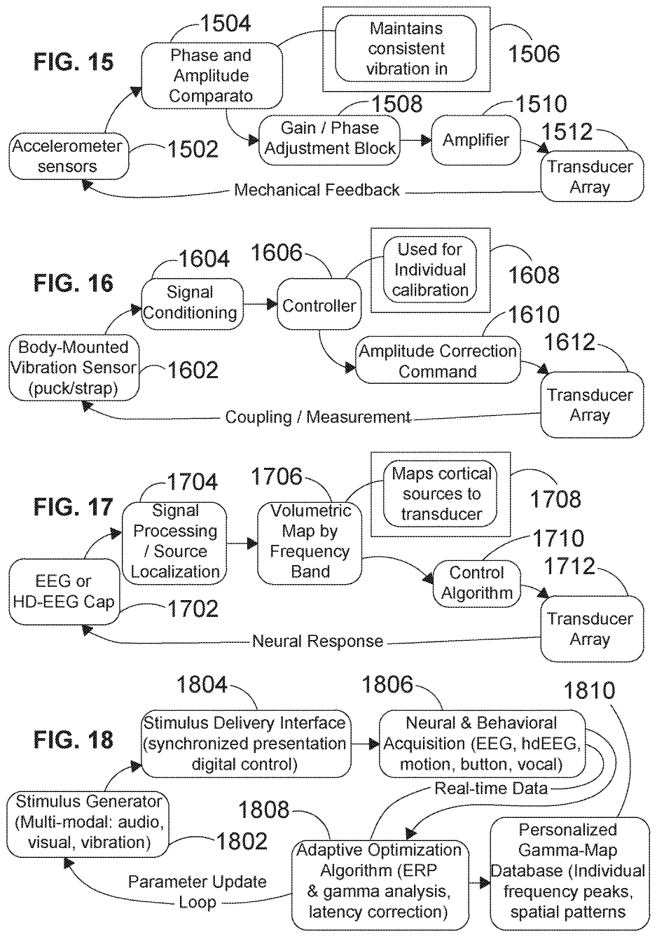

FIG. 15 illustrates an embodiment of a vibration monitoring and amplitude control subsystem (1400) that employs accelerometer feedback and phase comparison to maintain consistent mechanical vibration amplitude, waveform fidelity, and phase coherence in the vibroacoustic transducer array.

The subsystem forms part of the closed-loop control hierarchy, providing mechanical domain feedback complementary to the thermal and neural feedback loops described in FIGS. 14 and 17.

Accelerometer Feedback Path (1502-1508). A set of accelerometer sensors (1502) is mounted on or adjacent to the vibroacoustic transducer array. These sensors continuously measure the instantaneous acceleration and displacement of the transducer surface during operation.

Sensor outputs are routed to a phase and amplitude comparator (1504), which evaluates the measured signal against a reference waveform derived from the system's internal oscillator or command input. This comparator calculates the instantaneous phase difference and amplitude deviation between the desired and actual vibration signals.

The comparator (1504) outputs two control signals: A phase correction signal to realign vibration timing, and an amplitude error signal proportional to deviation from the target vibration magnitude. These signals are transmitted to a gain/phase adjustment block (1508), which dynamically modifies the amplifier's drive parameters.

Dynamic Gain and Phase Compensation (1508-1510). The gain/phase adjustment block (1508) provides the core adaptive element of the system. It applies compensatory scaling to the transducer drive waveform, increasing or decreasing gain or applying phase offsets as needed to preserve waveform fidelity.

The adjusted signal is then passed to an amplifier (1510), which delivers the corrected power waveform to the transducer array (1502).

This closed-loop signal path ensures that mechanical vibration amplitude and phase remain stable even as user load, transducer temperature, or surface impedance change during operation.

Mechanical Feedback Stability and Consistency (1506). A stability controller (1506) continuously monitors the comparator output and amplifier drive feedback to maintain consistent vibration intensity within defined tolerances. This function may include proportional-integral-derivative (PID) or adaptive control logic that smooths transient fluctuations, ensuring stable sensory perception for the user.

Through this real-time mechanical feedback loop, the system maintains constant vibrational energy delivery across transducers, minimizing perceptible drift in intensity or synchronization error.

Functional Summary: The vibration monitoring and amplitude control loop (FIG. 15) integrates accelerometer-based feedback, dynamic gain/phase correction, and real-time stability control to form a self-regulating mechanical feedback system.

It continuously compensates for environmental, physiological, and mechanical variations, thereby preserving consistent tactile stimulation. This mechanical feedback layer interfaces directly with the thermal feedback loop (FIG. 14) and neural phase control system (FIG. 17), forming one component of the broader closed-loop neuromodulation control architecture.

FIG. 16 illustrates an embodiment of a user-mounted vibration monitoring and amplitude calibration subsystem (1500) configured to measure vibroacoustic coupling between the transducer array and the user's body, and to dynamically adjust transducer output based on measured transmission efficiency.

This subsystem establishes a wearable mechanical feedback loop, complementing the fixed accelerometer loop of FIG. 15, to maintain consistent perceived vibration amplitude across individuals and sessions.

Body-Mounted Vibration Sensor (1602). A body-mounted vibration sensor (1602)—implemented as a lightweight puck, strap, or patch sensor—is positioned at one or more anatomical sites such as the sternum, lumbar region, or limbs.

The sensor contains a high-sensitivity accelerometer or piezoelectric element configured to detect the magnitude and spectral content of vibrational energy transmitted through body tissues during stimulation.

The sensor's signals represent the actual coupling efficiency between the vibroacoustic platform and the user's body, enabling the system to compensate for variations due to user position, clothing thickness, or body mass.

Signal Conditioning and Controller Interface (1604-1606). The sensor output is routed through a signal conditioning circuit (1604), which amplifies, filters, and digitizes the raw vibration data. This circuit normalizes the signal for comparison with the system's drive reference, removing noise and environmental interference.

A controller (1606) processes the conditioned signal to compute the vibration transmission ratio (VTR)—the ratio of actual body vibration amplitude to commanded transducer amplitude. This ratio quantifies how effectively vibroacoustic energy couples into the user's body at any given time.

The controller compares the measured VTR to a calibrated target range and generates an amplitude correction command (1610) when deviations occur.

Adaptive Amplitude Correction Command (1610-1612). The amplitude correction command (1610) dynamically modifies the output drive signal of the transducer array (1612). When the measured coupling efficiency falls below the optimal threshold—for instance, when the user shifts position or the mattress contour changes the controller increases drive amplitude to restore target energy delivery.

Conversely, if excessive coupling is detected, the controller proportionally reduces amplitude to maintain comfort and prevent overstimulation.

In some embodiments, correction occurs independently across multiple transducer channels, allowing spatially adaptive control of localized vibration zones (e.g., head, torso, feet). This ensures even distribution of tactile energy without perceptible hotspots or dropouts.

Individualized Calibration Mode (1608). The individual calibration process (1608) uses the same body-mounted sensor data to build a personalized coupling profile for each user. During initial setup or periodic recalibration, the controller varies output amplitude across a defined frequency sweep, measuring the corresponding response at the body-mounted sensor.

This process generates a frequency-dependent coupling map, which is stored in user-specific memory and used to compensate for frequency-dependent absorption or anatomical differences.

Over time, the calibration data enable automatic compensation, ensuring repeatable vibroacoustic experiences across sessions and across different users on the same hardware platform.

Functional Summary: The subsystem of FIG. 16 extends the closed-loop control system by incorporating direct body-mounted feedback into the amplitude regulation process. Together with the accelerometer-based mechanical feedback of FIG. 15, this wearable loop forms a dual-sensing framework that maintains both mechanical drive stability and biomechanical coupling fidelity.

This ensures that each user receives a precisely controlled and perceptually uniform vibroacoustic experience, regardless of position, load, or body composition—an essential requirement for therapeutic consistency and safety.

FIG. 17 illustrates an embodiment of an EEG- or hdEEG-integrated feedback control subsystem (1600) configured to monitor neural entrainment and dynamically adjust vibroacoustic stimulation parameters based on real-time cortical responses.

The system establishes a closed-loop neural feedback channel, enabling adaptive modulation of stimulation frequency, phase, and amplitude to align sensory inputs with the user's endogenous oscillatory rhythms.

EEG/hdEEG Signal Acquisition (1702). A neural acquisition interface (1702)—such as a standard EEG cap or high-density EEG (hdEEG) array—records brainwave activity from multiple scalp locations. The system monitors both spontaneous (resting-state) and evoked responses during vibroacoustic stimulation.

Electrode configurations may be optimized for detecting gamma, beta, alpha, or theta oscillatory components depending on the therapeutic target. In hdEEG embodiments, high spatial resolution enables mapping of localized cortical sources associated with sensory entrainment.

Signal Processing and Source Localization (1704). EEG data are processed in real time by a signal processing and source localization module (1704). This module filters and decomposes neural signals into component frequency bands using methods such as Fast Fourier Transform (FFT) or Independent Component Analysis (ICA).

Using inverse modeling algorithms, the system computes source localizations that identify active cortical regions correlated with vibroacoustic or auditory stimulation. This spatial and temporal decomposition produces a neural activation map, allowing the controller to determine which brain regions are responding to each modality.

Volumetric Frequency Mapping (1706). The volumetric map by frequency band (1706) consolidates neural response data into a three-dimensional model of cortical activation strength across frequencies.

This mapping reveals how specific transducer drive frequencies or phase relationships influence neural synchrony and coherence within different brain regions. By quantifying phase-amplitude coupling (PAC) and coherence indices, the system determines whether entrainment is occurring effectively at the targeted neural frequencies.

The volumetric map serves as a real-time diagnostic model that links stimulation drive parameters with observed neural dynamics, closing the analytical loop between device output and cortical outcome.

Adaptive Control Algorithm (1710-1712). A control algorithm (1710) continuously receives updated neural metrics from the volumetric mapping subsystem and compares them to a target entrainment profile derived from the user's baseline neuromap (e.g., FIG. 4).

When deviations occur—such as suboptimal phase-locking, drift in entrainment frequency, or reduced coherence—the algorithm dynamically adjusts one or more parameters of the transducer array (1712), including: Frequency and phase of vibration waveforms, amplitude scaling for specific transducer zones, and synchronization between vibroacoustic, auditory, and visual stimuli.

These updates may occur on sub-second timescales, enabling fine-grained alignment of external stimuli with the user's neural oscillatory state.

Neural Entrainment Feedback Loop (1708). The neural feedback loop (1708) represents the closed control path between measured cortical response and stimulation output. The system continuously refines vibroacoustic drive parameters to maximize neural phase-locking and sustain entrainment coherence over time.

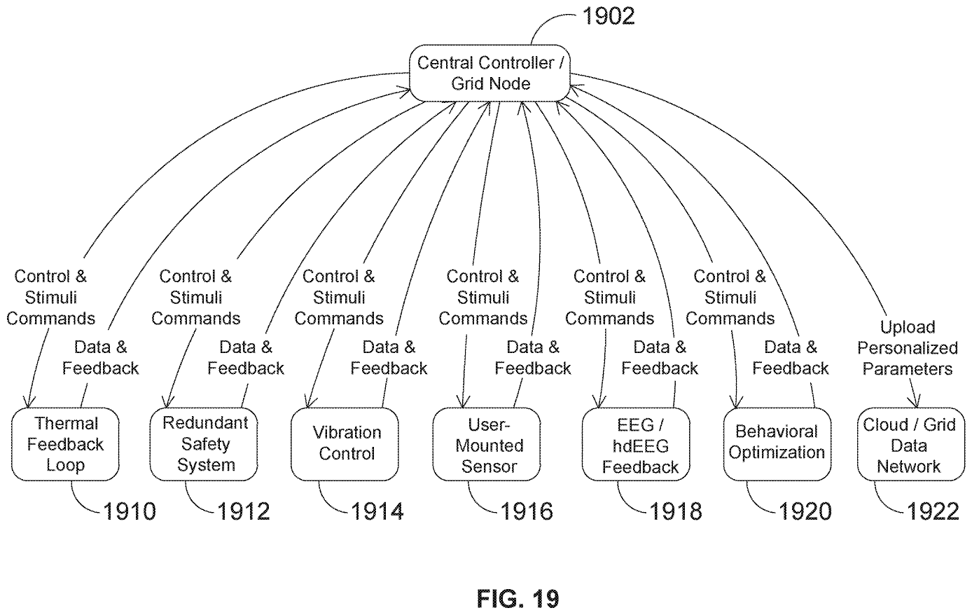

This loop integrates seamlessly with the amplitude and mechanical loops (FIGS. 15-16) and the behavioral and psychophysical feedback loops (FIGS. 18-19), forming a unified multi-modal adaptive architecture.

Functional Summary: FIG. 17 depicts the neurophysiological feedback control subsystem a cornerstone of the adaptive neuromodulation architecture. By integrating EEG or hdEEG data into the feedback network, the system achieves direct, individualized neural tuning in real time.

This configuration transforms the vibroacoustic system from an open-loop stimulus generator into a self-adaptive neural entrainment platform, capable of optimizing therapeutic output based on the user's live brain activity.

FIG. 18 illustrates an embodiment of a behavioral psychophysics and gamma optimization subsystem (1700) configured to perform rapid frequency mapping and threshold adjustment based on combined neural, behavioral, and perceptual feedback.

The system continuously analyzes how users' cognitive and perceptual states evolve in response to multi-sensory stimulation, iteratively adjusting frequency, amplitude, and phase to maximize gamma-band entrainment and perceptual comfort.

Stimulus Generator (1802). A stimulus generator module (1802) produces synchronized multimodal stimuli—including auditory, visual, and vibroacoustic waveforms—across multiple output channels.

Each stimulus type is derived from a common timing reference, ensuring phase coherence across modalities. The generator is capable of varying frequency, duty cycle, and intensity according to real-time control inputs.

This module serves as the initiating node in the behavioral feedback loop, providing the controlled sensory environment for psychophysical testing and adaptive entrainment.

Stimulus Delivery and Parameter Logging (1804). A stimulus delivery interface (1804) coordinates the precise timing and presentation of the generated waveforms. It may operate under manual, semi-automated, or fully algorithmic control.

This module logs all delivered parameters frequency, amplitude, latency, and waveform type in a parameter update loop, allowing for correlation between specific stimulus conditions and recorded neural or behavioral responses.

In some embodiments, the parameter logging engine supports rapid sweep protocols for gamma frequency identification (typically 38-52 Hz), allowing individualized mapping of neural resonance points.

Neural and Behavioral Acquisition (1806). The neural and behavioral acquisition subsystem (1806) collects both objective and subjective response data.

Objective data may include EEG-derived measures such as phase-locking value (PLV), event-related synchronization (ERS), or coherence index.

Behavioral data may include user interactions (e.g., button responses, reaction times) or performance metrics in concurrent cognitive tasks.

Together, these data streams characterize both neural engagement and behavioral entrainment, enabling the system to detect optimal resonance conditions for each individual.

Adaptive Optimization Algorithm (1808). The adaptive optimization algorithm (1808) analyzes incoming data from the acquisition subsystem to refine stimulation parameters.

This algorithm computes the relationship between frequency-dependent entrainment efficiency and subjective comfort or focus, using both real-time and historical datasets.

Machine learning or regression-based estimators may be used to model non-linear relationships between drive parameters and cortical response, automatically adjusting frequencies and amplitudes to converge on the most effective configuration.

The algorithm may also apply cross-modal latency correction, synchronizing the onset timing between visual flicker, vibroacoustic pulses, and auditory beats to minimize perceptual desynchronization.

Personalized Gamma Map Update (1810). The personalized gamma map update module (1810) integrates the optimization results into the user's individual entrainment profile.

This map specifies the user's peak gamma resonance frequency, preferred amplitude ratios across modalities, and adaptive thresholds for comfort and arousal.

In subsequent sessions, the system retrieves this map to initialize default parameters, ensuring that stimulation begins near the individual's empirically validated resonance zone.

Over repeated sessions, the gamma map continuously refines itself through incremental updates, improving precision and long-term stability of the entrainment model.

Functional Summary: FIG. 18 demonstrates the behavioral psychophysics and gamma optimization subsystem, which establishes a hybrid feedback framework combining neural entrainment metrics with subjective behavioral response.

This subsystem fine-tunes the vibroacoustic neuromodulation parameters through continuous adjustment of frequency, phase, and amplitude until the user's measured neural and behavioral responses achieve maximum alignment with the target entrainment state.

Together with the EEG feedback of FIG. 17 and the control architecture of FIG. 19, this system enables fully adaptive, neuro-personalized stimulation, guided by both physiology and perception.

FIG. 19 illustrates an embodiment of a unified multi-modal control architecture (1800) that integrates all primary feedback pathways—thermal regulation, mechanical vibration control, body coupling calibration, EEG/hdEEG neural entrainment, and behavioral psychophysics—into a centralized controller and networked synchronization grid.

The system functions as an intelligent neuromodulation ecosystem capable of self-calibration, adaptive entrainment, and predictive safety management in real time.

Central Controller and Synchronization Grid (1902). At the core of the architecture is a central controller and synchronization grid (1902), which coordinates timing, amplitude, and phase across all sensory output and feedback channels.

The grid maintains global temporal alignment between vibroacoustic, auditory, and visual stimuli, ensuring phase coherence to within sub-millisecond precision. It also synchronizes feedback inputs from multiple subsystems, harmonizing data streams of differing latency and sampling rate.

The central controller executes multi-threaded signal processing, safety monitoring, and adaptive optimization routines, distributing control signals to peripheral nodes responsible for thermal control, vibration drive, and neural modulation.

Integrated Feedback Loops. Each feedback loop operates as a semi-autonomous subsystem with its own local control path but is integrated into the global timing and control domain through the central controller (1902).

Thermal Feedback Loop (1910). The thermal feedback loop (1910) derives from the predictive modeling system of FIG. 14. It continuously monitors transducer temperature via internal and external sensors and adjusts drive amplitude and duty cycle through predictive heat modeling.

Thermal data are streamed into the grid for correlation with power, impedance, and environmental parameters, ensuring optimal energy transfer without thermal risk.

Mechanical Feedback Loop (1912). The mechanical feedback loop (1912) originates from the accelerometer-based vibration monitoring system of FIG. 15. It regulates vibration amplitude and phase through real-time accelerometer data, maintaining waveform integrity and consistency under varying mechanical load conditions.

Mechanical metrics such as amplitude stability, resonance alignment, and surface acceleration are continuously logged and transmitted to the central controller for dynamic gain compensation.

Body-Coupling Feedback Loop (1914). The user-mounted coupling feedback loop (1914) integrates signals from wearable vibration sensors (FIG. 16), measuring body-transmitted vibrational amplitude to ensure effective coupling.

This loop enables individual calibration and real-time adjustment of transducer power to maintain uniform perceptual intensity across anatomical regions.

Neural Feedback Loop (1916). The EEG/hdEEG feedback loop (1916) (FIG. 17) measures cortical entrainment strength, coherence, and phase synchronization relative to target neural frequencies (e.g., gamma band).

The loop outputs entrainment metrics to the controller, which adjusts drive frequency, waveform symmetry, and cross-modal timing to reinforce sustained neural synchronization.

Behavioral Feedback Loop (1918). The behavioral psychophysics loop (1918) (FIG. 18) incorporates user task performance, reaction times, and subjective feedback to refine cognitive and perceptual alignment with stimulation.

This loop links psychometric data with neural entrainment quality, allowing the system to identify the “sweet spot” between physiological engagement and user comfort.

Cross-Loop Coordination and Adaptation (1920-1922). The multi-loop adaptation manager (1920) acts as a supervisory layer that fuses all incoming data into a unified state-space representation of system and user status.

Through data fusion, the controller identifies cross-domain relationships—for example: Correlating thermal load with vibration amplitude, linking body-coupling efficiency to neural entrainment strength, adjusting stimulus phase alignment to compensate for latency shifts induced by hardware temperature or neural drift.

A global optimization engine (1922) continuously adjusts all system variables—frequency, amplitude, phase, waveform type, and modulation depth—to maximize the overall entrainment index, defined as a weighted function of neural coherence, perceptual comfort, and safety margins.

The unified control architecture shown in FIG. 19 represents the final integration of all preceding subsystems into a coherent adaptive control environment.

Through its central synchronization grid and layered feedback hierarchy, the system continuously: Predicts and regulates thermal conditions (FIG. 14), Stabilizes mechanical vibration output (FIG. 15), Optimizes body coupling (FIG. 16), Measures and reinforces neural entrainment (FIG. 17), and Refines stimulation parameters through behavioral and cognitive data (FIG. 18).

Together, these interacting loops form a multi-modal, self-learning neuromodulation network capable of maintaining stable, safe, and personalized stimulation across time and user conditions.

The architecture exemplifies the system's ability to converge all feedback domains—physical, physiological, and psychological—into a unified, intelligent therapeutic control framework.

### General System Configuration

In accordance with various embodiments, the disclosed system comprises a modular, reconfigurable vibroacoustic neuromodulation platform configured to deliver individualized therapeutic stimulation through coordinated, multi-modal sensory output. The system is operative to integrate vibroacoustic, auditory, visual, and optional olfactory or thermal stimuli under precise temporal and spectral control.

The system incorporates one or more high-density electroencephalography (HD EEG) subsystems for capturing neural activity in real time, enabling adaptive feedback loops based on user-specific brainwave characteristics. Latency mapping and phase synchronization modules are employed to ensure accurate alignment of sensory modalities, facilitating entrainment or desynchronization of neural rhythms as required for therapeutic effect.

In certain embodiments, the system further supports sensory deprivation states (e.g., visual occlusion, auditory dampening) in combination with controlled sensory delivery to amplify neuromodulatory impact. System parameters may be configured manually, algorithmically, or through hybrid interfaces that combine artificial intelligence with operator oversight.

The platform is operable in both individual and multi-user modes, supporting synchronous or differentiated stimulation profiles across users. Additionally, the architecture permits deployment in stationary, portable, or wearable configurations, enhancing accessibility and functional versatility across clinical, home, mobile, or wellness environments.

Core Components. The system comprises a plurality of structural and operational components configured to cooperatively deliver personalized, multi-modal neuromodulation through vibroacoustic, auditory, visual, and biometric interfaces. These components include, without limitation, a base platform, modular transducer assemblies, control electronics, sensor and biometric monitoring units, and user interface modules. Each of these subsystems is described in greater detail below.

Base Platform. The system includes a base platform, which may be implemented as a foldable, rollable, or fixed-body mat or therapeutic table. In certain embodiments, the platform comprises three or more interlinked segments formed from high-density foam, thermoplastic honeycomb, or other resilient structural materials. The base platform is configured to support the user during therapy and to house one or more embedded signal pathways, vibration channels, or sensor arrays. The base may further comprise integrated or attachable perimeter structures that form a raised boundary region configured to deliver lateral vibrational input to the user's flanks, limbs, or torso.

Modular Transducer Assemblies. The system comprises a plurality of vibroacoustic transducer elements, which may be inserted into receptacles formed within the base platform, topper modules, or wearable accessories. These transducers may include electromagnetic, piezoelectric, or other vibratory actuators capable of generating frequency-specific stimuli. In various embodiments, transducers may be positioned beneath, adjacent to, or above the user, enabling generation of a three-dimensional vibrational field encompassing the user's body. Transducers may be powered via wired signal lines routed through the platform or connected wirelessly, such as via Bluetooth or other low-latency protocols. Certain embodiments may include internal power sources, such as rechargeable batteries, disposed within the transducer modules.

Topper and Overlay Modules. Disposed atop the base platform is a modular topper layer, which may comprise foam, gel, air cell, or composite materials. The topper is configured to enhance comfort, improve vibrational coupling, and facilitate ergonomic adaptability for different use cases (e.g., supine therapy, seated meditation, rehabilitation protocols). The topper may further include embedded sensor arrays, surface-mount transducers, or visual stimulation modules.

Control Electronics. The system includes a central control unit configured to orchestrate signal generation, timing, distribution, and feedback processing across all operational modules. The control unit may include one or more processors, signal amplifiers, synchronization modules, and memory elements. Functions performed by the control unit include: Adjustment of transducer amplitude, frequency, and phase. Generation of synchronized multisensory stimuli across auditory, visual, and vibrotactile channels. Implementation of real-time feedback loops based on biometric sensor data. Execution of latency correction and phase alignment algorithms.

Synchronization across modalities and users may be maintained via one or more timing modules (e.g., modules 126, 127, 128), which may include redundant photonic, acoustic, or vibratory markers to ensure phase coherence and drift correction.

Sensor and Biometric Monitoring Units. The system is operably coupled to one or more sensor arrays configured to acquire physiological and environmental data during use. Such sensors may include: High-Density Electroencephalography (HDEEG) arrays (e.g., 280-channel cap) for real-time acquisition of brain activity data. Accelerometers configured to measure vibrational amplitude, frequency, and phase at discrete body locations or within transducer modules. Microphones adapted to capture environmental audio, vocal perturbations, or mechanical feedback from transducer operation. Photodiodes or photocells for detecting light emissions and synchronizing visual stimuli. Physiological sensors configured to measure parameters including, but not limited to, heart rate, galvanic skin response, respiration, and temperature. Sensor data may be utilized in closed-loop control frameworks to modulate stimulation parameters dynamically, based on real-time user response or preconfigured thresholds.

User Interface Module. The system includes one or more graphical user interface (GUI) devices (e.g., tablet, touchscreen controller) configured to allow an operator or user to initiate sessions, monitor system performance, and view biometric data. The interface module may further include functions for session customization, impedance diagnostics, user authentication, and post-session reporting. In certain embodiments, AI-assisted configuration engines may guide the selection of therapeutic modes or goal-based session profiles, based on interview data or historical session metrics.

Optional Accessories and Enhancements. In some embodiments, the system may further comprise auxiliary modules including, but not limited to: Light shields or hoods for visual isolation. Augmented or virtual reality (AR/VR) headsets for immersive content delivery. Portable calibration tools with integrated spectrogram displays. Multi-user synchronization systems for networked group sessions.

Each of the foregoing components may be modular, reconfigurable, and interoperable with other disclosed systems to form a comprehensive sensory neuromodulation platform.

### System Integration and Control Interfaces

In accordance with various embodiments, the system comprises one or more control interfaces configured to manage operational parameters and therapeutic delivery across the integrated multi-modal stimulation platform. The control interfaces may include, but are not limited to, graphical user interfaces (GUIs) rendered on smart devices, tablets, computer terminals, or dedicated hardware controllers.

Such interfaces permit the configuration of session-specific parameters including, but not limited to: transducer amplitude, frequency range, timing, waveform characteristics, and inter-modality synchronization across vibroacoustic, auditory, and visual outputs. In certain embodiments, the interface enables real-time monitoring and adjustment of system outputs in response to biometric or neurological feedback.

Additionally, the control interfaces support the creation, storage, and recall of user-specific therapy profiles, allowing individualized session programming and longitudinal tracking. The system may further incorporate access management protocols and session logging for clinical, research, or regulatory compliance purposes.

### Modular Base Platform

Foldable Mat Design and Materials. In accordance with various embodiments, the system comprises a modular and foldable vibroacoustic therapy system configured to deliver customizable, body-targeted stimulation through a portable and reconfigurable architecture. The system is operable in a wide range of therapeutic environments including, but not limited to, clinical treatment centers, wellness spas, research laboratories, residential spaces, and mobile or travel-based contexts.

The base platform of the system serves as a structurally robust and ergonomically adaptable foundation designed to support a user in multiple postures, including fully supine, semi-reclined, or seated configurations. The platform comprises a plurality of interconnected panels—typically three or four—constructed from vibration-transmissive materials such as high-density foam, thermoplastic honeycomb, or multilayer composite structures incorporating acoustic damping and resilient support layers. Each panel may have a thickness between approximately 1.5 and 2 inches, and when unfolded the full mat may measure approximately 72 inches in length and 24 inches in width, providing a full-body therapy surface.

The interconnected panels are joined via flexible hinges, fold seams, or segmented coupling joints that enable the platform to be folded into compact configurations for storage or transport. In certain embodiments, magnetic or snap-fit fasteners are employed to maintain alignment between panels when deployed in the open position. The foldable construction allows the mat to be converted between configurations, including: (a) a full-length flat configuration for supine therapy; (b) a Z-shaped configuration suitable for chair-based or meditation use; and (c) an L-shaped configuration for partial support of the torso and legs.

Each panel includes one or more recessed cavities or modular cutouts designed to receive vibroacoustic transducer pucks or other actuation modules. The cutouts are dimensioned to accept standardized transducer cartridges, enabling the user or practitioner to reposition or replace transducers to target specific anatomical regions—such as the spine, lumbar area, hips, or extremities—according to individualized therapeutic protocols. The modular receptacles may include compliant foam housings or elastomeric sleeves to maintain optimal coupling and minimize vibration loss.

Signal and power transmission between panels is accomplished via a signal infrastructure embedded within or between the layers of the mat. In certain embodiments, flexible wiring harnesses are laminated within the foam structure, while in others, conductive textile layers provide analog or digital transmission paths. Electrical continuity across fold joints may be maintained using magnetic couplers, flexible ribbon conductors, or quick-disconnect connectors. In alternative embodiments, the system may operate wirelessly, wherein each transducer puck includes an internal power source, such as a rechargeable battery, and a wireless transceiver employing Bluetooth®, Wi-Fi®, or proprietary low-latency RF communication protocols for synchronization and control.

The transducers are driven by a central amplifier or controller that modulates amplitude, frequency, and phase across multiple output channels. The modular electrical and mechanical architecture enables rapid assembly, disassembly, and cleaning without specialized tools.

When deployed on a treatment platform such as a massage table or reclining frame, the mat may function as a standalone vibroacoustic delivery system or as an auxiliary layer augmenting an existing therapeutic base. In either mode, the system is capable of delivering both sub-audible and audible frequency components for immersive full-body stimulation. Dynamic zoning of vibrational energy may be implemented, allowing specific transducer panels to operate at varying intensities or frequencies to focus energy on selected body regions.

In folded configurations, the mat supports seated, semi-reclined, or compact travel modes. For example, when folded into a chair-like arrangement, transducer elements can be positioned to deliver focused stimulation to the lumbar spine, hips, or lower limbs, facilitating applications such as mindfulness, stress reduction, or seated neuroentrainment exercises.

The system is compatible with auxiliary modules, including synchronized auditory components (e.g., headphones or integrated speakers), light-based stimulation devices (e.g., flicker or color-modulated LEDs), and physiological sensor systems (e.g., EEG, heart rate, or galvanic skin response sensors). These components may communicate with the mat's control electronics to form an integrated multi-modal feedback and neuromodulation environment.

The described foldable modular mat configuration provides high-resolution spatial control over vibrational energy distribution while maintaining portability and ergonomic adaptability. The design facilitates rapid deployment across diverse physical environments, simplifies maintenance and transport, and supports precise, user-specific neuromodulation and therapeutic applications. Accordingly, the foldable vibroacoustic mat constitutes a versatile and scalable foundation within the broader multi-sensory therapeutic system.

### Transducer Layout and Reconfigurability

In accordance with various embodiments, the system includes a plurality of vibroacoustic transducer elements configured for modular deployment across multiple axes of the therapy platform. These transducer elements may be positioned beneath, above, or laterally adjacent to the user to establish a three-dimensional vibrational field. This spatial configuration enables immersive multisite stimulation and enhances neuromodulatory precision through comprehensive vibrational coverage.

The transducers are modular in nature and may be removably coupled to designated receptacles or mounts located on or within the therapy platform. The configuration allows practitioners or users to attach, detach, reposition, or exchange transducer elements based on individualized treatment protocols, anatomical considerations, or desired vibrational profiles. This user-directed customization supports flexible therapeutic targeting across distinct body zones including, but not limited to, the spine, limbs, core musculature, and lateral body surfaces.

The system includes a base support structure or platform upon which the user is positioned during therapy. Above this base, a modular topper module is deployed, comprising interchangeable layers such as viscoelastic foam inserts, gel pads, or pneumatic overlays. These toppers provide ergonomic support and can be swapped to accommodate user preferences, clinical objectives, or application-specific requirements (e.g., high-precision research use versus comfort-oriented relaxation therapy).

Transducer elements may be embedded within, affixed to, or positioned in proximity to both the base platform and the topper module. This dual-axis architecture permits bidirectional stimulation—delivering vibrational input both upward from beneath the user and downward from above—thereby forming a vibrational cocoon or envelope surrounding the user's body. This configuration facilitates: Immersive, whole-body vibrational stimulation, enhancing the perception and physiological efficacy of entrainment signals; Targeted energy modulation to specific anatomical regions, enabling selective intensification or attenuation in localized zones; Multichannel coordination, whereby separate transducers may operate asynchronously or in synchrony to generate phase-differentiated waveforms or frequency-specific entrainment profiles.

In some embodiments, localized transducer pucks are employed to deliver high-intensity vibrational stimulation to specific body parts, such as the feet, hands, or cervical spine. These pucks may be manually positioned or magnetically coupled to designated locations and are electrically connected to the central control module either via embedded wiring or wireless transmission.

The system further includes integrated physiological sensors operable to monitor biometric indicators such as heart rate, galvanic skin response, respiratory patterns, or thermal flux. Data from these sensors is used in real time to modulate stimulation parameters—such as amplitude, frequency, and waveform shape—thereby enabling closed-loop feedback control of the therapy session based on dynamic user states.

An optional light shield may be affixed to the upper structure to reduce ambient light exposure, facilitating sensory isolation. Additionally, synchronized auditory stimulation may be delivered via headphones or speaker modules integrated into the platform or auxiliary accessories. Auditory signals are temporally aligned with vibrational stimuli to enhance multisensory coherence and improve neuromodulatory outcomes.

The system incorporates a control interface, accessible via touchscreen, computing device, or dedicated control panel. This interface allows the user or practitioner to configure and monitor therapy parameters including, but not limited to, transducer frequency, amplitude, phase offset, duration, and stimulation profiles. A real-time visualization display may also be provided to indicate the operational status of each transducer, the physiological state of the user, and the synchronization of multimodal sensory elements.

Collectively, the modular transducer architecture and reconfigurable layout enable adaptive, precision-targeted vibroacoustic therapy across a wide range of user postures, clinical indications, and operational environments.

### Topper Modules and Ergonomic Adaptability

In various embodiments, the system comprises a plurality of interchangeable topper modules operable to interface mechanically and acoustically with the underlying base platform. Each topper module is configured to augment the system's ergonomic characteristics and therapeutic efficacy by providing variable contouring, targeted mechanical stimulation, and user-specific comfort enhancements.

The topper modules are designed to be removably affixed to the base platform through mechanical fasteners, magnetic couplings, alignment tracks, or surface friction interfaces. This modularity facilitates rapid substitution of toppers, allowing the system to be reconfigured for distinct therapeutic use cases, anatomical profiles, or environmental conditions.

Representative topper modules include, but are not limited to: Air-cell massage toppers, comprising inflatable bladders or zones that cyclically expand and contract to produce compression waveforms across the user's body; Shiatsu roller modules, incorporating embedded mechanical actuators configured to provide kneading, rolling, or percussive stimulation to localized tissue regions; Passive comfort layers, including memory foam, viscoelastic polymers, or gel-infused cushions, designed to distribute pressure evenly and maintain vibrational coupling efficiency.

Each topper module is selected or configured based on the desired therapeutic outcome, such as deep tissue activation, gentle entrainment, or passive relaxation. Certain embodiments may support layered configurations wherein two or more topper elements are combined (e.g., a passive gel cushion overlying an active roller assembly) to produce compound effects.

The geometric and material properties of the toppers are engineered to maintain consistent transmission of vibrational energy from the underlying transducer elements to the user's body. This includes optimization for acoustic impedance matching, frequency-dependent damping characteristics, and thermal stability.

Additionally, topper modules may be contoured to accommodate variations in body morphology, such as spinal curvature, limb placement, or pressure-sensitive zones. This ergonomic adaptability enhances both comfort and therapeutic precision, minimizing vibrational energy loss due to poor contact or anatomical mismatch.

The ability to interchange topper modules allows practitioners or users to configure the system for a broad spectrum of applications—including clinical neuromodulation, sports recovery, relaxation therapy, and mobility-impaired user support—thereby expanding the functional scope and personalization capacity of the system.

### Modular Configurations and Transducer Deployment

In accordance with various embodiments, the system is configured to permit a wide array of modular configurations of vibroacoustic and sensory-stimulation components, thereby supporting flexible placement, individualized targeting, and rapid reconfiguration of therapy setups. The architecture supports deployment as a therapy table, mat, or other user support surface, with interchangeable and repositionable elements that adapt to differing forms of use and anatomical regions.

Modular Therapy Surface. A primary support surface comprises a base structure designed to accommodate interchangeable topper modules and transducer arrays. The topper modules may be swapped or reconfigured to present distinct therapeutic surfaces—such as air-cell massage assemblies, roller massage mechanisms, gel or foam comfort overlays, or other passive comfort layers-thereby adapting the system for deep-tissue massage, relaxation therapy, neuromodulation entrainment, or postural support.

Repositionable Transducer Mounting. The system includes transducer mounting surfaces, for example, backer boards or cushion inserts integrated within the topper or base structure. These mounts are arranged to receive vibroacoustic actuator elements (i.e., “transducer pucks”) that can be repositioned across the platform surface to allow localized or regional stimulation. The modular nature enables a practitioner or user to target segments such as hands, feet, torso, neck, lumbar spine, or other body zones by relocating transducers accordingly. Embedded transducer arrays spanning larger body zones facilitate coordinated vibration across multiple channels.

Multi-Plane Vibrational Field. Transducer configurations may be arranged both beneath and above the user's body, thereby generating a three-dimensional vibrational “cocoon” effect. The dual-plane arrangement supports immersive full-body stimulation as well as selective regional engagement. This capacity enables the system to envelop the user in coordinated tactile input and to direct energy to both core anatomical zones and peripheral regions such as limbs or lateral surfaces.

Adaptive Transducer Modalities. The system supports multiple modes of transducer orientation and arrangement, enabling rapid transition between gentler, distributed vibrational stimulation and more localized, high-intensity delivery. For example, transducer modules may be flipped, inverted, or re-zipped within cushion assemblies to switch between “puck-in” (softer, diffuse) and “puck-out” (focused, intense) modes. Stacked cushion modules may allow layered combinations of passive and active stimulation. In configurations without topper modules, the system may be deployed directly on alternative surfaces such as beds, furniture, or folded mat forms while retaining functional performance.

Signal and Control Infrastructure. A central control hub is associated with the modular system to coordinate actuators, sensors, and feedback systems. The control unit drives transducer amplitude, frequency, and phase, while also interfacing with biometric sensors and environmental modules. Through the control interface, the operator may monitor user state and adjust stimulation parameters accordingly, and real-time visualization of transducer status and synchronization may be presented.

Integration with Multi-Sensory Elements. The modular configuration supports synchronized delivery of auditory, visual, and tactile stimuli. Optional components, such as a light-shield to reduce ambient visual interference and headphones for synchronized audio, complement the vibroacoustic system. The multi-plane transducer deployment is harmonized with sensory modules to ensure coherent multi-modal entrainment and neuromodulatory engagement.

Versatile Deployment Modes. The configuration supports rapid re-assembly and re-configuration to suit diverse use cases. The system may be deployed as a full therapy table for supine sessions, converted to seated configurations for meditation or stress reduction, or folded for transport or portable applications. Modular components can be swapped or removed without specialized tools, enabling efficient adaptation for home, clinical, or mobile contexts.

### Topper Module Architectures and 3D Cocooning Therapy

In accordance with various embodiments, the system includes interchangeable topper modules that interface mechanically and acoustically with the underlying base platform, enabling layered multi-modal stimulation and a three-dimensional “cocooning” therapeutic envelope. Each topper module is configured to provide distinct therapeutic functionalities and may be employed singularly or in combination to support vibration, compression, stretching, or passive support.

Representative topper modules include, but are not limited to: A module incorporating neck-region rollers and full-body haptic vibrators, offering targeted cervical support combined with distributed vibrational input. A shiatsu-style roller module configured for deep-tissue massage along the spinal region, with adjustable pressure settings to customize user comfort. An air-cell air-wrap/stretch module comprising multiple pneumatic zones, including (a) a hip/leg hug section applying compressive massage to the lower body, (b) a neck traction/stretch zone, and (c) under-back air-cell modules providing adjustable lumbar support and decompression.

Each topper module is selectable and attachable to the base platform and may be layered over or positioned beneath other system components. Moreover, the topper modules may embed vibroacoustic transducer elements or be used in conjunction with sub-lying transducer arrays to amplify vibrational delivery and integrate multiple stimulus modalities.

The system supports a therapy surface configuration whereby the topper module is mounted over the base platform. Beneath or within the topper, vibroacoustic transducer elements are positioned to deliver synchronized vibration from both below and above the user. In operation a physiological sensor continuously monitors biometric indicators—such as heart rate or skin conductance—to enable adaptive stimulation during the session. Localized transducer pucks may be placed under the user to deliver concentrated vibrational energy to selected anatomical zones. The system further incorporates optional acoustic and visual isolation elements including a light-shield and synchronized headphones, as well as visual entrainment systems (e.g., tunable illumination) to complement the vibroacoustic effect.

A wireless and modular control architecture supports the topper module ecosystem. The architecture includes a universal power supply and connector module servicing the topper units, wireless handheld or wearable controllers enabling remote adjustment of topper and transducer functions, and a standardized connector interface for modular component coupling. User input may be provided via manual controls or wireless interfaces to adjust topper behavior and stimulation parameters in real time.

In one embodiment, a cocooning configuration is achieved by draping topper modules both below and above the user body, thereby creating an enveloping stimulation environment combining compression, vibration, and thermal or sensory isolation. Through such layered deployment, the user is immersed in a multi-dimensional therapeutic field that engages multiple sensory channels concurrently.

Layered topper configurations allow modules to be stacked or used individually, thereby enabling targeted therapy of specific body regions (e.g., hip/leg compression paired with neck traction). The system may integrate thermal modulation (heating or cooling) alongside vibration and compression to enhance therapeutic effect. Embedded sensors-including accelerometers and microphones placed across different body regions-facilitate real-time measurement of vibrational coupling, compression dynamics, and energy distribution, enabling closed-loop adjustment of stimulation parameters.

The topper module architecture thus provides a highly configurable, multi-functional layer that enhances the adaptability, precision, and therapeutic depth of the overall vibroacoustic neuromodulation system.

### Adaptive Configurations and Deployment Modes

In accordance with various embodiments, the therapy system is configured to adopt multiple physical configurations and form-factors to suit diverse therapeutic, wellness, mobile, or environmental deployment contexts. The system's modular architecture enables rapid transition between full-scale installation, compact portable formats, and specialized surface overlays, all while preserving synchronised multisensory neuromodulation functionality.

Representative configurations include: Full-Setup Configuration: A therapy table or full-length platform is overlaid with a modular topper and modular transducer cushion assembly. Complementary accessories such as auditory delivery devices (headphones), ambient light-shielding structures, and immersive display modules (tablet interface, projector, AR/VR headset) are integrated to provide combined vibroacoustic, auditory, visual and biometric feedback. Elastic Cover Configuration: In this form, a stretchable or elastic cover is deployed to secure interchangeable topper modules onto the base platform. The cover may also provide a grounded or conductive function. Control devices (tablet, projector) remain incorporated into the configuration, enabling full functional control while adapting to a simplified physical form. Weighted Blanket Configuration: A weighted-cover weave is used to hold modular cushions and topper elements in place via weight and tension. The system incorporates the control interface and immersive modules to support synchronized sensory output in a configuration optimized for comfort and passive therapy environments. Foldable Configuration: The base platform incorporates hinge or fold-axis mechanisms enabling collapse or fold-down for storage or transport without sacrificing functional integrity of embedded transducer components. The foldable format supports compact deployment while maintaining full vibroacoustic, auditory, visual, and control system capabilities. Bottom or Underside View Configuration: From an underside perspective, the structure reveals multiple transducer mounting points, quick-attach reinforcement cables between supports, and additional structural elements to maintain load-bearing capacity and rigidity during use across configurations. Multiple Setup Options: Alternative geometries and deployment modes are supported, including wide versus slim table formats, folded transducer cushions with reconfigurable modes, variable width flat configurations, raised-back support for floor or alternative surface use, and slim or wide portable table modules that fold for transport.

This versatility permits the platform to be adjusted according to environmental constraints, user posture preferences, therapy modality, and space availability. All configurations maintain functional integration of vibroacoustic actuators, auditory and visual stimulation modules, biometric sensors, and control interfaces to ensure consistent multi-modal neuromodulation across deployment formats.

Thus, the adaptive configuration capability enhances the system's applicability across home, clinical, research, mobile, group, and individual use cases, providing flexibility, scalability, personalization, and consistent therapeutic efficacy.

### Integrated Control, Cushion Configurations, and Adjustability

In accordance with various embodiments, the system further comprises an integrated control and adjustable cushion architecture that enables adaptive configuration of therapy surfaces, sensory modules, and actuation parameters. This architecture facilitates rapid re-configuration of therapeutic setups and supports personalization of stimulation modalities across different use cases (e.g., full-body therapy, seated meditation, portable deployment).

### Control and Adjustment Features

The system includes a primary user or clinician control interface configured to present real-time feedback of physiologic and session variables and to allow dynamic adjustment of such parameters. The control interface permits modification of therapy settings including amplitude, frequency, phase offsets, timing, module selection, and stimulation modality.

An example configuration incorporates an adjustable topper module providing pneumatic cocooning massage functionality, enabling wrap-around compression and vibroacoustic actuation. Adjacent modules include mechanical massage mechanisms (such as roller assemblies) configured for kneading along the spinal region. An elevated back support configuration may be provided to accommodate reclined postures; in one embodiment, a pillow accessory includes a thermal element powered via a universal receptacle integrated into the topper architecture, thereby enabling combined vibrational, compressive, and thermal stimulation.

“Temperature management implements a dual-exponential predictor with a 42° C. cutoff; a watchdog and analog cutoff relay force amplifier low-power on threshold or controller fault.”

Seated Meditation Option with Folded Support Surface. In certain embodiments, the system supports a folded-table variant optimized for seated meditation or cross-legged user postures. The foldable configuration allows the therapy surface to transform from a full-lie platform to a compact seated module, while retaining full access to modular cushion and transducer assemblies and the integrated control system.

Connection System and Transducer “Sandwich” Architecture. The adjustable cushion architecture includes modular cushion assemblies arranged in a “sandwich” configuration, comprising a central cushion or topper layer flanked above and/or below by transducer modules. This bidirectional layering enables controlled delivery of vibrational energy from both directions relative to the user's body. The cushion modules are designed to integrate seamlessly with topper modules, facilitating combinations of pneumatic compression, mechanical massage, and vibroacoustic actuation. A universal power and control connector enables rapid swapping of modules-including transducers, toppers, and control units-without requiring system disassembly or recalibration, thereby supporting agile therapy customization and maintenance.

### Cushion and Transducer Configuration Architecture

In accordance with various embodiments, the therapy system comprises modular cushion assemblies configured to support configurable transducer placements, thereby enabling flexible spatial, frequency, and amplitude control of vibroacoustic stimulation tailored to individual anatomical regions and therapeutic objectives.

### Cushion Module Without Transducer

A first configuration of the cushion assembly comprises a cushion element that may be provided with or without an external cover. The internal cavity of the cushion is dimensioned to receive one or more transducer modules or other therapeutic inserts. The outer cover may incorporate zippered or elastic closures, permitting rapid access to the interior cavity for insertion, removal, repositioning, or reorientation of transducer elements or therapeutic cartridges without disassembly of the underlying support structure.

Single Transducer Pad with Mounting Backboard

In another configuration, a single transducer pad is secured to a rigid backer board designed for placement within the cushion module. This arrangement provides a stable and repeatable mechanical interface between the pad and the user's body, thereby ensuring consistent physical coupling and accurate vibrational energy transfer to a targeted anatomical region.

### Multiple Transducer Configuration on a Shared Backer Board Under Cover

A further configuration comprises multiple vibroacoustic transducer units affixed to a shared rigid backer board, which is then enclosed beneath the cushion module's cover. This layout supports synchronized multi-point stimulation across a broader anatomical region, facilitating entrainment or neuromodulation across multiple body zones concurrently. The shared backer board ensures alignment and stable mounting of multiple actuators while preserving flexibility for distributed patterns and relatively diffuse vibrational coverage.

### Dual Transducer Pad Configuration on Isolated Backer Boards

In still further embodiments, two transducer pads are each mounted on separate rigid backer boards, allowing these pads to operate independently or in synchrony. This architecture supports advanced stimulation paradigms—such as alternating pulses between pads, lateralized entrainment across paired anatomical regions, or region-specific neuromodulatory protocols. Each backer board may be calibrated independently for frequency, amplitude, phase offset, or waveform shape, thereby enabling targeted control of differential stimulation across the body.

### Multi-Point Stimulation Using Independent Transducers

In yet further embodiments, multiple independent transducer modules are distributed across the cushion assembly to engage distinct anatomical regions each with customizable frequency, amplitude, and phase. Individual modules may be operated at different frequencies or amplitudes within the same treatment session, enabling intricate entrainment strategies, asymmetrical neuromodulation, and responsive feedback protocols tailored to complex therapeutic or research objectives.

Collectively, the cushion and transducer configuration architecture provides a highly configurable and adaptive interface for vibroacoustic energy delivery. By enabling rapid reconfiguration of transducer placement, module orientation, and actuator coupling, the system supports personalized therapy across diverse body types, treatment goals, and use-case environments.

### Multi-Sensory Stimulation Subsystems

Vibroacoustic Transducers. In accordance with various embodiments, the system comprises one or more vibroacoustic transducer assemblies configured to deliver mechanically coupled vibrational stimuli to a user's body via intermediary structural layers such as cushions, mats, or topper modules. These transducer assemblies are adapted to generate vibrational energy across defined frequency and amplitude ranges suitable for neuromodulation, relaxation, stimulation, or entrainment applications.

Each vibroacoustic transducer includes a motor, driver coil, or piezoelectric element housed within a modular casing or puck. The casing may be composed of rigid or semi-rigid materials optimized for mechanical resonance, thermal management, and vibration containment. Transducer units are physically coupled to the user through intermediate transmission media—such as high-density foam, thermoplastic composites, or elastomeric layers—configured to maximize vibrational transfer efficiency while minimizing signal attenuation or acoustic leakage.

The transducers are mechanically mounted into designated receptacles or modular inserts within foldable mats, topper modules, or cushion systems. Mounting techniques may include magnetic coupling, snap-in mechanisms, zippered enclosures, or mechanically indexed housings that ensure consistent alignment and physical coupling. The modularity of the mounting system enables repositioning or reconfiguration of transducer locations to accommodate diverse user anatomies or targeted therapeutic zones.

Each transducer is operable across a specified frequency spectrum, typically ranging from sub-audible infrasonic bands (e.g., 5-20 Hz) up through low-to-mid auditory frequencies (e.g., 20-500 Hz). Frequency output may be individually addressable per transducer unit and modulated in real time via digital or analog control signals. The amplitude of vibrational output is similarly adjustable, either via pulse-width modulation, analog gain control, or other means, allowing the system to vary intensity based on therapeutic protocol, user feedback, or biometric inputs.

Vibroacoustic output is generated through controlled oscillation of the transducer element, which transmits mechanical energy through the mat or topper structure to the user's body. In some embodiments, transducers are embedded above, below, or around the user to create a three-dimensional vibrational field or “cocoon,” enhancing the immersive quality and efficacy of the therapy.

In certain configurations, dual-mode transducers are employed to provide both audible sound and sub-audible vibration through a unified transducer assembly. Phase, latency, and frequency synchronization between transducer units is governed by a central control module or distributed synchronization system to ensure coherent delivery of entrainment signals.

In advanced embodiments, the transducers may be dynamically modulated based on real-time biometric feedback—such as heart rate variability, galvanic skin response, or brainwave entrainment metrics—allowing closed-loop adaptation of frequency, waveform, or amplitude to optimize therapeutic outcomes.

This transducer architecture provides the foundational mechanism for delivering precisely controlled, high-fidelity vibrational stimuli across varied user configurations and treatment environments, supporting a wide range of neuromodulatory and sensory-integrated therapeutic protocols.

Auditory and Visual Modules. In accordance with various embodiments, the system further comprises one or more auditory and visual stimulus modules, optionally integrated with sensory deprivation mechanisms, configured to enhance neuromodulatory outcomes through synchronized multi-sensory entrainment. The architecture supports both reduction of environmental interference and delivery of time-locked sensory signals aligned with vibrational input and neural feedback parameters.

The system includes sensory deprivation modules operable to attenuate external sensory stimuli. These modules may comprise physical light shields, acoustic damping enclosures, noise-isolating ear covers, visual occlusion goggles, or soft-fabric enclosures configured to reduce exposure to ambient light, sound, and tactile inputs. The purpose of these components is to establish a minimally stimulated environment that promotes introspective focus and enhances receptivity to internalized sensory signals.

In parallel, the system delivers controlled sensory stimulation via integrated auditory and visual output devices. Auditory stimuli are delivered through high-fidelity headphones, embedded transducer speakers, or bone-conduction elements configured to output frequency-specific sound waves. These signals may include pure tones, modulated waveforms, binaural beats, or audio tracks designed to promote neural entrainment, cognitive arousal, relaxation, or other target states. Sound delivery is precisely synchronized with vibroacoustic output using latency correction, phase alignment, and time-domain signal processing.

Visual stimulation modules may include LED arrays, infrared or near-infrared light sources, and/or augmented reality (AR) or virtual reality (VR) display systems. These devices are operable to emit pulsed or modulated visual stimuli, such as stroboscopic flashes or rhythmic light sequences, designed to drive or disrupt visual cortex oscillations in alignment with target neural frequencies. In advanced embodiments, visual patterns are dynamically modulated based on real-time biometric or neurofeedback data to optimize cognitive engagement or brainwave entrainment.

Synchronization across auditory, visual, and vibrational outputs is achieved through a central control module or distributed synchronization network that compensates for inherent latency differences across modalities. Calibration systems comprising accelerometers, microphones, photodiodes, and other latency sensing components may be employed to verify and maintain alignment within defined timing tolerances. This ensures that all sensory modalities operate in coherent temporal and spectral alignment, maximizing the system's entrainment potential.

The combination of sensory deprivation and precisely coordinated multi-modal stimulation provides a configurable and immersive therapeutic platform capable of modulating cognitive, emotional, and physiological states with high specificity.

### 3D Vibrational Enclosure (Perimeter and Over Body Modules)

In accordance with various embodiments, the system supports a three-dimensional vibroacoustic enclosure configured to envelop the user with vibratory, auditory and visual stimuli while reducing ambient sensory interference, thereby enhancing neuromodulatory depth, entrainment efficacy and autonomic stabilization.

### Perimeter Modules

In certain configurations, the system includes raised perimeter structures composed of resilient, conforming materials such as foam, memory-foam, or gel, arranged to create a recessed cavity or containment zone into which the user partially settles. The perimeter structures may be mounted at the edges of the base platform or attached to foldable mat configurations via removable fasteners (e.g., hook-and-loop, embedded magnets, or connector sleeves). Vibroacoustic transducer units are embedded within or alongside these perimeter modules, thereby delivering lateral vibrational input to the user's flanks, ribs, or torso midline. This lateral stimulation complements vertical input from under-body transducers and supports mid-line entrainment protocols relevant to vagal nerve tone, intercostal activation, or fascial modulation. In one embodiment, the perimeter transducers are synchronized in timing and phase with under-body transducers to maintain coherent spatio-temporal delivery of vibrational energy across the enclosure.

### Over-Body Modules

The enclosure may further comprise one or more over-body layers positioned above the user's torso or extremities. These layers may be constructed as flexible honeycomb gel cushions, mesh-fabric overlays, shaped foam structures, or supported by lightweight arching frames. Vibroacoustic transducers are integrated into or positioned behind these over-body modules to deliver downward or inward vibrational input. Optional features include transducer pucks repositionable within modular overlays (e.g., mesh pockets, magnetic arrays, or defined cut-out slots) to granulate vibrational delivery across anterior or lateral body zones. This multi-directional vibrational field enables constructive interference, spatial energy shaping, and targeted neuromodulation of specific anatomical zones or neural pathways.

### Integrated 3D Immersion

When under-body, perimeter, and over-body modules are combined, the system creates a fully enclosed three-dimensional vibrational field. This configuration enables synchronized, spatially distributed stimulation that closely mirrors the user's somatosensory map, thereby facilitating immersive neurosensory protocols. The combined enclosure supports deep internal sensation, heightened tactile perception, and focused entertainment of neural networks, resulting in improved outcomes for stress reduction, cognitive focus, sensory integration, or neuromodulation therapies.

The three-dimensional vibrational enclosure enhances both perceptual intensity of stimulation and neuromodulatory efficacy compared to traditional single-plane mat systems. It is particularly suited for applications requiring full-body immersion, multi-modal feedback integration, or advanced entrainment schemes involving vibration, sound, light, and neural feedback.

### Biometric Integration and EEG-Guided Adaptation

High-Density EEG System Design. In accordance with various embodiments, the system incorporates a high-density electroencephalography (HD EEG) subsystem configured to enable high-resolution, real-time brain activity mapping for adaptive neuromodulation. The integration of the HD EEG subsystem allows the system to align multi-sensory stimulation—including vibroacoustic, auditory, and visual modalities—with the user's neural state as it evolves in real time, thereby enhancing therapeutic precision, personalization, and reproducibility of outcomes.

A. HD EEG Cap Architecture and Electrode Configuration. The HD EEG subsystem comprises an electrode cap configured with up to 280 channels distributed across the scalp to achieve dense spatial sampling of cortical activity. The electrode array is arranged to capture neural oscillations across multiple frequency bands—delta, theta, alpha, beta, and gamma—enabling fine-grained mapping of both global and localized brain activity. The electrode layout supports multi-regional acquisition for functional connectivity analysis, source localization, and biomarker identification.

Each electrode is designed to maintain uniform contact pressure and impedance stability across the scalp. Contact elements may employ conductive gel, hydrogel, or dry polymer interfaces to optimize signal quality while minimizing user discomfort. The cap material is flexible, lightweight, and ergonomically contoured to ensure consistent electrode positioning during extended sessions. The system is engineered for rapid setup—allowing full application in under five minutes—thereby supporting clinical, research, and consumer wellness environments that require fast turnaround or multi-user throughput.

Signal Acquisition, Processing, and Control Interface. The HD EEG system interfaces directly with the system's control electronics via one or more amplifier and data-acquisition units. Each channel is digitized at high sampling rates to preserve spectral fidelity and temporal resolution. The control interface—accessible through a tablet, computer, or dedicated console—serves as the operator's central hub for configuring session parameters, monitoring real-time EEG traces, and performing quality control.

The interface enables adjustment of acquisition parameters such as sampling frequency, channel selection, reference configuration, and frequency-band filtering. Built-in diagnostics display electrode impedance in real time, highlighting channels that require recalibration. Automated correction algorithms may be invoked to compensate for signal drift or impedance imbalance. The system's graphical interface presents dynamic topographic maps, frequency-domain visualizations, and event-related synchronization metrics, allowing practitioners to observe and respond to changes in neural state as they occur.

Integration with Multisensory and Biometric Systems. The HD EEG subsystem is synchronized with the vibroacoustic, auditory, and visual modules of the system to form a closed-loop neuromodulation architecture. The control electronics receive EEG-derived data streams—including frequency-band power, phase coherence, and connectivity metrics—and adjust the timing, amplitude, and frequency of vibroacoustic and audiovisual stimuli accordingly.

In one embodiment, when gamma-band activity in the prefrontal cortex falls below a target threshold, the system dynamically generates a vibroacoustic signal tuned to the user's individualized gamma frequency to promote re-entrainment. Conversely, excessive coherence in a given band may trigger desynchronizing stimuli for neural resetting. These feedback mechanisms allow continuous, adaptive modulation of sensory input based on detected brain activity.

Rapid Brain-Mapping Kiosk Integration. In another embodiment, the HD EEG subsystem is incorporated into a Rapid Brain Mapping Kiosk configured for high-throughput assessment and individualized calibration. The kiosk includes an ergonomic base that supports the participant during mapping sessions and integrates HD EEG sensors, vibroacoustic transducers, spatial imaging systems, and biometric monitors into a unified structure.

Spatial cameras capture three-dimensional models of the user's head and body geometry to guide electrode alignment and ensure precise sensor placement. Physiological sensors-including heart-rate and galvanic-skin-response monitors-collect biometric data concurrent with EEG acquisition. Microphones and accelerometers are used to capture vibroacoustic feedback and measure mechanical coupling between stimulus and body response. A synchronization module with optical sensors and timing markers ensures phase-locked alignment between visual, auditory, and vibrational outputs.

The kiosk's control interface manages data capture, visualization, and calibration. Successive neuromap snapshots are collected over time (e.g., t₁, t₂, t₃) to track longitudinal changes in brain activity. The system analyzes frequency-band power shifts, latency variations, and coherence metrics to generate individualized change reports, which inform future entrainment or neuromodulation protocols.

Performance Characteristics and Applications. The HD EEG subsystem combines speed, precision, and scalability, supporting both individualized therapy and large-scale research. High-density coverage allows source-space reconstruction and three-dimensional modeling of cortical and subcortical neural activity. The system's high signal-to-noise ratio, artifact suppression, and automated impedance management enable consistent performance across users and sessions.

This architecture is suitable for: Personalized neuromodulation and entrainment sessions requiring real-time brain-state detection; Clinical and research applications involving source-localized EEG analysis and biomarker discovery; Population-level neural data collection via distributed kiosk networks; and Closed-loop neuroadaptive therapies that integrate vibroacoustic, auditory, and visual feedback based on instantaneous EEG response.

By combining rapid electrode deployment, high-resolution signal acquisition, and real-time feedback integration, the HD EEG subsystem provides a scalable foundation for precision brain monitoring, adaptive neuromodulation, and population-level neural analytics.

### Real-Time Neural Feedback Loop

### Personalization of Therapy

Data acquired from the HD EEG system is analyzed in real time and used to inform the system's neuromodulation protocols in several key ways: Identification of neural biomarkers: The EEG signal is analyzed to detect user-specific neural signatures, such as dominant frequency bands, hemispheric imbalances, or cortical hotspots that correspond to stress, focus, fatigue, or emotional states. These biomarkers guide the selection of optimal stimulation frequencies and spatial targeting parameters. Customization of sensory stimuli: Based on the EEG data, the system dynamically adjusts the parameters of vibrational, auditory, and visual stimuli, including:
1. Carrier frequency and waveform shape.
2. Spatial distribution and amplitude of transducer output.
3. Timing offsets to enhance cross-modal coherence and neural resonance.

Monitoring of therapeutic progress: Longitudinal EEG data is used to track changes in brain activity over time. This allows for:
1. Assessment of therapy efficacy based on shifts in frequency dominance, coherence, or amplitude.
2. Adjustment of protocols based on cumulative neurophysiological response.
3. Generation of session reports and biometric dashboards for clinicians, researchers, or users.

This EEG-driven integration enables the system to operate as a closed-loop neuromodulation platform, delivering personalized, adaptive, and quantifiable sensory-based therapy. By aligning stimulus delivery with real-time brain state measurements, the system maximizes therapeutic precision while enabling feedback-informed adjustments throughout each session.

### Real-Time Neural Feedback Loop

In accordance with various embodiments, the system provides a closed-loop neuromodulation architecture in which data acquired from a high-density electroencephalography (HD EEG) subsystem is processed in real time to inform, calibrate, and adapt sensory stimulus parameters. This feedback-driven system enables dynamic personalization of therapeutic protocols based on detected neural biomarkers, instantaneous brain state, and longitudinal trends in neurophysiological response.

### Identification of Neural Biomarkers

The real-time EEG signal is subjected to continuous analysis using spectral, temporal, and spatial algorithms to detect individualized neural signatures. These may include, but are not limited to: Dominant frequency band identification (e.g., alpha, beta, gamma peaks). Hemispheric power asymmetries, indicative of lateralized functional imbalances. Cortical zone activation patterns corresponding to states of focus, fatigue, stress, relaxation, or emotional arousal. Temporal coherence metrics and phase synchronization indicators across spatially distributed electrode regions.

These biomarker metrics are used to define individualized neural profiles, which in turn guide the selection and adjustment of neuromodulatory stimulus parameters to maximize alignment between therapeutic input and brain-state receptivity.

### Customization of Sensory Stimuli Based on EEG Data

The system's control architecture includes algorithms configured to modify vibroacoustic, auditory, and visual stimulus output in direct response to real-time EEG measurements. Adjustments may include: Carrier Frequency and Waveform Shape: The fundamental frequency and harmonic content of vibroacoustic signals are tuned to match the user's current or target neural frequency band (e.g., theta or gamma entrainment). Spatial Distribution and Amplitude Modulation: Transducer outputs are selectively modulated to emphasize specific body regions in correspondence with cortical zones exhibiting biomarker activation. Timing and Phase Offsets: Cross-modal synchronization between auditory, vibrational, and visual stimuli is dynamically adjusted to enhance multimodal coherence and support resonance-based entrainment strategies.

These real-time adjustments allow the system to deliver stimuli in a manner that maximizes neural receptivity, enhances entrainment efficiency, and avoids overstimulation.

### Monitoring of Therapeutic Progress

The system also supports longitudinal tracking of EEG-derived biomarkers, enabling monitoring of session-by-session therapeutic impact and long-term neurophysiological change. This includes: Assessment of Frequency Dynamics: Tracking changes in the prominence of specific frequency bands over time to assess entrainment or desynchronization efficacy. Coherence and Amplitude Modulation Metrics: Measuring shifts in inter-regional synchrony or signal magnitude as indicators of functional adaptation. Automated Report Generation: The system compiles session-specific and cumulative metrics into visual dashboards and summary reports accessible by clinicians, researchers, or users.

This closed-loop configuration enables both intra-session and inter-session personalization, allowing the neuromodulation platform to adapt continuously to the evolving needs and responses of each individual.

### Adaptive Closed-Loop Control System

By integrating high-resolution EEG acquisition with real-time stimulus control, the system operates as an adaptive, EEG-driven neuromodulation platform. Stimulus parameters are continuously recalibrated based on measured neural activity, enabling: Fine-grained, real-time adaptation of stimulation. Consistent alignment between sensory input and current neural state. Dynamic therapy protocols tailored to the user's unique physiological and cognitive profile.

This integration maximizes therapeutic efficacy and safety while enabling quantifiable tracking and validation of outcomes, providing a robust foundation for both individualized treatment and scalable deployment in clinical, wellness, and research contexts.

### Adaptive Modulation of Stimuli

In accordance with various embodiments, the system is configured to modulate vibroacoustic, auditory, and visual stimuli in real time based on biometric and neurophysiological feedback, latency correction, phase alignment, and individual user-specific customization. This adaptive control logic enables personalized neuromodulation by continuously adjusting stimulus parameters in response to the user's dynamic physiological state.

### Feedback-Driven Stimulus Modulation

The control architecture processes real-time data acquired from sensors including high-density electroencephalography (HD EEG), heart-rate monitors, galvanic skin-response sensors, respiratory monitors, accelerometers, microphones, and photonic sensors. The system analyzes these data streams to identify neural biomarkers indicative of states such as stress, fatigue, cognitive focus, emotional arousal, or recovery. Examples of such biomarkers include shifts in gamma, alpha, and theta band power; changes in neural synchrony and phase coherence; response latencies; amplitude variations; and patterns of evoked potentials.

Based on these biomarker signals, the system dynamically adjusts stimulus delivery by controlling one or more of the following parameters: Vibrational Frequency: Transducer output is modulated to match a dominant EEG frequency or a targeted entrainment band (e.g., gamma ~30-50 Hz, alpha ~8-12 Hz, theta ~4-8 Hz, or slow-wave ~0.5-2 Hz). Amplitude and Waveform Shape: The intensity of vibroacoustic, auditory, and visual outputs is adjusted in real time to reflect the user's neural responsiveness, comfort thresholds, or entrainment progression. Spatial Distribution of Stimulation: The system may shift or redistribute vibrational energy to prioritize body-zones aligned with neural signal focal points or latency windows—e.g., increasing stimulation at hands or feet when corresponding cortical rhythms indicate readiness. Timing Offsets and Phase Alignment: Cross-modal coherence is enhanced by adjusting latency and phase offsets among vibroacoustic, auditory, and visual channels, thereby maximizing temporal alignment with neural oscillations and optimizing resonance and entrainment.

### Individual Mapping and Change Detection

The system supports a temporal neuromapping protocol in which successive EEG snapshots are acquired at defined intervals—such as baseline, mid-session, and post-session. The system compares these snapshots to detect longitudinal changes in neural dynamics: for example, shifts in gamma/alpha/theta power; variation in phase coherence; changes in neural synchrony; fluctuations in response latency or amplitude; and evolving evoked-potential patterns. The outcome is an individual change report indicating neurophysiological adaptations, zones of sensitivity, or required protocol adjustment. The report is used to tailor future therapy sessions, optimize neuromodulation parameters, and improve session efficiency and personalization.

Closed-Loop Control and Real-Time Adaptation. By integrating the HD EEG subsystem, biometric sensors, and stimulus modules into a unified feedback architecture, the system operates as a closed-loop neuromodulation platform. Real-time neural-state detection triggers immediate stimulus modulation: for example, detection of deep-sleep slow-wave activity may prompt the system to initiate low-frequency vibration (e.g., 0.5-2 Hz); conversely, during meditation the system may trigger gamma or alpha stimulation when EEG indicates readiness. The control logic references stored user-specific EEG profiles—including baseline frequencies, reaction to prior entrainment stimuli, and session history—to deliver personalized stimulus protocols. Over time the system detects shifts in neural patterns associated with stress, fatigue, or recovery and adapts protocols accordingly.

This architecture allows alignment of stimulus delivery with the user's moment-to-moment neurological state, thereby maximizing therapeutic precision, promoting entrainment or desynchronization as appropriate, and enabling quantifiable tracking of outcomes and longitudinal neural change.

### Rapid Brain Mapping Kiosk and Individual Profiling

In accordance with various embodiments, the system includes a compact, integrated kiosk architecture configured for high-throughput individual neuromodulatory profiling and personalized therapy modulation. The kiosk supports real-time neural and physiological data acquisition, synchronized multi-sensory stimulus delivery, and longitudinal tracking of user brain-body state to guide adaptive protocol optimization.

### Individual Brain Mapping Configuration

The kiosk comprises a dedicated user station configured to create a controlled, sensor-rich environment for capturing high-resolution neural and physiological data with minimal environmental interference. Key components include:

A modular kiosk structure providing structural support, integrated wiring, and user ergonomics designed for neural mapping and therapeutic session use.

A control interface, such as a tablet or computer terminal, enabling session configuration, real-time visualization of EEG and biometric data, dynamic adjustment of stimulus settings, access to stored user profiles, and operator oversight.

A high-density electroencephalography (HD EEG) subsystem comprising up to 280 electrodes distributed across the scalp to provide dense spatial and temporal sampling of cortical and sub-cortical oscillatory activity. The system supports acquisition of neural signals across major frequency bands (delta, theta, alpha, beta, gamma), enabling source localization, connectivity modeling, and entrainment analysis.

An amplifier/controller unit that processes EEG input, coordinates signal routing to vibroacoustic transducers and sensory modules, manages latency and jitter, supports phase alignment across channels, and enables multi-channel modulation of stimuli based on neural responsiveness.

Microphones and accelerometers incorporated into the kiosk or handheld calibration devices to capture acoustic output, vibrational feedback, and mechanical coupling metrics. These sensors enable verification of somatosensory engagement, measurement of waveform integrity, spatial fidelity of vibrational delivery, and adjustment of stimulus characteristics in real time.

Spatial imaging cameras that capture three-dimensional head and body geometry of the user, facilitating accurate alignment of EEG sensors, transducer placement, motion compensation, and anatomical registration between sessions and participants.

Physiological sensors—including galvanic skin response (GSR), heart rate monitors, respiratory or temperature sensors—that continuously monitor biometric state, inform stimulus adaptation, and support safety and state-based protocols.

A support stand or ergonomic mounting structure that positions the control interface and monitoring displays adjacent to the user in optimized alignment for both practitioner and user accessibility.

A calibration device or module that measures vibrational amplitude, transducer output, and audio intensity at defined body regions, thereby verifying accurate and consistent energy delivery across sessions and anatomical zones.

Synchronization modules including optical/photo sensors, vibration sensors, and timing markers that verify precise temporal alignment of stimulus emissions across modalities and ensure phase-locked delivery of vibroacoustic, auditory, and visual stimuli in coordination with neural and biometric feedback.

A central synchronization control system that orchestrates stimulus timing, compensates for latency and jitter across sensor and actuator channels, aligns EEG event markers with stimulus triggers, and supports multi-sensory coordination in a closed-loop architecture.

### Individual Mapping and Change Detection

In this configuration, the system implements a temporal neuromapping protocol in which multiple EEG “snapshots” are captured at defined intervals (for example, baseline, mid-session, post-session) to represent the user's brain state under varying conditions or therapy stages. The system performs analysis of key neural dynamics including, but not limited to: Shifts in gamma, alpha, and theta band power; changes in neural synchrony and phase coherence; variations in response latency, amplitude, and evoked potential patterns.

Based on this longitudinal data, the system generates an individual change report that compares neuromapping metrics over time and identifies neurophysiological adaptations, zones of sensitivity, improvement or required adjustment. The results of the change report guide subsequent therapy sessions by: Tailoring stimulation protocols to individual neural response profiles; optimizing neuromodulation parameters for improved alignment with user biomarkers; enhancing session efficiency, personalization, and outcome predictability.

This kiosk-based mapping and profiling platform enables scalable, data-driven personalization of vibroacoustic and multi-sensory therapy, providing precise neural tracking capabilities and functioning as the diagnostic and analytic backbone of the broader modular neuromodulation system.

### Population Brain Mapping Network and Protocol Optimization

In yet another embodiment, the system is extended to operate as a networked platform for population-scale brain mapping and multi-user neuromodulatory protocol optimization. This configuration enables the collection, analysis, and comparison of high-resolution neural and biometric data across large cohorts, supporting the development of adaptive therapy models and population-informed stimulation protocols.

### Networked Architecture for Multi-User Profiling

The system comprises a distributed network of brain mapping kiosks or modular therapy systems, each equipped with high-density EEG acquisition, multi-modal stimulus delivery, and real-time biometric monitoring. These nodes are connected via secure data transmission infrastructure (e.g., encrypted local network or cloud-based architecture), allowing centralized data aggregation, processing, and protocol optimization across sessions and users.

Each system node captures and transmits the following: High-resolution EEG data spanning up to 280 channels per user, including frequency band segmentation, coherence metrics, and regional activation patterns. Biometric sensor data, including heart rate variability, galvanic skin response, respiratory rhythm, and other physiological indicators of autonomic state. Stimulus delivery logs, documenting timing, amplitude, waveform, and spatial distribution of vibroacoustic, auditory, and visual inputs. User configuration parameters, including demographic data (age, sex, baseline neuroprofile), therapy goals, session type, and feedback outcomes.

### Population-Level Analytics and Adaptive Protocol Generation

The aggregated data is analyzed using a centralized neural analytics engine, which employs statistical modeling, machine learning algorithms, and biomarker clustering techniques to identify population-level patterns in neural dynamics, therapy responsiveness, and optimal stimulation parameters.

This analysis supports the following capabilities: Identification of cross-user neurophysiological archetypes and classification into responder types based on EEG and biometric profiles. Discovery of optimal entrainment protocols for specific subgroups, including frequency ranges, waveform shapes, amplitude envelopes, and multi-modal synchronization strategies. Longitudinal tracking of changes in individual and cohort neural responses over time, enabling detection of trends, therapy efficacy, and regression or adaptation patterns.

The resulting models inform protocol optimization through: Preloaded stimulation templates tailored to different cognitive, emotional, or physiological goals (e.g., stress reduction, focus enhancement, sleep modulation). Adaptive therapy recommendations based on real-time comparison of a user's neural state with population-derived targets. Predictive modeling of therapeutic outcomes based on historical performance of similar neurotypes under specific stimulation conditions.

### Clinical and Research Applications

This population brain mapping network supports diverse use cases, including: Scalable deployment in wellness centers, clinics, or group therapy settings to provide individualized care informed by collective neural data. Longitudinal studies on brain plasticity, recovery, or cognitive training by tracking multi-user neurometrics across extended periods. Data-driven refinement of vibroacoustic, auditory, and visual entrainment paradigms based on broad-spectrum physiological and neurological feedback.

The networked configuration transforms the modular vibroacoustic therapy platform from an individualized therapeutic tool into an integrated system for scalable neuromodulatory research, cross-user optimization, and evidence-based therapy design.

### Latency Mapping and Phase Synchronization

Latency Measurement Mechanisms and Phase-Aligned Multi-Modal Synchronization. In accordance with further embodiments, the system provides a mechanism for detecting and compensating for individual neural and perceptual response latencies across multiple sensory modalities-specifically, vibroacoustic, auditory, and visual inputs. The system enables phase-aligned, temporally synchronized stimulus delivery, thereby enhancing the coherence and efficacy of multi-modal neuromodulation protocols.

### Latency Detection and Multi-Modal Stimulus Timing

The system comprises a plurality of stimulus delivery devices and feedback sensors configured to measure sensory processing latencies in real time. These include: Stimulus Generation Modules: Vibroacoustic transducers, auditory output devices (e.g., headphones or speakers), and visual stimulus emitters (e.g., LEDs, strobing displays, or AR/VR modules) are configured to deliver temporally controlled, parameter-defined stimuli to the user. Sensor Feedback Modules: Sensors including accelerometers, microphones, photocells, and high-density electroencephalography (HD EEG) interfaces are strategically positioned to detect vibrational onset, acoustic emission, visual stimulus timing, and the corresponding physiological or neural responses.

This sensor network is operable to: Measure intermodal latency differentials (e.g., auditory vs. tactile perception latency). Assess intra-body transmission delay variances (e.g., vibration propagation delay from torso to extremities). Track latency dynamics across session time (e.g., variations due to cognitive state, fatigue, or adaptation).

### Real-Time Latency Mapping and Synchronization

The system performs latency mapping either during an initial calibration phase or dynamically throughout therapy. Real-time data from neural and biometric sensors is analyzed by a latency engine configured to: Calculate user-specific response times across modalities. Detect phase misalignment or drift during ongoing multi-modal stimulation. Adaptively correct temporal offsets between stimulus initiation and neural entrainment windows.

The system compensates for these latencies by applying calculated phase shifts to individual stimulus outputs, ensuring that auditory, vibrational, and visual stimuli are temporally aligned when received at the cortical and perceptual level.

### Redundant Synchronization Framework

To ensure precision and robustness, the system implements a redundant synchronization architecture, comprising: Photonic Sensors (e.g., photocells 126): Detect visual stimulus onset to verify temporal output from LEDs or display modules. Vibration Sensors (e.g., accelerometers 130.1): Embedded in transducer modules or handheld calibration tools to measure onset and waveform characteristics of vibrational output. Acoustic Sensors (e.g., microphones 130.2): Record the emission of auditory signals to verify waveform integrity and transmission latency. Synchronization Processor (e.g., sync module 128): Aggregates and evaluates sensor data to compute corrections for latency, jitter, or phase drift. The system applies compensatory adjustments to maintain phase-locked multi-modal delivery.

### Adaptive Latency and Calibration Algorithms

Advanced algorithms enable real-time adaptation of stimulus timing in response to dynamic user states. These algorithms: Continuously monitor EEG signals and biometric parameters (e.g., heart rate variability, GSR). Detect shifts in neural response latency or desynchronization due to user-specific conditions (e.g., arousal level, attention shifts). Automatically adjust the temporal profile of each modality (e.g., delay, waveform shape, amplitude) to maintain cross-modal phase coherence.

### Manual and Sensor-Driven Calibration Tools

The system optionally includes a handheld calibration module (112.5) incorporating integrated accelerometers and microphones to: Measure vibrational amplitude and latency at various anatomical sites. Analyze spectral changes induced by user vocalization (e.g., humming) to infer depth of vibrational penetration via voice perturbation methods. Refine spatial delivery parameters to target specific body regions or neural correlates.

These tools support clinical practitioners in validating system performance, identifying underperforming modules, and optimizing protocol parameters per individual user characteristics.

### Application to Closed-Loop Neuromodulation

The synchronization framework described herein forms the basis of a closed-loop neuromodulation system that dynamically aligns sensory delivery with the user's neurological and physiological state. This ensures: Maximized neuromodulatory efficiency through phase-aligned stimulation. Personalized therapy protocols grounded in real-time neurophysiological data. Longitudinal reliability across sessions due to learned latency profiles and adaptive waveform correction.

Phase Alignment Algorithms. Using the latency data obtained, the system calculates the optimal phase alignment for delivering multi-sensory stimuli in a synchronized manner. These calculations are performed by the control software, which adjusts the timing, offset, and sequencing of each sensory channel so that: Signals are delivered with calibrated delays tailored to each user's neural processing profile. Neural responses to different modalities arrive in the brain concurrently or in controlled phase relationships. Cross-modal coherence is improved, thereby enhancing entrainment efficiency and minimizing phase-related desynchronization.

The system may store user-specific latency profiles, enabling future sessions to initialize with pre-configured phase parameters, reducing the need for repeated calibration.

In an additional embodiment, the system includes a suite of advanced synchronization mechanisms designed to ensure precise temporal alignment across all delivered sensory stimuli—namely vibroacoustic, auditory, and visual modalities. The system addresses the inherent challenge of multi-modal neuromodulation by employing a redundant synchronization architecture capable of both initial alignment and ongoing timing correction during therapy sessions.

### Redundant Synchronization System

To maintain high-fidelity entrainment and phase coherence, the system integrates multiple redundant sensor mechanisms that measure and verify the timing of stimulus delivery across sensory channels. These include: Photocells, positioned to detect flashes, LED pulses, or ambient light shifts corresponding to the timing of visual stimuli. These sensors ensure that visual outputs are correctly timed relative to other modalities and help confirm that no delay has occurred in signal execution. Microphones, integrated within or adjacent to speaker components, which monitor the temporal characteristics of auditory outputs. These may include measurements of onset time, amplitude envelope, and waveform fidelity, allowing the system to verify audio timing and adjust for speaker delays or environmental damping effects. Accelerometers, embedded within transducer elements or placed on the mat structure, which detect the timing, amplitude, and phase of delivered vibrational stimuli. These readings enable the system to monitor whether vibratory events occur as programmed and to detect any lag or phase drift caused by material or mechanical interference.

This multi-sensor architecture allows the system to cross-validate timing data across modalities, thereby improving the robustness and reliability of synchronization.

### Latency and Phase Control

A central synchronization module processes input from these redundant sensors and manages a latency control and phase alignment system that performs the following functions: Precision alignment of sensory stimuli: Using the measured latencies, the module calculates corrected delivery timings for each modality. Adjustments are applied in real time to ensure that stimuli across all channels arrive at the user's sensory system in coordinated phase, enhancing the neural integration of input and improving entrainment outcomes. Prevention of timing drift: The redundant monitoring system continuously checks for drift in any channel and applies corrective timing offsets as needed. These corrections may involve delaying or advancing specific outputs, adjusting buffer settings, or temporarily resynchronizing all channels based on a master timing signal.

The result is a system capable of maintaining stable, dynamic, and high-resolution synchronization throughout therapy sessions, regardless of environmental factors, hardware variance, or user-specific sensory latency. This capability is essential for multi-modal neuromodulation, where even minor phase mismatches can degrade therapeutic effectiveness.

### Phase Alignment Algorithms

In accordance with various embodiments, the system employs phase alignment algorithms that utilize latency data to calculate and enforce optimal timing and sequencing of multi-sensory stimuli—specifically vibroacoustic, auditory, and visual channels—such that neural responses to these stimuli arrive at the brain in coordinated phase relationships, thereby maximizing entrainment efficiency and reducing inter-modal desynchronization.

### Calibration of Delivery Delays Based on User-Specific Latency Profiles

The system measures the user's individual sensory processing latencies across modalities (vibratory, auditory, visual) and computes calibrated delivery offsets for each sensory channel. The control software uses the latency data to: Calculate delay offsets and sequencing for each modality tailored to the user's neural processing profile; Synchronize the arrival of stimulus-induced responses at the cortical level by delivering each modality with the appropriate temporal lead/lag; Improve cross-modal coherence by aligning modalities in phase and reducing phase-related misalignment of stimulus and neural response. User-specific latency profiles are stored in memory. Subsequent sessions may begin with pre-configured phase parameters drawn from the stored profile, thus reducing the need for full recalibration in each session and accelerating startup of phase-aligned therapy.

### Redundant Synchronization Mechanisms for Multi-Modal Coherence

The system includes a suite of redundant synchronization sensors and modules to maintain precise temporal alignment across all sensory stimuli and to correct for timing drift during sessions. These include: Photocells configured to detect the exact timing of visual stimuli (e.g., LED flashes, screen flickers), thereby verifying visual output onset in relation to other channels; Microphones positioned adjacent to speakers or audio outputs to capture acoustic onset, amplitude envelope, and waveform fidelity—used to verify auditory timing and compensate for speaker delay or environmental damping; Accelerometers embedded within vibroacoustic transducers or the support surface to detect vibrational onset, amplitude, and phase characteristics, thereby verifying whether vibrational events occur as intended and identifying mechanical or material-induced delays.

These sensors produce time-stamped data streams that are cross-validated by the central synchronization module to detect latency mismatches, phase drift, or channel drift, thereby improving the robustness and reliability of multi-modal synchronization.

### Latency Control, Drift Compensation and Real-Time Adjustment

A central synchronization control unit processes input from the redundant synchronization sensors and executes latency control and phase alignment functions including: Precision alignment of sensory stimuli: The unit uses measured latency offsets to apply corrected delivery timing for each modality in real time. Stimuli are delivered with calculated offsets so that the user's neural processing system receives coordinated inputs in phase. Prevention of timing drift: The system continuously monitors timing performance across modalities. If drift or desynchronization is detected (due to mechanical wear, temperature change, load variation, or body-movement shifts), the synchronization unit dynamically adjusts buffer delays, advances or delays individual channels, or triggers full resynchronization based on a master timing signal. Dynamic adaptation: During extended or complex therapy sessions, the system adapts timing offsets and phase relationships in response to evolving user state (e.g., fatigue, habituation, posture change) to maintain high-resolution phase alignment and entrainment fidelity.

### Impact on Multi-Modal Neuromodulation

By enforcing tightly controlled temporal and phase alignment of vibroacoustic, auditory and visual stimuli, the system ensures coherent multi-sensory delivery that aligns with the user's internal neural oscillations. Even minor phase mismatches can degrade therapeutic effectiveness—thus the system's phase alignment capability is critical to: Enhancing neural entrainment by aligning stimulus timing with endogenous oscillatory phase windows; Reducing sensory interference or cross-modal desynchronization that could obscure or diminish neuromodulatory impact; Improving user comfort, perceived immersion, and consistency of outcomes across sessions and users.

### Real-Time Synchronization Adjustments

In accordance with various embodiments, the system includes functionality for real-time adjustment of sensory stimulus synchronization based on the user's continuously monitored neural and physiological responses. This feature enables adaptive phase alignment during active therapy sessions, ensuring that multi-modal sensory stimuli—including vibroacoustic, auditory, and visual inputs—remain phase-locked and personalized as the user's neurological and physiological state evolves over time.

### Adaptive Timing Adjustment System

The system continuously acquires data from embedded biometric sensors and high-density EEG monitoring systems, allowing it to detect shifts in neural transmission latency or somatosensory processing. When such shifts are identified—due to user fatigue, neuroplastic adaptation, attentional fluctuation, or environmental factors—the control software executes real-time timing corrections that include: Temporal Offset Correction: Adjusting the timing of one or more sensory modalities (e.g., delaying auditory stimuli to compensate for slowed tactile perception or neural conduction delay). Amplitude and Envelope Shaping: Modifying the output waveform or intensity of stimuli to maintain smooth perceptual transitions and minimize disruptive signal discontinuities. Spatial Reprioritization: Reallocating vibrational emphasis to different body zones in response to varying regional sensitivity or transducer coupling efficiency detected during the session.

These corrections are applied in real time with minimal perceptual disruption, preserving the integrity of phase-aligned multi-modal stimulation throughout the session duration.

### Personalized Sensory Input Control

The system further comprises user interface elements (e.g., touchscreen GUI or clinician control console) that allow the user or practitioner to adjust and limit the intensity, frequency, and spatial distribution of each stimulus modality. Customization profiles may be stored and applied to: Define safe and comfortable operating ranges for each sensory channel based on user preference, clinical recommendation, or diagnostic history. Enable or disable specific stimuli (e.g., visual input for light-sensitive users, or vibrational output for post-operative zones). Preconfigure entrainment protocols aligned with individual biomarker profiles (e.g., gamma frequency peaks or alpha-phase suppression targets).

These user-specific profiles are dynamically modulated during therapy in response to real-time biometric feedback, ensuring continuous personalization.

### Neuroadaptive Feedback and Closed-Loop Responsiveness

In a further embodiment, the system implements closed-loop neuromodulation protocols wherein real-time feedback from EEG and biometric sensors drives continuous stimulus optimization. This closed-loop operation enhances therapeutic precision and responsiveness through: Neuroadaptive Correction: Automatically adjusting the phase alignment and stimulus parameters based on detected shifts in brainwave phase, power, or coherence. Stimulus Optimization: Maintaining optimal cortical entrainment across targeted frequency bands (e.g., alpha, beta, gamma) by dynamically modulating cross-modal coherence. Cognitive State Attunement: Adjusting stimuli to reduce cognitive dissonance, prevent overstimulation, and maintain engagement in users with sensory processing variability or neural instability.

This adaptive synchronization feature is particularly advantageous in use cases involving long-duration sessions, dynamic sleep staging, emotion-based variability, or clinical populations with volatile neural baselines (e.g., neurodevelopmental or neurodegenerative disorders). It ensures continuous alignment between system output and user state, thereby maintaining therapeutic safety, comfort, and efficacy.

### Latency Profile Generation and Application

In another embodiment, the system includes functionality for the generation, storage, and application of individualized latency profiles, which are used to pre-configure phase alignment parameters for multi-modal sensory stimulation. These latency profiles encapsulate user-specific neural response characteristics, enabling rapid session initialization, improved stimulus synchronization, and enhanced personalization of therapeutic protocols.

### Latency Profile Generation

During an initial calibration session or at periodic intervals, the system performs a structured multi-modal latency assessment to measure the user's neural and physiological response latencies across different sensory modalities. This process comprises: Stimulus Delivery and Sensing: The system sequentially or simultaneously delivers controlled vibroacoustic, auditory, and visual stimuli while capturing corresponding response signals using high-density electroencephalography (HD EEG), accelerometers, microphones, and other physiological sensors (e.g., galvanic skin response or heart rate monitors). Latency Mapping: The control software analyzes the temporal offset between the stimulus onset and the recorded response. This includes measuring: Sensory-specific response delays (e.g., time-to-response for vibration vs. audio). Regional propagation differentials (e.g., latency variation between lower and upper body vibrational inputs). Inter-modal synchronization parameters (e.g., required offsets to phase-align auditory and vibrotactile stimuli).

This data is processed to generate a comprehensive user-specific latency profile, which includes: Baseline Transmission Delays: Per modality (vibration, sound, light). Intermodal Offset Parameters: Required to achieve phase-synchronized cross-modal stimuli. Regional Timing Maps: Showing propagation delays as a function of body location and transducer position.

### Profile Storage and Retrieval

Once constructed, the latency profile is stored either: Locally, on the session control interface or therapy system. Remotely, in a secure cloud-based profile repository for cross-device access and long-term user tracking.

Each profile is associated with a unique user identifier and may be version-controlled to support incremental refinements over time based on ongoing biometric data collection.

### Profile Application During Therapy

During subsequent therapy sessions, the system accesses the stored latency profile to automatically pre-configure synchronization settings. This includes: Adjusted stimulus delivery timing to ensure phase alignment across all sensory modalities. Automatic calibration of transducer outputs based on previously measured regional vibration propagation patterns. Application of dynamic modulation thresholds and offsets, allowing the system to maintain real-time alignment with the user's known physiological response characteristics.

These pre-configured settings allow therapy to begin without the need for a new calibration phase at each session, significantly improving system usability and operational efficiency.

### Adaptive Profile Updating

In further embodiments, the system supports profile refinement by continuously monitoring user responses across sessions. As more latency and biometric data is accumulated, the latency profile may be updated using machine learning or statistical smoothing techniques, enabling: Greater personalization and responsiveness to user-specific changes (e.g., due to aging, medication, or health status). Improved predictive accuracy, enhancing future phase alignment and session efficacy.

### Benefits of Latency Profiles

The use of individualized latency profiles provides several operational and therapeutic advantages: Reduced Setup Time: Eliminates redundant calibration, enabling rapid session initiation. Precision Neuromodulation: Facilitates highly synchronized multi-modal entrainment tailored to the user's sensory timing profile. Therapy Optimization: Improves comfort, efficacy, and adaptability across use cases and user populations. Scalability: Supports seamless session switching and multi-user environments by preserving unique user data across devices.

This functionality forms a cornerstone of the system's precision neuromodulation architecture, enabling advanced personalization, temporal coherence, and longitudinal tracking of therapeutic impact.

### Control System Architecture and Closed-Loop Feedback

In accordance with various embodiments, the system comprises a centralized control and feedback architecture configured to orchestrate multi-modal sensory stimulus delivery, monitor real-time physiological and neurological biomarkers, and dynamically adapt session parameters in closed-loop fashion. The architecture supports both individual and multi-user (group) therapy sessions, enabling high-fidelity synchronization, adaptive modulation, and responsive personalization of therapeutic protocols.

### Central Control Unit

At the core of the architecture is a Central Control Unit (CCU) which is operative to generate stimulus signals, manage temporal synchronization across modalities, route signals to individual transducers and sensory modules, and oversee both standalone and networked session configurations. The CCU coordinates multi-channel outputs and receives feedback from sensors, thereby facilitating flexible session control in either single-user or multi-user environments.

### Signal Processing Chain

The control system incorporates an advanced signal processing chain that includes, but is not limited to: An amplifier module, which dynamically modulates power to vibroacoustic transducers, facilitating controlled adjustment of vibrational amplitude and waveform shape. A latency compensation module, which accounts for propagation delays across hardware, wiring, wireless channels, and body-mat coupling, thereby ensuring temporal alignment of sensory outputs. A phase control module, which ensures temporal waveform alignment across all output channels (vibration, sound, light) to maintain coherent phase-locked delivery and prevent desynchronization.

### Multiple Transducer Channel Outputs

The system is configured to simultaneously drive multiple distinct transducer outputs (channels 1 through N), each individually addressable in terms of frequency, amplitude, timing, and phase. This multi-channel architecture enables customization of stimulation protocols to target specific anatomical zones, accommodate varying user morphologies, or distribute stimuli across multiple users in parallel.

### Group Therapy Integration

The architecture further supports a group-therapy mode in which multiple users or multiple devices are orchestrated synchronously. The CCU administers low-latency synchronization protocols enabling real-time coherence of stimuli across users. Feedback from multiple participants may be collected and aggregated, allowing dynamic adjustment of shared session parameters and synchronized multi-user entrainment experiences.

### Feedback Systems

To facilitate closed-loop control, the system integrates a suite of sensors and feedback mechanisms, including: Accelerometers positioned at specific body-locations or embedded within mats or transducer assemblies to monitor vibrational output, coupling fidelity, and physical response. Photonic sensors (photo cells) configured to detect visual stimulus onset and timing (e.g., LED pulse emission) to ensure alignment of visual modality with other channels. Microphones located in proximity to audio outputs capturing acoustic emission timing, waveform quality, and external perturbations such as vocal resonance. High-density EEG subsystems, which monitor brain-wave activity in real time, providing neural biomarker data used to validate entrainment, adjust stimuli, and assess therapeutic effect.

### Machine State and Biomarker Tracking

The CCU continuously monitors both machine-state variables (e.g., module activity, error or fault states, transducer temperature, connectivity status) and user biomarker variables (e.g., EEG frequency band power, heart rate variability (HRV), galvanic skin response (GSR), respiration rate). These inputs are processed to guide real-time session adjustments and ongoing protocol refinement.

### Closed-Loop Feedback Pathways

A central feature of the architecture is a closed-loop feedback pathway enabling: Real-time monitoring of system outputs and user biometrics. Continuous adaptation of stimulus parameters based on biometric responses or neural state changes. Precise synchronization of multi-modal inputs across time and spatial dimensions to maintain optimal entrainment conditions.

### Key Capabilities Enabled by the Architecture

The control and feedback architecture enables: Real-time monitoring and adjustment, ensuring that vibrational, auditory and visual stimuli remain aligned with the user's current physiological and neurological state. Multi-modal feedback integration, coordinating vibration, sound, light, and EEG data streams to deliver a coherent therapeutic experience. Synchronized group therapy capabilities, supporting shared neuromodulation sessions with multiple users or remote locations, synchronized via centralized control. Advanced biomarker tracking, enabling detailed insight into physiological and cognitive changes over time, supporting long-term monitoring and personalization. Dynamic therapy optimization, continuously refining stimulus parameters, session sequencing, and modality integration based on real-time user feedback and performance metrics.

The control system architecture described herein provides a flexible, scalable, and responsive platform for precision neuromodulation across individual and collective contexts, ensuring synchronized, adaptive, and outcome-driven therapeutic stimulation.

### Neural Entrainment and Brain Data Processing

In accordance with various embodiments, the system comprises a neuromodulation architecture that integrates vibroacoustic stimulation with high-density electroencephalography (HD EEG) monitoring and real-time biometric feedback to generate personalized entrainment protocols. The system captures, analyzes, and applies neural signals to dynamically adapt sensory stimulus delivery across vibration, audio, and visual modalities, thereby enhancing therapeutic effect.

Vibroacoustic Setup with HD EEG Integration

The therapy surface comprises a modular topper layer designed to interface with transducer assemblies and physiological sensors. Under-body vibroacoustic transducer elements deliver upward directed vibrational energy into the user's body, while over-body transducer elements provide a cocooning vibrational field enveloping the user in synchronized energy. A physiological sensor affixed to the user continuously monitors biomarkers such as heart rate and galvanic skin conductance, the data of which feed back into the session control loop in real time. Localized transducer pucks may be positioned on specific body regions (hands, feet, torso) to deliver focused vibroacoustic stimuli aligned with neural or biometric patterns. A light-shield is employed to reduce ambient visual interference and enhance immersive effect. Auditory stimuli are delivered through headphones synchronized with vibroacoustic and visual signals to create a coherent multisensory experience. HD EEG sensors mounted on the user's scalp acquire neural activity across gamma, alpha, beta, theta and other oscillatory bands; this data is utilized in real-time neurofeedback and entrainment targeting. Optionally, an AR/VR display module with eye-tracking may be included to present synchronized visual stimuli and capture gaze dynamics for refined engagement.

### Brain Data Recording and Processing Flow

Resting-State Brain Data Recording. The system collects baseline neural data while the user is in a resting state. Spectral analysis (e.g., fast Fourier transform) is applied to identify baseline dominant frequency bands, harmonics and noise characteristics. This resting-state data serves as a reference for further modeling.

Task-Dependent Brain Data Recording. During controlled tasks or exposure to stimuli, additional EEG data is acquired. These task-state data are analyzed to reveal how the user's neural dynamics change during active stimulation or cognitive load.

Perceptual-Based Individual Gamma Estimation. The system estimates the user's optimal gamma frequency by analyzing psychophysical responses near threshold levels. This process may involve modulating signal or noise combinations within the gamma band and adjusting until resonance-based peaks emerge. These estimates complement the HD EEG neuromap data and refine frequency-targeting.

### Data Processing and Mapping

Spectral Data Processing. Both resting-state and task-state EEG signals undergo spectral transformations to extract power distributions across frequency bands. Dominant peaks, harmonic relationships and power distributions inform the development of further models.

Personalized Brain Network Map. Using spectral and spatial data, the system constructs a brain-network connectivity map showing how different brain regions communicate and oscillate in concert. This map guides entrainment targeting and stimulus placement.

Identification of Individual Gamma Frequency. Based on spectral peaks and differential responses, the system determines the user's prime gamma frequency (e.g., 43 Hz rather than a generic 40 Hz). This individualized frequency is then used to tailor stimulus generation.

Spatio-Temporal Latency and Phase Mapping. The system calculates temporal latencies and spatial phase differences in the user's neural responses, enabling precise alignment of sensory inputs so that stimuli arrive in phase-synchronous relation to endogenous neural activity.

### Optimized Sensory Neural Entrainment Model

Model Development. Drawing on individual brain network maps, identified gamma/other frequency targets, and latency/phase mapping data, the system generates an optimized entrainment model. This model defines how, when and where the vibroacoustic, auditory and visual stimuli should be delivered to maximize phase alignment and neural resonance.

Single or Multi-Modal Delivery. Depending on the user's profile and therapeutic goals, the system supports delivery in single modality (e.g., vibroacoustic only) or multi-modal synchronization (e.g., vibration+sound+visual flicker). The protocol is selected to best engage the user's unique brain dynamics, with stimuli timed and phased for maximal alignment and entrainment effect.

### Calibration and Feedback Systems

In accordance with various embodiments, the system includes a dynamic calibration and feedback subsystem designed to ensure accurate, consistent, and personalized energy delivery across all vibroacoustic transducers and sensory outputs. Calibration is critical to maintaining therapeutic fidelity, particularly when user positioning, body composition, or environmental acoustics are variable. To accommodate different use scenarios and practitioner preferences, the system supports multiple calibration methodologies and levels of modeling fidelity.

### Calibration and Monitoring Device

The system provides a handheld calibration device comprising a spectrogram interface and at least one sensor (e.g., microphone, and optionally an accelerometer). This device is configured to log amplitude, frequency, and harmonic content at discrete spatial locations around the user's body—both above and below the support surface. It enables detection of unwanted resonance, amplitude drift, spatial coupling anomalies, and vibrational penetration depth via voice-perturbation protocols (wherein the user hums during vibration and the resulting deviations in the spectrogram are analyzed). Because the calibration device is portable, it may be repositioned to different anatomical zones (e.g., abdomen, limbs, back) to support localized adjustment of transducer gains, phase offsets, or spatial coupling to compensate for anatomical variances.

### Vibroacoustic Platform Setup

The calibration subsystem works in concert with the therapy platform, which comprises: A structural base platform serving as the support and positioning surface for the user. A modular topper module which is interchangeably selected (e.g., foam overlay, gel pad, pneumatic air-cell topper) to suit differing therapy profiles. Modular transducer elements are arranged both below and above the user's body to provide a 3-D cocooning vibrational field, enabling immersive stimulation and selective intensification or attenuation of specific anatomical zones. A physiological sensor monitors biometric signals (such as heart rate or skin conductance) to inform closed-loop vibrational adjustments. Localized transducer pucks may be placed at anatomically critical locations (e.g., hands, feet, torso) to deliver high-intensity focused vibration in alignment with protocol targets. Optional components include a light-shield to reduce ambient visual interference, and headphones to deliver synchronized auditory stimuli aligned with vibrational and visual cues. A control interface and visualization/display module allow the practitioner or user to monitor session metrics and adjust parameters such as amplitude, frequency, and phase settings in real time.

### Simplified Calibration and Logging Model

In embodiments oriented toward ease and practicality, the calibration subsystem operates in a simplified mode that records key parameters—specifically amplitude and frequency at selected calibration points. The user or practitioner manually fine-tunes transducer settings (for example, adjusting gain or offset) based on the logged data. This simplified mode is particularly suitable in settings with limited computational or modeling resources or when rapid deployment is required without full system modeling.

### Complex Model for Comprehensive Data Integration

For advanced therapeutic or research contexts, the system supports a comprehensive calibration model that integrates multiple physiological, environmental, neural, and structural data streams. Features of the complex model include: Volumetric Energy Distribution Mapping: Spatial modeling of how vibrational energy propagates through the user's body, identifying zones of under- or over-stimulation. Volumetric Biophysics Functional Brain Data: Integration of neural activity (e.g., from HD EEG) with vibrational inputs to monitor neurophysiological responses in three dimensions. Cognitive and Perceptual Change Monitoring: Tracking the evolution of the user's mental or perceptual state during or after sessions, providing feedback for personalization. Circulation Effects: Measuring changes in blood flow or microcirculation induced by vibrational therapy, thereby providing physiological validation of therapy effectiveness and informing intensity modulation. Body/Head Physical Modeling: Incorporation of anthropometric and biomechanical properties (such as tissue density, bone geometry, body mass distribution) to predict vibrational absorption, reflection, and coupling characteristics. Latency/Modality Interaction Tracking: Monitoring temporal delays, phase interactions and modality interplay (vibration, audio, visual) to fine-tune synchronization. Phase-Amplitude Coupling Analysis: Assessing relationships between vibrational phase, amplitude and neural entrainment thresholds to optimize stimulus configuration. Percent Brain Entrainment Achieved: Computing, based on neuromap targets, the proportion of brain oscillatory activity successfully entrained during a session.

By supporting both simplified and complex calibration models, the system remains flexible: operable in lighter, manually adjusted modes or fully data-driven adaptive modes. The complex model empowers precision control, enabling continual refinement of therapy sessions through feedback, modeling and personalization, thereby making the vibroacoustic platform responsive to individual neural and physiological states.

### Manual Calibration Modes

In one embodiment, the vibroacoustic therapy system includes a manual calibration mode, enabling trained practitioners to perform real-time, user-specific adjustments to system parameters based on observational assessment, sensor feedback, and patient response. This calibration method is intended for clinical and therapeutic environments where individualized attention to anatomical, physiological, or therapeutic nuances is required.

Manual calibration procedures may include, but are not limited to, the following adjustments: Transducer Gain Tuning: Practitioners may increase or decrease the power output of specific transducer elements to optimize vibrational intensity across targeted body zones. Gain adjustments may be performed in response to observed under-stimulation or discomfort and are particularly relevant in regions with variable tissue density or contact pressure. Spatial Focus Adjustment: The practitioner may manually reposition transducer pucks, perimeter modules, or over-body elements to redirect vibrational energy toward specific anatomical structures, such as the vagus nerve, spinal column, or muscular trigger points. This allows for anatomical targeting that aligns with clinical objectives or user-reported sensations. Waveform Parameter Modification: Practitioners may alter waveform characteristics, such as frequency, amplitude envelope, or duty cycle, to shape the perceived quality of vibration. For example, sinusoidal waveforms may be replaced with square or triangle waves to modulate the depth of penetration, pulsation feel, or harmonic richness of the stimulus. Multi-Zonal Balance Tuning: In systems with multiple independent transducer zones (e.g., torso, legs, arms, head), manual calibration may involve balancing output levels across zones to ensure symmetrical or asymmetrical distribution patterns appropriate to the therapy plan.

Sensor feedback from accelerometers, microphones, or physiological monitors may be used in conjunction with practitioner intuition and user feedback to guide calibration decisions. For example, real-time accelerometer data may confirm vibrational amplitude at the skin surface, while galvanic skin response (GSR) or facial expression monitoring may reflect emotional or stress-related reactions to the stimulus.

This manual calibration capability is especially beneficial in the following contexts: Initial System Setup for New Users: Where no latency profile or historical data exists, manual tuning provides a fast and intuitive method of achieving comfortable and effective stimulation. Therapeutic Sessions Requiring Custom Attention: Including cases involving trauma-informed care, pain sensitivity, or anatomical anomalies. Rapid Intervention During Therapy: Enabling practitioners to respond dynamically to user feedback or biometric signals that indicate overstimulation, discomfort, or desynchronization.

Manual calibration complements automated and closed-loop modes by offering a flexible, practitioner-driven mechanism for ensuring optimal stimulus delivery. It supports clinical personalization without requiring complex modeling or algorithmic control and forms a foundational capability within the broader calibration subsystem of the system.

### AI Driven Calibration Modes

In accordance with various embodiments, the system employs an AI-driven calibration mode, wherein real-time sensor data and machine-learning algorithms are utilized to autonomously optimize energy distribution, stimulus timing, amplitude, waveform shape, and phase alignment across the vibroacoustic, auditory, and visual subsystems. This AI module continuously analyzes feedback from devices such as accelerometers, microphones, and other sensing modalities, with the objective of improving energy coupling, reducing signal loss, maintaining phase coherence, and enhancing user-specific therapeutic efficacy.

Goal-Oriented Session Customization Framework. The calibration and session configuration process is integrated with a goal-oriented framework that begins with an interview process (conducted by AI, by a human operator, or a hybrid thereof) to determine the user's current needs, preferences, and therapeutic objectives (e.g., stress reduction, focus enhancement, physical rehabilitation, sleep optimization). The selected goal informs module and mode selection, which can proceed automatically via AI (based on pattern recognition and prior session data), manually via practitioner control, or through a hybrid approach combining AI suggestions and expert oversight. The selected mode(s) may include, but are not limited to: Massage Mode, Body Mapping Mode, Sound Therapy Mode, Rehabilitation Mode, Sensory Deprivation Mode, Entertainment Mode, Social Mode, Mobile Mode, Entrainment Optimization Mode, or combinations thereof.

Autonomous Calibration and Adaptation. During the calibration phase and the ensuing therapy session, the AI calibration module: Continuously ingests sensor feedback from microphones (acoustic signal onset and integrity), accelerometers (vibrational amplitude, phase and waveform fidelity), physiological sensors (e.g., heart rate variability, galvanic skin response), and optionally EEG/neuromap data. Evaluates whether vibrational amplitude, waveform shape, spatial distribution, timing offsets, and modal coherence align with the therapeutic target profile defined in view of the user's neuromap, physical coupling, and prior session history. Automatically adjusts system parameters including transducer gain, waveform shape (e.g., sinusoid vs. square vs. pulse train), frequency, amplitude envelope, temporal offsets, spatial zoning of output, and phase alignment across modalities to optimize energy transfer, reduce attenuation, and maintain phase-locked stimulation across vibration, sound and light channels.

Post-Session Feedback and Model Refinement. At the conclusion of each session, the system performs a post-session assessment involving user feedback, biometric sensor data, and system performance logs. The AI module logs this data, updates the user's calibration profile, and refines the machine learning model to improve future performance and personalization of subsequent sessions. Over time, the AI calibration subsystem evolves the user-specific stimulation protocol tree, reducing the need for manual adjustments and enabling a progressively refined therapy path tailored to the user's evolving neural, physiological and preference-based profile.

Hybrid Control and Practitioner Oversight. While the AI subsystem may operate autonomously, the architecture supports practitioner or user override, hybrid manual/AI mode selection, and manual fine-tuning when required. The interface facilitates review of AI-generated suggestions, module and mode selection, session parameter preview, and manual intervention. This hybrid control capability ensures that the AI calibration process aligns with practitioner judgement, user comfort, and safety considerations.

Benefits and Advantages. The AI-driven calibration mode offers several key advantages: Reduced calibration time and user burden compared to purely manual tuning; Enhanced precision in energy delivery and phase alignment, improving neuromodulatory effect; Improved adaptability to changes in user anatomy, coupling conditions, session context or environment; Ability to continuously learn and improve across sessions, supporting longitudinal personalization; Scalability for multi-user and group therapy settings via standardized AI models and individual calibration profiles.

### Hybrid Calibration and Sensor Feedback Loops

In accordance with various embodiments, the system supports a hybrid calibration mode combining practitioner manual input with automated machine-learning adjustments. This hybrid mode provides the flexibility for trained users or clinicians to override or refine AI-generated recommendations, while benefiting from real-time optimization based on sensor feedback and predictive analytics.

### Calibration and Monitoring Device

The system includes a portable calibration and monitoring device featuring a spectrogram interface and integrated sensors (for example, a microphone and accelerometer). The device is moveable around the user (above, beside, or beneath) to capture localized vibration amplitude, frequency, harmonic content, spatial distribution, and depth of energy penetration. In certain embodiments, the device supports a voice-perturbation protocol wherein the user hums in the presence and absence of vibration; the difference in spectral signatures is computed to infer vibrational coupling to internal tissues and effective energy penetration. This tool enables the practitioner to fine-tune session parameters—such as transducer gain, phase offset, or spatial coupling—in accordance with anatomical or physiological characteristics of the individual user.

### Vibroacoustic Platform Setup

The therapy surface comprises a main platform or therapeutic table on which the user is positioned. A modular topper module is placed above the base and may be replaced with alternative layers (such as comfort foams, gel overlays, or air-cell modules) to support varying therapy configurations. Modular vibroacoustic transducer elements are mounted both below and above the user's body, enabling the creation of a three-dimensional (3-D) vibrational field. This dual placement permits: immersive full-body vibrational stimulation enveloping the user in a coherent tactile field; directed and localized energy adjustments to specific anatomical zones (for example, extremities, core, lateral surfaces).

Physiological sensors (e.g., heart rate monitors or galvanic skin response sensors) monitor the user's biometric state in real time during therapy; data from these sensors feed into the session control logic to adapt parameters for safety and efficacy. Localised transducer pucks may be positioned adjacent to hands, feet, or torso to deliver increased stimulation intensity in targeted regions. An optional light-shield may be deployed to block external visual stimuli, thereby fostering sensory isolation and enhanced internal focus. Headphones deliver auditory stimuli synchronized with the vibrational pattern. A practitioner or user-accessible control interface and visualization module enable monitoring and manual adjustment of key parameters—such as vibrational frequency, amplitude, phase offsets, and session timing—thus providing full control over the therapeutic experience.

### Feedback Devices and Diagnostic Tools

In accordance with various embodiments, the system incorporates a suite of feedback devices and diagnostic tools designed to support both manual and AI-driven calibration modes. These tools enable the sensing, visualization, and quantitative analysis of stimulus effectiveness across vibroacoustic, auditory, and visual modalities, thereby ensuring precise, repeatable, and context-aware energy delivery for each user.

### Handheld Calibration and Feedback Device

The system may include a handheld calibration device, configured with a spectrogram display, an integrated microphone, and an accelerometer for multi-dimensional feedback capture. This device is operable to: Measure vibrational amplitude, frequency distribution, waveform integrity, and harmonic content at discrete body regions. Visualize frequency spectra in real time, enabling practitioners to identify resonant peaks, energy drop-offs, phase cancellations, and non-uniform propagation zones. Assess vibrational penetration depth through comparative analysis of measured amplitude decay, providing direct insight into energy transfer efficiency.

The spectrogram interface presents an instantaneous visual representation of the vibrational signal across time and frequency domains, facilitating immediate diagnostic interpretation. Practitioners may reposition the handheld device above, below, or lateral to the user's body to sample energy delivery across multiple anatomical planes, ensuring uniform coverage and confirming correct transducer coupling.

### Voice Perturbation Analysis Protocol

In certain embodiments, the system further employs voice perturbation analysis as a non-invasive diagnostic mechanism for evaluating internal vibrational coupling. During this procedure, the user produces a sustained vocalization (for example, humming or vowel tones) under both vibrational and non-vibrational conditions. The handheld calibration device records the acoustic waveform, and embedded algorithms compute spectral differences between conditions to determine: The degree of internal resonance shift caused by vibration. The vibrational energy transmission depth within soft tissue or cranial cavities. Phase and amplitude modulations in the user's natural resonance profile, which serve as indirect markers of therapeutic energy penetration and field coherence.

This methodology provides a sensitive diagnostic approach for validating transducer placement, system synchronization, and vibrational effectiveness without requiring invasive instrumentation.

### Integrated Feedback Network

The feedback and diagnostic tools described herein interface directly with the system's central control unit or AI-driven calibration engine, feeding continuous sensor data (amplitude, phase, latency, frequency response) into closed-loop algorithms. The resulting data streams are analyzed in real time to dynamically adjust transducer gain, waveform shape, phase offset, and timing synchronization. Practitioners may override or supplement AI adjustments through the system's manual calibration interface, ensuring clinical oversight while maintaining automated optimization.

### Enhancing Therapy Precision and Consistency

The integration of these feedback mechanisms enables precise fine-tuning of energy delivery and ensures that vibrational stimuli remain therapeutically effective across users and sessions. Specifically, the system ensures that vibrational output: Reaches designated anatomical or neurological targets with controlled amplitude and spectral purity. Maintains waveform coherence despite variations in user posture, tissue density, or environmental conditions. Adjusts dynamically to account for transient physiological changes such as heart rate variability, respiration rate, or galvanic skin response fluctuations.

Through this combined manual-AI calibration architecture, the system delivers consistent therapeutic outcomes across diverse clinical, research, and wellness environments while safeguarding user comfort and safety.

### Software and AI-Driven Control

The vibroacoustic therapy system further comprises a software platform accessible through a tablet, smartphone, or desktop interface, which manages, customizes, and automates therapy sessions. The software integrates AI-driven control algorithms for personalization, adaptive optimization, and continuous learning based on sensor and feedback data.

### Control Interface and User Profiles

The control interface allows: Selection of pre-programmed or custom therapeutic protocols. Adjustment of transducer parameters such as intensity, waveform, frequency, duration, and timing. Synchronization of vibroacoustic, auditory, and visual stimuli. Configuration of session categories (e.g., relaxation, focus, neurorehabilitation, or sleep).

Each user profile stores: Preferred configuration and sensitivity thresholds. Historical EEG, biometric, and calibration data. Frequency targets and entrainment response maps. Profiles may be stored locally or in an encrypted cloud repository, enabling cross-device access and longitudinal personalization.

### AI-Powered Session Adaptation

Machine-learning models trained on latency maps, historical session outcomes, and real-time sensor feedback dynamically modulate therapy parameters. The AI engine: Adapts vibrational frequency to match evolving EEG patterns or physiological states. Adjusts spatial energy distribution in response to user posture, stress, or biometric indicators. Varies session pacing or duration based on user state transitions (e.g., shifting from gamma stimulation to slow-wave entrainment during sleep).

### Real-Time Monitoring and Feedback Visualization

During sessions, the software continuously aggregates inputs from EEG interfaces, calibration sensors, and environmental monitors. Practitioners may view: Live EEG spectrograms and neural coherence maps. Vibrational amplitude heatmaps and zone diagnostics. System alignment indicators and real-time biomarker dashboards. Automated alerts and corrections are triggered if sensor deviations, signal drift, or abnormal physiological readings are detected.

### Protocol Development and Customization Tools

The platform supports custom protocol creation through a graphical interface, allowing clinicians to: Define waveform and frequency timelines. Implement conditional logic (“if EEG alpha >12 Hz, reduce vibration intensity”). Integrate external biofeedback or neurofeedback systems.

These tools support both individualized and scalable deployment across institutional or research environments.

### Cloud Connectivity and Group Synchronization

In multi-user or group therapy modes, the software enables synchronized operation of multiple systems using low-latency wireless protocols. The AI engine can align individual sessions to collective EEG phase trends, facilitating shared entrainment experiences and population-level biomarker analytics. Data integrity and privacy are maintained through encryption, anonymization, and user-controlled consent.

### Goal-Oriented Customization and Mode Selection

In accordance with various embodiments, the system comprises a goal-oriented customization framework configured to dynamically configure therapeutic or sensory sessions in response to user objectives, preferences, and clinical endpoints. The architecture supports flexible mode selection—from basic massage protocols through fully integrated multi-sensory neuromodulation sequences—using an intelligent interview/assessment process driven by artificial intelligence (AI), human practitioner logic, or a hybrid combination.

### Customization Workflow and Mode Selection

Interview/Assessment Phase. At the outset of a session, the system prompts the user (or practitioner on behalf of the user) with an interview or assessment module designed to elucidate the user's current objectives (e.g., relaxation, cognitive enhancement, pain relief, sleep quality), historical sensitivity or sensory preferences, prior therapy responses, and physiological constraints.

The assessment module may operate in one of three fashions: AI-Decision Logic: The system algorithmically analyzes user input, biometric/neuromap history, and other datasets to suggest optimal stimulation modalities, appropriate frequency bands, and protocol sequencing. Expert Ruleset: A practitioner-driven logic set may allow manual or guided mode selection in contexts requiring clinical judgement. Hybrid Approach: The system generates AI recommendations which are reviewed and refined by a human operator, combining computational insight and practitioner oversight.

Session Goal Identification. Based on the assessment output, the system establishes a session goal which guides the configuration of therapeutic modules. Possible goals include, but are not limited to: stress reduction, muscle tension relief, cognitive enhancement, sleep induction, rehabilitation, pain modulation, or neural entrainment mapping.

Module and Mode Selection. The system selects and configures one or more sensory modules (e.g., vibroacoustic transducers, auditory and visual entrainment devices, TES/TMS modules, sensors for body-mapping) and spatial distributions aligned with the session goal.

In AI-mode, the system autonomously selects the optimal combination of modules, spatial placements, and stimulation parameters based on prior data and neuromap profiles.

In Manual-mode, a human practitioner directly selects modules, adjusts intensity or placement, or modifies the stimulation modality explicitly.

In Hybrid-mode, the system presents AI-based module suggestions and the practitioner refines or overrides as needed.

During this stage, the system configures key parameters such as transducer placement and intensity, timing, latency and phase offsets across modalities, auditory and visual stimulus profiles, and safety/comfort thresholds based on the user's prior sensitivity data.

Post-Session Assessment and Refinement. Upon completion of the session, the system engages a post-session assessment workflow, wherein user feedback (e.g., subjective comfort ratings, responsiveness) and objective sensor data (EEG changes, biometrics, calibration logs) are collected and analyzed. The system generates recommendations for subsequent sessions, updates the user's profile, and refines default parameters or mode selection logic. Over time, this iterative loop of assessment, adaptation and personalization enhances the accuracy, comfort and effectiveness of each therapy session.

### Mode Selection Options

In one embodiment, the system's interface presents a mode-selection panel populated with both preset and customizable modes, each tailored to specific therapeutic, immersive or entertainment objectives. Available modes may include (but are not limited to):
Massage Mode: Vibroacoustic and tactile stimulation focused on muscle relaxation, tension reduction and stress mitigation.
Body Mapping Mode: Integrated sensors capture real-time physiological responses (e.g., skin conductivity, vibrational resonance, muscular tension), enabling dynamic adjustment of stimuli for precision targeting and individualized therapy.
Sound Therapy Mode: Acoustic stimuli tuned to the user's personalized neuromap, driving brainwave entrainment via auditory pathways to promote relaxation, focus or cognitive modulation.
Rehabilitation Mode: Aimed at physical or neurological recovery, combining vibration, sound, biofeedback, and optionally TES or TMS to support motor retraining, sensory reintegration or neuroplastic adaptation.
Sensory Deprivation Mode: Reduces extraneous sensory input (e.g., dimmed lighting, acoustic isolation, visual occlusion) to foster introspective states, deep meditation or enhanced internal focus while applying controlled stimulation.
Entertainment Mode (Game/Theater/Music): Configures the system as an immersive multimedia platform wherein vibration, audio and visual stimuli are synchronized to enrich entertainment or artistic experiences.
Social Mode: Configures multiple user systems for synchronized group therapy or co-experiences, aligning sensory inputs across participants for shared entrainment or collective modulation.
Mobile Mode: Adapts the system for portable or wearable configurations supporting vibroacoustic or sensory modules during travel, commuting, or outdoor settings.
Entrainment Optimization Mode: Leverages the user's neuromap to dynamically tailor sensory stimuli (for vibration, sound, visual flicker) aimed at enhancing neural entrainment in targeted frequency bands (e.g., gamma, alpha, theta).
Combination Mode: Allows layering of two or more modes (for example, Massage+Entrainment or Rehabilitation+Entertainment), enabling complex, multi-dimensional experiences aligned with mixed therapeutic objectives.

The interface thus provides a versatile and adaptive control panel enabling rapid reconfiguration of the system's sensory, physiological, and neuromodulatory parameters in accordance with the user's stated goal, session context, biometric state, and neuromap profile.

### Calibration and Redundant Synchronization

Calibration of the foldable and modular vibroacoustic platform is essential to ensuring consistent, high-fidelity delivery of vibrational, auditory, and visual stimuli across the system. To this end, the system incorporates multiple layers of real-time monitoring and feedback for synchronization across sensory modalities and structural segments.

Key calibration components include:
Photocell sensors: These sensors detect actual light emissions—such as screen flicker or LED flashes—to validate the precise timing of visual stimuli, thereby ensuring that visual entrainment protocols are accurately synchronized with both auditory and vibroacoustic inputs.
Accelerometers: Embedded within or attached to each fold-panel segment or at each transducer location, the accelerometers measure vibrational amplitude, phase and onset timing. This enables detection of frequency drift, latency mismatches, or improper transducer function, and supports real-time phase correction or amplitude adjustment across the mat.
Microphones: Integrated microphones serve a dual purpose: capturing external audio cues and measuring voice-perturbation responses. This allows fine-tuning of interference effects, verification of combined audio-vibrational output and real-time adjustment of system output relative to expected audio-vibrational patterns.

Together, these redundant calibration mechanisms form a robust feedback loop that ensures each mat segment, each transducer puck, and each raised-perimeter module operates in phase and at the intended frequency. Such calibration enhances therapeutic efficacy by preserving phase coherence and by minimizing latency mismatches across the system—characteristics particularly critical when the system is used for high-resolution neuromodulation or brainwave-entrainment protocols.

### Portable and Wearable Configurations

Adaptability and Accessibility. In an additional embodiment, the disclosed system provides for portable and wearable configurations of the vibroacoustic therapy system, thereby extending its therapeutic capabilities beyond traditional stationary or clinical environments. These configurations are engineered to support increased adaptability, mobility, and user accessibility, enabling continuous or intermittent therapeutic engagement in non-traditional settings such as during travel, daily activity, seated work, or dynamic physical movement.

The portable and wearable embodiments maintain core functionality—including vibrational energy delivery, synchronized multi-sensory stimulation, biometric monitoring, and adaptive calibration—while being miniaturized, modularized, or reconfigured for compact integration into mobile or body-worn form factors.

Such adaptations are designed to facilitate broader therapeutic use cases across diverse user populations and environments, while preserving the precision, synchronization, and closed-loop feedback systems characteristic of the full-scale therapy platform.

### Handheld Modules

In one embodiment, the system comprises handheld vibroacoustic modules configured to deliver targeted vibrational stimulation in portable or non-clinical environments. These modules incorporate one or more embedded transducer elements, sensor arrays, and user interface components within a compact housing, enabling flexible, user-directed application across various anatomical regions.

Each handheld module may include:
A vibroacoustic transducer for localized vibrational output;
An onboard accelerometer or microphone for real-time monitoring of output amplitude and waveform fidelity;
A control interface—such as a touch-sensitive surface, physical button, or wireless interface—for adjusting intensity, frequency, and operational mode;
A wireless communication module (e.g., Bluetooth, Wi-Fi) for pairing with a centralized control platform or cloud-connected user profile;
A power source, such as a rechargeable battery, to enable autonomous operation independent of a base platform.

These portable devices may operate in standalone mode, delivering preprogrammed or user-selected vibration protocols, or in synchronized mode, where they function as an extension of the primary vibroacoustic system. In the latter case, the handheld unit receives timing and modulation data from the central control system, ensuring phase coherence and entrainment alignment with other sensory modalities.

The handheld module is designed for use in mobile environments including but not limited to: office settings, vehicles, aircraft, public spaces, or while lying or seated. This embodiment supports continuous or on-demand therapeutic access, enhancing the practicality and accessibility of vibroacoustic intervention across diverse use cases.

### Wearable Transducers and Accessories

In another embodiment, the system comprises wearable vibroacoustic modules configured for integration into garments, accessories, or support structures such as cushions or wraps. These wearable devices are engineered to deliver localized vibrational therapy while maintaining ergonomic conformity and user mobility across a range of physical states, including seated, ambulatory, or reclining positions.

Each wearable module may include:
One or more miniaturized vibroacoustic transducers embedded within flexible substrates or textile layers;
Integrated physiological sensors (e.g., heart rate, GSR, or accelerometry) to enable real-time biofeedback and adaptive stimulus modulation;
A wireless interface for communication with a centralized control platform or user device, supporting synchronization with other system modules and cloud-based user profiles;
An embedded power supply (e.g., lithium-polymer battery) for independent operation, with optional inductive or USB-C charging;
Optional attachment mechanisms (e.g., Velcro®, elastic bands, magnetic clasps) to maintain precise positioning against anatomical targets such as the thoracic spine, lumbar region, upper trapezius, or wrists.

Wearable configurations may operate in one of two modes:
Autonomous mode, wherein the device executes pre-configured stimulation protocols based on user-defined schedules or biometric thresholds; or
Synchronized mode, wherein wearable units function in phase-locked coordination with a central vibroacoustic platform, auditory stimuli, or EEG-informed entrainment protocols.

These wearable transducer systems facilitate discreet, continuous, or on-demand neuromodulation in everyday environments, extending therapeutic access and promoting long-term adherence through passive or low-profile integration into the user's lifestyle.

### Use in Mobile or Daily Life Scenarios

In yet another embodiment, the system supports a range of portable and wearable configurations engineered for use in mobile, everyday, or non-clinical environments. These configurations are designed to extend the therapeutic utility of the vibroacoustic system beyond stationary or practitioner-supervised deployments, thereby facilitating lifestyle-integrated neuromodulation and expanding user access.

Representative embodiments include, but are not limited to:
A handheld smart device, comprising one or more integrated vibroacoustic transducers, an accelerometer, a microphone, and optionally a spectrogram display. This device is operable as both a therapeutic actuator and diagnostic tool, allowing users to monitor vibrational amplitude, frequency distribution, and energy penetration in real time across different body regions.
A meditation cushion, internally equipped with transducer modules and configured for ergonomic use during seated postures. This unit delivers vibrational stimuli focused on the pelvic floor, sacral region, and lower spine, and may operate either independently or in synchronization with external auditory or visual entrainment modules.
Wearable transducer modules, embedded in garments, straps, wristbands, or attachable accessories. These units are miniaturized for discrete application and may be programmed for continuous, scheduled, or event-triggered operation. Control may be effected via manual interface, mobile application, or cloud-based session scheduler. These modules are optimized for use during meditation, walking, reclining, or ambulatory routines that benefit from mobile stimulus delivery.

These mobile and wearable extensions allow for therapeutic continuity across various use cases and physical environments, providing users with:
Uninterrupted access to neuromodulation protocols during daily life, including settings such as workspaces, homes, or transit vehicles;
Reduced dependency on full-body mat deployment, supporting low-footprint and flexible implementation;
Therapeutic inclusion for users with limited mobility or logistical constraints, such as those in long-term care, post-operative recovery, or mental health self-care contexts.

By facilitating modular deployment and low-barrier usage, these configurations significantly broaden the therapeutic reach of the system and enhance overall user compliance, adaptability, and outcome consistency.

### Group Therapy and Network Synchronization

### Multi-User Coordination

In another embodiment, the system is configured to support synchronized group neuromodulation therapy sessions, wherein multiple users concurrently engage in structured vibroacoustic, auditory, and visual stimulation protocols. Group therapy functionality is enabled through the integration of biometric monitoring systems and a network-based temporal synchronization architecture, allowing for both co-located and distributed therapeutic engagement.

Each user station in the group configuration comprises a modular topper positioned beneath the body to form the base of a three-dimensional vibroacoustic cocooning structure. Embedded transducer elements deliver directional vibrational energy to the body and head regions, while localized transducer pucks may be positioned adjacent to specific anatomical zones, such as the hands or feet, to provide targeted vibrational input. Physiological sensors are affixed to each participant to monitor biometric signals, such as heart rate and galvanic skin response, which inform dynamic session adjustments. Synchronized auditory stimuli are delivered to each user through headphones, temporally aligned with vibroacoustic and visual stimuli as appropriate.

The system employs a centralized control architecture responsible for managing signal timing, waveform characteristics, and delivery parameters across all user nodes. This control setup includes a primary interface for initiating and monitoring sessions, an amplifier or controller for modulating stimulus intensity and waveform integrity, and calibration components for real-time energy verification. A synchronization module distributes low-latency timing markers across the network, ensuring precise phase alignment of all stimuli among participants.

For co-located group sessions, users are physically present in the same environment, receiving stimuli from shared or individually configured components. In remote group therapy settings, users engage from different geographic locations while remaining synchronized through network-based connectivity such as Wi-Fi, Bluetooth, or proprietary low-latency communication protocols. This enables real-time interactivity and coherence across all participants, regardless of physical proximity.

The system further supports integration of acoustic sources, such as musical instruments or singing bowls, and includes environmental sensors to capture spatial and auditory data for use in real-time entrainment or playback replication. These inputs expand the sensory spectrum to include inaudible or sub-audible vibrational content, which is processed and redistributed as part of the therapeutic output.

To support shared neural synchronization, the system incorporates biometric and neurophysiological data streams, including high-density EEG, which can be used to modulate session parameters for group coherence. Environmental control modules may adjust factors such as temperature, airflow, or olfactory stimuli in real-time, coordinated across users to enhance immersive protocol delivery.

The group synchronization architecture includes redundant timing mechanisms—utilizing light, vibration, and audio markers—to correct for latency drift, jitter, and desynchronization across devices. This ensures that all sensory modalities remain phase-aligned, which is essential for efficacy in high-resolution entrainment and cognitive modulation protocols.

In population-level configurations, a distributed network of neuromapping kiosks may be deployed across various locations. These stations collect individual brainwave data, transmit it to a centralized cloud repository, and contribute to a normalized database for cohort-specific protocol refinement. This enables system-wide optimization based on large-scale neural analytics, bridging the gap between individual therapy and population neuroscience.

### Adaptive Group Feedback

In accordance with various embodiments, the system includes functionality for collecting, aggregating, and processing biometric data from multiple participants simultaneously—such data including but not limited to electroencephalographic (EEG) signals, heart rate, respiratory patterns, galvanic skin response (GSR), and skin temperature. This biometric information is conveyed to a central control module in real time for group-level analysis and adaptive sensory modulation.

A group feedback loop is established whereby the central module assesses collective biometric trends and dynamically adjusts sensory stimuli delivered to each participant. For example, when a cohort exhibits converging gamma-band activity or a shared reduction in heart-rate variability (HRV), the system may autonomously modulate vibrational frequency or amplitude to reinforce the emergent group coherence or shared emotional state.

Each individual participant is additionally equipped with physiological sensors that monitor biometric indicators—such as GSR, heart rate, or skin temperature—and feed these data into the real-time control system to optimize individual neuromodulatory responses within the group context. Headphones deliver auditory stimuli synchronized with vibroacoustic and optional visual elements. All sensory channels are phase-aligned to create a cohesive multi-sensory experience, enhancing neural entrainment and immersion across participants.

The architecture supports advanced group dynamics and cognitive-state interaction: participants are linked via high-density EEG and other biometric acquisition systems, enabling synchronized brain-body mapping and a shared neuromodulation experience that adapts both to individual patterns and collective trends. Stimuli may be adjusted so as to either converge users toward a shared state or dynamically differentiate according to individual engagement or therapeutic profiles. This design supports applications including group meditation, cognitive enhancement in classroom or corporate settings, therapeutic collectives, and research-driven multi-subject tracking and response modulation.

Moreover, the system integrates both audible and inaudible frequency components derived from live acoustic or environmental sources. Spatial mapping of frequencies is accomplished using microphones and accelerometers. These frequencies are then routed to participants via RF, Wi-Fi or wired connections. The dual-frequency integration enables enhanced realism and sensory depth—especially when engaging resonant instruments (such as singing bowls) or natural harmonic sources—and supports somatosensory stimulation by rendering sub-audible vibrations perceptible through body-mounted transducers. This enhanced method delivers a richer immersive experience, beyond auditory stimulation alone.

### Local and Remote Session Alignment

In another embodiment, the system is configured to support both co-located and distributed group therapy sessions through a low-latency communication and synchronization framework. This architecture ensures temporal alignment and consistent delivery of multimodal sensory stimuli—vibrational, auditory, and visual—across all participating user modules, regardless of their physical location.

To facilitate such alignment, the system employs one or more low-latency communication protocols, including but not limited to Bluetooth, Wi-Fi, or proprietary radio frequency (RF) transmissions. These protocols enable rapid transmission of control and synchronization signals between therapeutic modules in real time.

A central synchronization module is included within the control architecture to ensure precise phase and timing coherence across devices. This module compensates for potential transmission delays or network jitter using buffered correction mechanisms and predictive synchronization algorithms. In doing so, it maintains phase-locked stimulus delivery, thereby preserving the neuromodulatory integrity of the session, particularly in distributed or asynchronous network environments.

The control and synchronization system comprises several components:
A primary control interface, such as a tablet, smartphone, or desktop terminal, allowing a practitioner or user to configure and manage session parameters including modality selection, timing, waveform characteristics, and user-specific settings.
An amplifier or controller unit operatively connected to the control interface, responsible for modulating the output of vibroacoustic transducers and synchronized audio channels. This unit may employ digital signal processing (DSP) hardware to enable per-channel frequency, amplitude, and phase control.
A calibration and energy monitoring module configured to evaluate signal integrity and energy distribution across individual user stations. This module employs sensors—such as microphones and accelerometers—to assess vibrational amplitude, waveform fidelity, and timing accuracy, enabling real-time fine-tuning of energy delivery to maintain therapeutic consistency throughout the group session.
A synchronization and communication module, which ensures that all local and remote devices maintain coordinated timing. This module may transmit synchronization triggers embedded in data streams (e.g., EEG, audio, or video), detect deviations in timing via sensor feedback, and implement corrective adjustments using predictive modeling or fallback timing references.

This architecture enables a flexible deployment of the system for both local (co-located) and remote therapy scenarios. In local mode, multiple users situated within a single environment receive synchronized stimuli across their respective modular stations. In distributed mode, users situated in disparate locations—such as homes, clinics, or remote care environments—participate in a shared session via cloud-based or peer-to-peer networking, with synchronization maintained via the central control system.

The system also supports embedded synchronization triggers to coordinate external physiological or environmental monitoring systems. For example, time-stamped event markers may be inserted into EEG recordings to denote stimulus onset, thereby facilitating accurate segmentation of neural data for biomarker analysis or phase-locked response tracking.

In advanced embodiments, the synchronization system includes:
Redundant timing markers embedded within all sensory modalities.
Verification feedback mechanisms using sensors such as microphones and accelerometers to confirm actual physical stimulus emission.
A sensory environment control module operable to activate external environmental components, including but not limited to thermal devices, fans, lighting, or olfactory generators, via IoT protocols or direct control interfaces. These environmental elements are integrated into the session timing and may be triggered synchronously with vibroacoustic or auditory cues to enhance immersion and neuromodulatory efficacy.
A high-density EEG module may be incorporated into the system to enable real-time tracking of user brainwave activity. EEG-derived metrics may be used to inform dynamic adjustments in stimulation parameters during the session, thereby optimizing alignment with individual or group neuromodulatory goals.
Additionally, an optional AR/VR visual interface may be employed to present visual stimuli that are synchronized with the vibroacoustic and auditory channels. This interface enhances user immersion and supports therapeutic applications involving sensory integration, attentional modulation, or exposure-based protocols.

The overall synchronization infrastructure described herein allows for scalable, multi-user deployment while preserving high-fidelity timing, individualized therapeutic control, and robust integration with biofeedback and environmental systems.

### Benefits and Applications of Group Synchronization

The system's capability to synchronize neuromodulation across multiple users enables collective therapeutic experiences, yielding benefits such as shared emotional resonance, wherein participants report amplified effects by simultaneous sensory engagement, and improved user adherence—particularly in support-group, studio, or classroom environments. In addition, the group-synchronization functionality opens novel research and clinical applications for investigating collective entrainment, social coherence phenomena, and the neurophysiological impacts of group-based wellness interventions.

By virtue of this group functionality, the system functions not only as an individual therapeutic device but also as a scalable platform for collective neuromodulation within communal, group-therapeutic, or institutional settings. Such environmental orchestration is particularly valuable in applications requiring high levels of emotional or neurological immersion, for example: post-traumatic stress disorder (PTSD) extinction training, cognitive-behavioral desensitization protocols, immersive meditation or virtual-ritual experiences.

Moreover, the system combines neural-data acquisition, multi-sensory feedback, and environmental control into a comprehensive neuromodulatory framework. This integrated architecture is technically robust—providing precise timing, phase-alignment and adaptive control—and clinically versatile, capable of adapting to diverse therapeutic contexts, multi-user configurations, and longitudinal wellness programs.

In group therapy and kiosk deployment scenarios, the system is configured for networked multi-user operation—wherein multiple therapy mats or transducer modules are synchronized through a central controller to enable shared or individualized neuromodulation sessions guided by real-time biometric data. Each user station is equipped with the necessary hardware and software to support low-latency communication and synchronization, including wireless protocols (Bluetooth, WiFi, RF) between mats and the central controller, broadcast of master timing signals to align vibrational, auditory and visual stimuli across participants, and bidirectional data flow for user-specific EEG, biometric or calibration data back to the controller. Synchronization ensures that shared sensory cues (for example 40 Hz stimulation) occur simultaneously across all user stations, thereby facilitating cohesive group entrainment and uniform therapeutic impact.

The kiosk-style controller interface provides a dashboard view of all active mats and users, session scheduling and configuration tools for group protocols, biometric monitoring windows (displaying live EEG, heart rate, or skin conductance from multiple users), and automated alerts or calibration prompts if synchronization anomalies or module faults are detected. The system supports operation in manual, semi-automated or fully automated modes, as suited to practitioner preference and therapeutic context.

Adaptive group feedback protocols are supported, wherein biometric data from the cohort is collected and aggregated to guide session adjustments: for example, EEG coherence across users may trigger modulation of vibrational amplitude or frequency; the system may identify biometric outliers (such as a user exhibiting stress responses) and deliver personalized modulation to that user while maintaining group-wide synchronization; group metrics such as total heart-rate variability or gamma-band synchrony inform collective pacing, emotional regulation, or relaxation timing. Thus the system operates not only as a multi-sensory stimulation tool but also as a social neuromodulation platform.

Configurable deployment scenarios include yoga or meditation classes featuring synchronized mats aligned to instructor cues; clinical sessions where patients receive tailored entrainment within a shared therapeutic framework; research studies capturing synchronized EEG and vibrational data across multiple users for population-level analysis. Portable group-kits may include multiple foldable mats, a group controller and necessary EEG headsets or biometric sensors, configured for rapid setup and breakdown in mobile environments.

Session logging and data analytics are integrated within the system: per-user session metadata (including duration, frequency bands used, EEG responses), group-level synchronization metrics and phase-alignment quality, and practitioner notes or post-session outcome assessments are recorded. This dataset supports refinement of therapy protocols, assessment of group dynamics, and investigation of emergent neurophysiological synchrony trends or collective wellness outcomes.

### Sleep and Meditation Ecosystems

In accordance with various embodiments, the system is configured for use in individualized vibroacoustic therapy contexts, particularly those oriented toward meditation, guided wellness routines, or sleep-related applications. The architecture supports both independent personal use and networked sessions (e.g., remote or group-synchronized) while maintaining core capabilities of real-time feedback, calibration, and modality integration.

In one implementation, the therapy surface is adapted to a modular meditation cushion designed as an active seating structure incorporating embedded vibroacoustic transducer elements. The cushion is configured for seated postures and may be used in settings such as yoga studios, meditation centers, private homes, or as part of a networked virtual session. The embedded transducers are disposed beneath the user and oriented to direct vibrational energy upward along anatomical axes such as the spinal column or pelvic region. The system permits dynamic modulation of energy parameters, including amplitude, frequency, wave-form pulse pattern, and spatial zoning, in alignment with the session's therapeutic goal.

Use cases include, but are not limited to:
Classroom settings, wherein an instructor may activate the transducer element to deliver subtle vibroacoustic feedback to selected participants without drawing overt attention, thereby supporting focused attention or group synchronicity;
Remote wellness or tele-therapy protocols, in which guided meditation or sleep induction sessions are coordinated via networked control, delivering personalized stimulation with real-time biometrics and optional group synchronization.

A calibration module is positioned adjacent to the user, enabling monitoring and adjustment of vibrational output. The module analyzes posture, body contact zones, user anatomy and cushioning interface to ensure optimal coupling and energy delivery efficacy. The calibration module may operate autonomously via embedded algorithms or under practitioner supervision for fine-tuned adjustment. The user additionally wears headphones to deliver synchronized auditory stimuli that are phase-aligned with vibrational feedback from the cushion, thereby forming a multi-sensory entrainment experience. This integration enhances immersion, promotes neural coherence, supports emotional regulation, and improves cognitive focus.

The meditation-cushion configuration supports both standalone personal sessions and participation in broader networked ecosystem sessions. Even in solo use, the user's session may be recorded, biometric and calibration data logged for future personalization, and optional synchronization established with remote participants for hybrid or group engagement.

By enabling vibroacoustic treatment in seated meditation and sleep-transition contexts, the system broadens therapeutic versatility. It offers a low-barrier, self-administered format while retaining full capability for calibration, biometric integration, and networked synchrony-thereby promoting adherence, flexibility, and continuous wellness support.

### Acoustic Instrument Integration

In accordance with certain embodiments, the system is configured to integrate with live acoustic musical instruments to enhance the neurosensory and therapeutic value of vibroacoustic sessions. This integration allows for real-time capture, processing, and transmission of both audible and sub-audible frequencies generated by acoustic instruments, which are then rendered through the system's transducer array as synchronized body-coupled vibrational stimuli.

In one implementation, the system incorporates one or more acoustic capture devices, including high-sensitivity microphones, contact microphones (piezoelectric or otherwise), accelerometers, or surface vibration sensors, positioned in proximity to or in physical contact with an acoustic musical instrument. Examples of suitable instruments include, but are not limited to, piano, violin, cello, harp, acoustic guitar, singing bowls, and percussive instruments. These sensors are configured to extract a broad frequency spectrum from the instrument, including both audible sound and low-frequency vibrations that may fall below the threshold of human auditory perception.

Captured signals are routed through a signal processing subsystem, which may include analog-to-digital conversion, digital filtering, frequency band isolation, and gain adjustment. The processing unit distinguishes between frequency ranges suited for auditory output and those suitable for vibroacoustic transduction. The former are directed to auditory modules (e.g., headphones, speakers), while the latter are converted into control signals for the system's vibroacoustic transducer array.

The transducer array, which may be embedded in a therapeutic mat, wearable module, or cushion, is driven in real time by the processed vibrational content of the instrument. This results in synchronous delivery of body-coupled vibration aligned with the acoustic performance. The system may also apply spatial mapping algorithms to modulate transducer output based on harmonic content, dynamic amplitude, or instrument articulation characteristics.

This configuration enables immersive, multi-sensory experiences where live musical expression is rendered not only through sound but also as physically felt vibration. Applications include:
Therapeutic music sessions in clinical or wellness environments.
Live performance enhancement, enabling performers or audience members to experience music as full-body tactile immersion.
Cognitive or emotional modulation, wherein musical motifs are designed to evoke targeted affective states and are reinforced through synchronized vibratory coupling.
Real-time entrainment protocols, using rhythmic or tonal features of live instruments to modulate brain and body states.

In some embodiments, the system includes spatial microphones or 3D audio cameras to capture positional and directional data, enabling further refinement of the vibroacoustic rendering to match spatial characteristics of the sound source. This may support dynamic adaptation of vibrational fields to reflect changes in performer movement or acoustic environment.

Through acoustic instrument integration, the system extends its neuromodulatory platform to include live, expressive content from traditional musical modalities, enriching both therapeutic and experiential applications.

### Immersive and Therapeutic Scenarios

In accordance with various embodiments, the system is configured to integrate with live or recorded acoustic sources, spatial sensing, and environmental modules to create immersive, multi-sensory therapeutic scenarios. These embodiments combine vibroacoustic stimulation, auditory realism, somatosensory feedback, and spatially aware capture/replay systems to support applications ranging from deep meditation and wellness to therapeutic rehabilitation and group immersion.

In one implementation, resonant acoustic instruments—such as a singing bowl—are used as primary sources. The singing bowl generates both audible frequencies and sub-audible harmonic content. The system captures the rich harmonic output and directs low-frequency components through the vibroacoustic transducers, thereby coupling vibration into the user's body and activating somatosensory receptors in addition to auditory pathways. This creates a layered sensory experience in which both audible sound and felt vibration converge to increase user immersion.

Spatial capture is achieved using arrays of microphones and cameras configured to record three-dimensional audio-visual data. Directional audio cues enable the reconstruction of a full 3D soundfield for playback, while visual data allow participants to engage in virtual reality (VR), augmented reality (AR) or standard display environments with realistic movement and acoustic feedback. In this way, the system facilitates realistic participation in immersive therapy environments—useful for remote group sessions, guided meditations, or recreational wellness applications where environmental fidelity influences emotional and neurological impact.

Additional acoustic inputs—such as a piano—are also integrated. Whether performed live or via recorded content, the piano contributes broad-spectrum harmonic energy. The system processes these inputs and routes sub-audible frequencies to the user's body via transducers, thus expanding the sensory spectrum beyond audible sound. This embodied listening experience leverages the user's capacity to perceive sub-audible vibrations through the body, offering enhanced neural engagement.

Together, this architecture enables:
Integration of multiple live or pre-recorded sound sources into the therapy environment;
Translation of rich acoustic environments into somatic and auditory signals;
Capture and reproduction of experiences in spatially aware, immersive formats (audio, vibration, visual).

These capabilities enhance the therapeutic and emotional depth of the system, allowing for complex, expressive sessions that can be personalized, shared, replayed, or adapted to therapeutic objectives.

Furthermore, the system includes companion and environmental modules to extend its reach beyond human users:
Pet-focused vibroacoustic modules incorporate smaller mats or cushions embedded with low-frequency transducers safe for animals (e.g., dogs, cats), optionally synchronized with a human user to deliver shared therapy, anxiety reduction, or rest-state support in service or recovery settings.
Plant and bioelectric signal modules utilize sensors that detect subtle voltages or bioelectric fluctuations in plant tissues and translate them into vibroacoustic or auditory outputs, supporting biophilic entrainment, ambient sensory feedback, or nature-integrated therapy.
Ambient environmental synchronization modules monitor external conditions such as sound level, light intensity, air quality, barometric pressure or human motion and adapt session parameters accordingly (e.g., increasing vibroacoustic intensity during noise spikes, synchronizing session start with circadian cues such as dawn light, activating thermal or olfactory feedback via smart devices).

These companion and environmental modules:
Interface with the primary control software via wired or wireless channels;
Are configured via the same user-interface or session templates;
Operate in either passive data-collection or active bidirectional feedback modes;
Enable multi-species, multi-sensory environments optimized for relaxation, rehabilitation, or exploratory entrainment of environmental systems.

### Alternative Configurations and Non-Traditional Applications

In accordance with additional embodiments, the modular and foldable vibroacoustic mat design is adapted to a variety of alternative configurations and environments beyond traditional body-work or clinical therapy settings. These use-cases extend the versatility of the system into personal wellness, group therapy, interspecies support, and environmental or biophilic applications.

### Portable Meditation Seat

The foldable mat is configured into a chair-like format suitable for use in home, travel, or mobile contexts. One or two transducer pucks are mounted behind the user's lower back or beneath the seat surface to deliver subtle vibrational stimulation. This compact setup is optimized for guided breath-work, mindfulness meditation, or application-based neurofeedback programs that synchronize auditory cues with vibroacoustic stimulation.

### Group Therapy Kiosk

A plurality of foldable mats are arranged in side-by-side configuration to support group therapy or community wellness experiences (e.g., yoga studios, spa clinics, sound-healing rooms, meditation centers). Each mat is connected either to a local control module or networked centrally through wired or wireless links. The system supports synchronized group protocols or individualized therapeutic tracks within the group environment, enabling both collective entrainment and differentiated stimulus delivery.

Pet and Plant Modules. Smaller-format vibroacoustic pads are adapted for non-human use cases:
Pet Module: A downsized mat is calibrated to deliver low-intensity calming vibrations appropriate for animals (e.g., domestic pets or veterinary recovery contexts). The module may support anxiety reduction, calming protocols, or rest-state support during human treatment sessions.
Plant/Bio-feedback Module: Sensor modules measure real-time bio-electrical signals from plants (e.g., leaf conductivity, soil impedance). These signals are converted into sub-audible hums or modulated sonic patterns and delivered via vibroacoustic output, extending system application into biophilic and ecologically attuned environments in which human users may entrain with living systems.

These alternative configurations demonstrate that the system is not limited to traditional full-body therapeutic tables, but can be scaled down or adapted for discrete, portable, wearable, group, animal, and environmental settings. This flexibility enhances session accessibility, supports diverse user populations, and enables integration into lifestyle, wellness, ecological, or multi-species therapeutic frameworks.

### Extended Modalities and Therapeutic Application

### Transcranial Electrical Stimulation (TES) and Transcranial Magnetic Stimulation (TMS) Integration

In accordance with various embodiments, the system extends its neuromodulatory platform by integrating components for delivering Transcutaneous Electrical Stimulation (TES) and Transcranial Magnetic Stimulation (TMS). These modalities enable direct electrical and magnetic stimulation of peripheral and central neural targets, thereby extending therapeutic capability beyond sensory-based entrainment to active neuromodulation of neural circuits.

### TES and TMS Electrode and Coil Arrays

The system supports a plurality of stimulation modules including handheld or attachable electrode arrays for TES and positionable magnetic coil assemblies for TMS. These modules may be embedded within the modular therapy apparatus or used as auxiliary devices depending on the therapeutic context. Exemplary target sites and functions include:
Trigeminal Nerve Stimulation (TES): Electrode arrays configured for application to the forehead or cheeks targeting branches of the trigeminal nerve. This application supports autonomic modulation, cranio-facial pain management, and mood regulation via trigeminal pathways to brainstem and thalamus.
Vagus Nerve Stimulation (TES): Electrode arrays positioned adjacent the cervical neck region or auricular concha to stimulate the vagus nerve, thereby modulating parasympathetic tone, reducing sympathetic overactivity, and supporting stress recovery and emotional regulation.
Median Nerve Stimulation (TES): Arrays applied to the forearm are used to deliver stimulation to the median nerve, facilitating sensorimotor entrainment, neuroplastic enhancement, and analgesic outcomes in pain rehabilitation.
Glossopharyngeal Nerve Stimulation (TES): Electrodes placed near the upper neck or jawline target the glossopharyngeal nerve to regulate airway control, respiratory rhythm, and conditions such as sleep apnea.
Peripheral Muscle and Nerve Stimulation (TES): TES modules may stimulate limb muscles or peripheral nerves to support motor cortex activation, stroke rehabilitation, or extinction training by coupling motor output and sensory feedback.

Transcranial Magnetic Stimulation (TMS): The system optionally includes non-invasive magnetic coil assemblies for delivering focal or distributed magnetic pulses to the scalp, thereby inducing electrical fields in underlying cortical tissue. Such capability enables:
1. Modulation of cortical excitability targeting brain regions implicated in depression, anxiety, and cognitive performance.
2. State-dependent stimulation synchronized to user-specific brain states detected via high-density EEG (HD EEG) or neuromap profiling.
3. Adjunctive use with sensory entrainment modalities (vibroacoustic, auditory, visual) to disrupt maladaptive network connectivity or enhance coupling and entrainment.

### Customization and Safety Controls

Each stimulation module (TES or TMS) supports programmable control of parameters including pulse width, stimulation frequency, amplitude, timing, and waveform shape. Control may be manual or automated based on real-time biometric or neurophysiological feedback. Embedded safety protocols ensure stimulus parameters remain within clinically safe thresholds and engage fail-safe mechanisms in instances of abnormal readings or sensor disconnection. The integration of these TES and TMS modules extends the platform's therapeutic scope, enabling layered neuromodulation pathways for simultaneous modulation of peripheral and central neural systems. These modules may operate in isolation or coordinated with other entrainment modalities (vibroacoustic, auditory, visual), supporting adaptive, individualized, and state-responsive intervention strategies.

### Use of TES and TMS for Neural Entrainment and Optimization

In embodiments where entrainment is targeted, TES and TMS are employed to facilitate direct neural entrainment and modulation of brainwave activity, either standalone or in conjunction with sensory stimulation. These modalities allow induction or reinforcement of oscillatory synchronization within central nervous system circuits by delivering stimulation pulses at user-specific or protocol-defined frequencies, thereby aligning endogenous brain rhythms with exogenous inputs and enhancing functional connectivity, neural excitability regulation, and neuroplastic change.

### Entrainment Objectives by Frequency Band

Cognitive Enhancement (TES/TMS): Stimulation at alpha (8-13 Hz) or gamma (30-80 Hz) frequencies is employed to boost working memory, attention, and learning. Synchronized sensory inputs (vibration or auditory) may further enhance stimulus-driven coherence in task-relevant neural networks.

Stress and Anxiety Reduction (TES/TMS): For emotional regulation or relaxation protocols, stimulation at theta (4-7 Hz) or low-alpha (8-10 Hz) frequencies may be applied. TES targeting vagus or trigeminal pathways complements cortical entrainment, while TMS directed at prefrontal or insular regions enhances mood regulation.

### Multimodal Synergy and Coordination

The system enables synchronous operation of TES and TMS with other sensory modalities:
Vibroacoustic transducers deliver synchronized tactile pulses matching frequency and phase of the electrical/magnetic stimulation.
Auditory tone generators or music embed rhythmic patterns aligned with neural and vibroacoustic stimulation frequencies.
Visual flicker modules or AR/VR systems present optical pulses or immersive stimuli aligned in phase and timing with the electrical/magnetic and vibroacoustic outputs.

Coordination across these modalities is managed via synchronization and latency-compensation modules to ensure precise delivery and convergence of multi-sensory input. This cross-modal integration amplifies neural engagement and enhances the range, precision and individualization of therapeutic protocols. Stimulation parameters such as pulse timing, waveform shape, inter-stimulus interval, and intensity may be defined via clinician input, session templates, or real-time feedback from biometric or EEG monitoring systems. The inclusion of TES and TMS modalities thereby enables layered, state-specific neuromodulation that is responsive to user physiological context, environmental conditions, and therapeutic intent.

### Neurochemical Modulation Interfaces

In accordance with various embodiments, the system further includes functionality to interface with neuromodulatory agents—whether pharmacologic, nutraceutical, or metabolic compounds—that modulate neurotransmitter systems. These agents are used in conjunction with the system's neurostimulation modalities (including TES, TMS, vibroacoustic, auditory, and visual stimuli) to amplify, guide, or stabilize neural responses. By pairing specified neuromodulatory agents with targeted stimulation protocols and high-density EEG (HD EEG) feedback, the system enables neurochemical-state-responsive neuromodulation, thereby supporting real-time entrainment optimization aligned to individual neurophysiological profiles.

### Targeted Neurotransmitter Modulation

Cholinergic Enhancement (Acetylcholine): The system may be used with agents that potentiate acetylcholine activity in order to improve vagal tone, attention regulation, and cognitive performance. In such embodiments, TES applied to the vagus nerve and cortical TMS targeting prefrontal regions may be synchronized with elevated cholinergic activity, thereby amplifying entrainment effects and facilitating integrative autonomic-cortical modulation.

Noradrenergic Activation (Norepinephrine): Stimulation protocols (via TES or TMS) may be combined with agents that enhance norepinephrine availability or receptor sensitivity to modulate arousal thresholds, selective attention, and executive functioning. In these cases, the system's frequency and phase alignment of stimulation pulses may be timed to coincide with the arousal curve produced by noradrenergic activation, thereby enabling precision entrainment under heightened cortical excitability.

High-density EEG source localization supports real-time monitoring of neuromodulatory effects. Metrics such as changes in oscillatory activity, phase-locking value (PLV), and functional connectivity are tracked and used to dynamically adjust stimulation parameters including intensity, pulse timing, spatial targeting, and inter-modality synchronization.

### Potential Applications and Benefits

Cognitive and Emotional Regulation: The system may deliver TES or TMS to targeted brain regions (e.g., dorsolateral prefrontal cortex, insula) while concurrently administering entrainment-aligned sensory stimuli. This multimodal approach can improve working memory, sustain attention, and stabilize mood-particularly in users with affective or attentional dysregulation.
Sleep Apnea Management: In a respiratory regulation application, TES may be delivered to the glossopharyngeal nerve to support airway patency, while TMS is directed at cortical regions involved in respiratory timing or autonomic regulation. Concurrent entrainment via vibroacoustic or auditory stimuli may support sleep-state-specific neuromodulation, potentially reducing apnea episodes and improving sleep architecture.
Pain Management and Sensorimotor Recovery: The system can administer TES to peripheral nerves (e.g., the median nerve) and simultaneously use TMS to modulate somatosensory or motor cortices. In conjunction with sensory entrainment, this coordinated intervention may reduce pain perception, stimulate motor pathways, and promote neuroplastic recovery in conditions such as chronic pain, neuropathy, or post-stroke rehabilitation.

By employing neuromodulatory agents in synergy with sensory and neurostimulation modalities, the system enables pharmacologically-informed neuromodulation protocols that are adaptive to a wide range of therapeutic contexts and personalized for each user's unique neurophysiological and biochemical state.

### Combined Entrainment and Neuromodulation Strategies

In various embodiments, the system integrates sensory-based entrainment techniques (e.g., vibroacoustic, auditory, and visual stimulation) with direct neuromodulatory interventions (e.g., EEG-guided Transcutaneous Electrical Stimulation (TES) and Transcranial Magnetic Stimulation (TMS)). This multimodal approach facilitates both bottom-up and top-down modulation of neural networks, enabling more precise, durable, and individualized therapeutic outcomes.

### Method of Combined Application

The system is configured to deliver synchronized entrainment and neuromodulation according to a defined sequence, co-activation schedule, or adaptive feedback loop. Typical configurations include:
Sequential Mode: Sensory entrainment (e.g., auditory rhythmic cues, vibroacoustic pulses) is delivered first to prepare the nervous system and increase cortical receptivity. This is followed by TES or TMS to reinforce or elevate specific neural states.
Simultaneous Mode: Vibroacoustic, auditory, and/or visual stimulation is delivered in precise temporal alignment with TES or TMS pulses, ensuring multimodal convergence on targeted neural substrates. Synchronization modules control phase and frequency alignment across modalities.

Adaptive Feedback Mode: HD EEG and biometric sensors track real-time neurophysiological states (e.g., oscillatory power, coherence, heart rate variability). Based on detected patterns, the system dynamically adjusts: TES or TMS pulse parameters (e.g., frequency, amplitude, duration). Entrainment stimuli timing, waveform, and spatial targeting. Session length and progression stages to match user-specific responses.

### Timing and Sequencing Considerations

The efficacy of multimodal neuromodulation is influenced by the relative timing and phase relationship of stimuli. To optimize this, the system:
Embeds precise timing triggers within all output channels (vibration, audio, light, TES/TMS).
Utilizes phase-locking algorithms to ensure synchronization with endogenous brain rhythms, particularly when targeting frequency bands such as delta, theta, alpha, beta, or gamma.
Allows programmable sequencing such that TES pulses, for instance, may occur at the peak of an alpha wave cycle, or TMS bursts may align with gamma phase-locked auditory inputs.

This timing alignment enhances neuroplastic engagement, minimizes neural interference, and supports state-dependent efficacy.

### Safety and Control Features

To maintain user safety and system integrity during combined entrainment and neuromodulation protocols, the system incorporates multiple protective mechanisms:
Pre-Session Calibration: All output modules (transducers, light, audio, TES/TMS) undergo system checks to ensure correct placement, electrical integrity, and alignment with user anatomy.
Dynamic Parameter Adjustment: Stimulation parameters are continuously evaluated against safety thresholds based on real-time EEG and biometric readings. If adverse patterns (e.g., excessive arousal, abnormal EEG waveforms, electrode disconnection) are detected, the system: Automatically reduces output intensity. Pauses stimulation. Issues alerts to the user or supervising practitioner.
Fail-Safe Redundancy: Independent timing systems and monitoring modules ensure synchronization persists even in the event of minor network or hardware disruptions.

### Clinical and Performance Benefits

By uniting sensory entrainment with direct neuromodulation, the system supports:
Enhanced neural phase coherence across sensory and cognitive circuits.
Improved efficacy in cognitive enhancement, emotional regulation, and neuromotor rehabilitation.
Greater user adaptability, allowing entrainment protocols to be tailored not only by sensory preference but by cortical state and neuromodulatory readiness.

This combined strategy allows the system to function as an integrated neurosensory modulation platform capable of both inducing and stabilizing target neural states across a wide range of applications.

### Personalized Multi-Modal Sensory Ecosystems

In accordance with various embodiments, the system provides a flexible and modular sensory-integration ecosystem designed to deliver personalized, brain-map-driven stimulation across multiple sensory modalities—including vibration, sound, and light. At the core of this ecosystem is a high-definition neuromap, which guides the creation of user-specific spectro-temporal patterns optimized for neural entrainment or desynchronization. This architecture supports both stationary and mobile application scenarios, enabling users to benefit from individualized protocols in spa sessions, therapeutic treatments, commuting environments, or virtual/augmented-reality platforms.

### System Workflow

Rapid HD Neuromap Acquisition: The system performs high-resolution brainwave recording (e.g., via HD EEG) to capture key features of the user's neural activity across frequency bands such as alpha, gamma, theta and others.

Personalized Spectro-Temporal Pattern Generation: Based on the recorded neuromap, the system generates custom stimulation patterns—for example specific vibration frequencies, auditory rhythms, and light-flicker sequences—tailored to the user's individual neural profile.

Multi-Sensory Integration and Delivery: The generated patterns are embedded into synchronized outputs across the vibroacoustic, auditory and visual channels. This multi-modal delivery ensures coherent stimulation of the user's sensory and neural systems.

### Modular Component Infrastructure

Transducer Cushions or Pucks: Modular vibrational elements tuned to the individualized neuromap data. These may be arranged on spa tables, meditation seats, beds or foldable mats to create an enveloping vibroacoustic “cocoon” effect.
Headphones and Speakers: Acoustic output modules deliver sound driven by the personalized spectro-temporal mapping, synchronised in timing and phase with vibrational stimuli.
Vision Module: A visual stimulus subsystem using flicker-based lights, VR/AR interfaces or strobing modules, synchronized with vibration and sound to enhance immersive neural engagement.

### Synchronization and Calibration

The system further incorporates synchronization modules to ensure phase and latency alignment across modalities. A handheld calibration device, which includes an accelerometer and spectrogram display, allows verification and on-the-fly adjustment of vibrational amplitude, frequency fidelity, and spatial distribution. These calibration components ensure that the delivered vibration, sound and light faithfully match the neural targets derived from the neuromap, regardless of environment or configuration.

### Portable and On-the-go Configurations

Portable Delivery Module: A pocket-sized unit comprising a compact transducer and control electronics, capable of delivering neuromap-driven vibration (e.g., personalised ~40 Hz output) in mobile contexts.
Wearable Multi-Sensory Devices: Body-worn modules such as wristbands, hats, glasses or apparel embedding vibration, light flicker and audio in discrete form factors for daily mobility.
Large-Scale Synchronisation: A wireless transmitter/receiver system supports synchronization of up to many devices simultaneously (e.g., in group or distributed therapy sessions).
Dynamic Calibration Modules: Mobile calibration assemblies enable verification of vibrational fidelity across environments—for example different seating surfaces, vehicle seats or public spaces.

### Personalized Experience and Neural Integration

By leveraging the individualized neuromap and delivering synchronized multi-modal stimuli, the system facilitates deep neural entrainment or desynchronization below the threshold of conscious perception. Users may experience relaxation, effective stimulation, focus enhancement or therapeutic benefit based on the tailored protocol. This combination of experiential immersion and neurological optimization expands the system's applicability across therapeutic, cognitive enhancement, wellness and lifestyle domains.

### Population-Level Brain Mapping and Cloud-Based Neuromodulation Optimization

In accordance with various embodiments, the system comprises a network-integrated brain-mapping infrastructure designed to support population-scale data collection, normalization, and analysis of neuromodulatory biomarkers, thereby enabling large-scale optimization of entrainment and stimulation protocols.

### Population Brain Mapping Infrastructure

A plurality of distributed rapid brain-mapping kiosks are deployed across diverse locations. Each kiosk is equipped with high-density electroencephalography (HD EEG) systems for high-resolution brain activity capture, spatial imaging apparatus for anatomical alignment and stimulus targeting, and multi-modal sensory integration modules (vibroacoustic, auditory, visual) to deliver real-time stimuli and record responses.

These kiosks are interconnected via secure data transmission protocols, uploading neuromap data in real time to a centralized cloud infrastructure. The data pipeline supports high-frequency updates, permitting neuromap snapshots from thousands of users to be ingested and processed rapidly. The cloud repository provides scalable storage with built-in versioning, user metadata tagging (e.g., timestamp, session context, geographic location, demographic parameters), and encryption and access-control mechanisms to safeguard user privacy and data integrity.

Within the cloud database, neuromap data are normalized and organized to support cross-sectional and longitudinal analysis across heterogeneous populations. Features include geographic and demographic segmentation, cross-time integration enabling mapping of dynamic neurological shifts across hours, days or longer time spans, and standardization of neural features to provide baseline comparisons and outlier detection.

### Population-Wide Change Detection and Protocol Optimization

The system supports temporal population-level analysis by collecting neuromap data at discrete intervals (e.g., time 1, time 2, time 3) and comparing across cohorts to detect collective neurophysiological trends. Resulting insights include: changes in neural synchronization patterns across groups, variations in brain-wave activity within and between demographic segments, and emergent biomarkers indicative of shifts in cognitive, emotional or circadian states.

A population change report is generated summarizing key findings and may inform: adjustments to existing entrainment or stimulation protocols, development of new population-specific neuromodulation strategies, and detection of emerging neural trends relevant to public health, cognitive performance, or environmental stressors.

Further, the system supports tailored protocol development by comparing individual neuromap signatures or group deviations with the normalized neural database. Using this method, the system may: identify sub-groups with atypical neural signatures, recommend modified stimulus delivery patterns aligned with detected biomarker profiles, and adapt therapy parameters based on circadian rhythm alignment, regional variation or demographic-specific entrainment profiles. For example, if a population afflicted with circadian-related waveform deviations is identified, the system may adjust timing or frequency of sensory input to improve entrainment efficacy. Alternatively, group-level biomarkers (e.g., a shift in alpha-band synchrony across a cohort) may trigger proactive modifications in wellness strategies or neuroadaptive protocols.

Through this architecture, the system evolves into a feedback-driven, biomarker-aware neuromodulation platform capable of continuously refining itself based on real-world, population-wide data streams.

### Sensory Integration Ecosystem Architecture

In accordance with various embodiments, the system comprises a modular sensory-integration ecosystem designed to deliver neuro-customized, multi-modal entrainment stimuli-including vibroacoustic, auditory, visual, and auxiliary sensory channels-based on high-resolution neural profiling. This architecture supports both stationary and mobile use cases, facilitating tailored neuromodulation protocols in clinical, home, group, or portable settings.

### System Workflow

Rapid High-Definition Neuromap Acquisition: The process begins with generation of a high-density EEG-based neuromap that captures the user's spatio-temporal brain-activity profile. Advanced signal-processing algorithms identify individualized neural characteristics such as the user-specific gamma-peak frequency, alpha-band power distribution, theta-gamma coupling metrics, and other relevant biomarkers.

Pattern Generation and Modality Mapping: Based on the neuromap metrics, the system generates personalized spectro-temporal stimulation patterns for each modality—vibration, sound, light, and auxiliary senses. These patterns are embedded into modular outputs to target either entrainment (synchronization) or desynchronization (reset) of specific neural circuits or frequency bands.

Multi-Modal Delivery and Integration: The modular architecture routes the generated patterns into the vibroacoustic transducer cushions or pucks, synchronized auditory output devices (headphones or speakers), and visual modules (flicker lights, VR/AR interfaces). Auxiliary sensory modules—such as thermal, olfactory, ambient flow or haptic overlays—may also be integrated. These modalities are synchronized in time and phase to maximize neural convergence and controlled modulation of the user's brain state.

Dynamic Re-configuration Across Use-Cases: The ecosystem supports deployment in diverse contexts including clinical therapy tables, home wellness platforms, group-therapy studios, or mobile/wearable setups. Modular components can be added, removed or re-positioned to adapt to anatomical targets, environmental constraints, or user mobility. The synchronization and calibration subsystems ensure that each configuration maintains phase integrity, latency compensation, and user-specific fidelity regardless of setting.

### Architecture Objectives

Facilitate targeted entrainment: enhancing synchrony in desired brainwave bands (e.g., individualized gamma, alpha) to support cognitive enhancement, focus, or neuro-rehabilitation.

Enable controlled desynchronization: intentionally disrupting or resetting maladaptive neural patterns for cognitive flexibility, mood regulation, or therapeutic intervention.

Maintain modality convergence: delivering tactile, auditory, visual and auxiliary stimuli in phase and frequency alignment to engage multiple sensory-neural pathways and promote deeper neuromodulatory effect.

Provide environmental and contextual adaptability: designed for clinical, home, group, and mobile uses, with reconfigurable hardware modules and seamless synchronization across modalities and settings.

### Personalized Multi-Modal Sensory Ecosystem

In accordance with various embodiments, the system is configured to perform source triangulation of neural oscillatory activity—thereby identifying not only surface EEG characteristics but also depth-level neural origins of specific brain rhythms. This capability enables deeper, more precise targeting of entrainment or desynchronization protocols, enhancing therapeutic specificity and efficacy.

### Source Triangulation and Neuromap-Driven Pattern Construction

Based on the individualized neuromap, the system constructs personalized spectro-temporal stimulation patterns configured to modulate selected neural oscillations. These patterns may include, but are not limited to:
Amplitude-modulated vibroacoustic pulses precisely aligned to the user's neural resonance frequencies.
Binaural beat audio tracks or musical compositions embedded with envelopes matching the user-specific target frequency bands.
Pulsed light stimuli, such as stroboscopic flashes or hue-shifting visual sequences, delivered in precise temporal sync with EEG-identified rhythmic patterns.

These individualized stimuli may be applied either to entrain desired oscillatory states (for example enhancing cognitive focus via mid-gamma frequencies) or to disrupt maladaptive neural synchrony (for example reducing excessive alpha band coherence associated with rumination or anxiety).

Multi-Sensory Integration with Precise Synchronization

All sensory outputs—vibroacoustic, auditory, and visual stimuli—are integrated with exacting phase alignment and latency-controlled synchronization to ensure concurrent and in-phase arrival at the user's sensory and neural systems. The synchronization process accounts for:
Peripheral transmission delays, such as latency introduced by wireless headphone transmission, speaker buffering, or remote transducer arrays.
Neural transmission latencies derived from individual latency-mapping sessions (as described previously), enabling stimulus delivery timing that aligns with user-specific neurophysiological conduction delays.

By compensating for such delays and orchestrating simultaneous delivery, the system maximizes neural coherence, perceptual unity, and entrainment effect. The resulting immersive, harmonized, neuro-adaptive therapeutic experience is customizable in real time and extendable across a diverse range of deployment environments—stationary or mobile.

In one embodiment, the system comprises a stimulation assembly (the “multi-layer stimulation assembly”), a craniofacial bone-conduction bar mechanically coupled to a cranial or mandibular surface, one or more robotic actuators or positioning arms, a neural-signal acquisition unit comprising a high-density electroencephalography (HD-EEG) sensor array, a computing system executing a personalization algorithm, and a control interface enabling closed-loop modulation between EEG activity and the multi-modal stimulation outputs.

The multi-layer stimulation assembly is arranged to deliver vibroacoustic, photonic, and optionally transcranial alternating current stimulation (tACS) to a user. The assembly is mounted or held in spatial orientation relative to the user's head, face or skull via the craniofacial bar, and the robotic actuators or positioning arms control its spatial orientation and applied contact force. The neural-signal acquisition unit continuously monitors the user's brain activity, transmitting real-time data to the computing system. The computing system applies the personalization algorithm to learn from the EEG data and user responses over time in order to adjust stimulation parameters. The control interface presents session configuration, real-time feedback and permits closed-loop modulation such that stimulation parameters are adjusted based on neural responses.

### Multi-Layer Stimulation Assembly

The multi-layer stimulation assembly comprises at least three functional layers: A vibroacoustic layer configured to generate mechanical vibration, typically delivered via contact to cranial, mandibular, or craniofacial bone structures (via the bone-conduction bar). In certain embodiments this vibroacoustic layer operates within a frequency spectrum of approximately 20 Hz to 300 Hz, selected to couple via bone conduction resonance to cranial structures and transmit tactile and somatosensory stimulation to the user.

A photonic layer configured to emit modulated light in the near-infrared or visible spectrum (for example 400 nm to 900 nm). This photonic emission is synchronized to the vibratory harmonics so that the light pulses or waveforms correspond in timing and phase to the vibroacoustic stimulation, thereby enhancing multi-sensory entrainment.

An optional transcranial alternating current stimulation (tACS) subsystem configured to apply phase-locked electrical currents to the user's scalp and underlying cortical structure. In embodiments, the tACS subsystem applies currents at frequencies in the range of 1 Hz to 40 Hz, with the phase aligned to oscillatory peaks or troughs detected in the EEG signal of the user. The tACS circuitry is integrated into the stimulation assembly or may be a separate attachable module.

### Craniofacial Bone-Conduction Bar and Robotic Actuators

The craniofacial bone-conduction bar is mechanically coupled to at least one cranial or mandibular surface of the user—such as the mastoid bone behind the ear, the zygomatic arch, or the mandibular ramus. The bar provides a rigid yet compliant interface through which vibroacoustic energy is delivered directly to bone conduction pathways. In certain embodiments, the bar contains an embedded vibration transducer and contact sensors to ensure coupling quality.

Positioning and contact force of the stimulation assembly are controlled via one or more robotic actuators or positioning arms. These arms typically provide six degrees of freedom (6-DOF) and include force-feedback control. The actuators enable precise spatial orientation—pitch, yaw, roll, translation in x/y/z—and apply adjustable contact force to optimize coupling to the user's anatomy and maintain stable stimulation over the session. Force-feedback sensors assess the contact pressure and adjust actuator position dynamically to maintain user comfort and optimal transmission.

In certain embodiments, the system further comprises one or more robotic actuation assemblies configured to position, orient, and stabilize the stimulation components relative to the user's craniofacial anatomy. Each actuator may include a multi-degree-of-freedom positioning arm incorporating servo motors, harmonic drives, or compliant joints that enable fine spatial alignment of the stimulation assembly with the user's head or neck contour. The actuators may include force-sensing elements or tactile feedback sensors integrated within contact pads or joints, allowing the controller to apply and maintain a calibrated contact pressure against the craniofacial surface. Real-time position feedback from encoders, strain gauges, or pressure sensors is processed by the system's control logic to ensure consistent coupling and to prevent excessive mechanical loading. The robotic subsystem may operate autonomously under closed-loop control or cooperatively under user or clinician guidance.

In another embodiment, the stimulation assembly incorporates transcranial alternating-current stimulation (tACS) electrodes positioned to deliver controlled alternating electrical fields to cortical regions underlying the cranial or cervical surface. The electrodes may be integrated into the craniofacial bar or topper structure, the headband, or adhesive pads placed along the mastoid, frontal, or temporal areas. The system's controller synchronizes the phase and frequency of the tACS waveform with the vibroacoustic and photonic outputs to achieve multi-modal phase coherence and enhanced neuromodulatory efficacy. Stimulation current amplitude, waveform symmetry, and duty cycle are dynamically adjusted by the adaptive control engine based on real-time EEG feedback and historical response data. The integrated tACS pathway enables modulation of cortical oscillations, particularly within the alpha, beta, or gamma bands, while maintaining user comfort and safety through closed-loop impedance and thermal monitoring.

### Neural-Signal Acquisition Unit (HD-EEG)

The system includes a high-density EEG sensor array configured to monitor the user's brain activity with spatial resolution across cortical regions. The electrode cap or sensor array may comprise up to hundreds of channels (e.g., 128, 256 or more) distributed over the scalp, including frontal, temporal, parietal and occipital sites. The EEG array records neural oscillations in standard frequency bands including delta (0.5-4 Hz), theta (4-8 Hz), alpha (8-13 Hz), beta (13-30 Hz) and gamma (>30 Hz). The recorded EEG data are processed by the computing system to extract features such as dominant peak frequencies, phase coherence, latency shifts, brain-body entrainment markers and response to prior stimulation. The acquisition unit is operatively coupled to the computing system for real-time streaming and closed-loop control.

### Computing System and Personalization Algorithm

The computing system comprises one or more processors, memory and appropriate software modules executing a personalization algorithm. The algorithm learns from the acquired EEG data and user responses—such as comfort levels, biometrics (heart rate, GSR), and session outcomes—to adjust stimulation parameters over time. The algorithm may employ reinforcement learning, Bayesian optimization, or other machine-learning techniques to determine optimal combinations of vibratory amplitude, photonic pulse timing, tACS phase-locking, contact force, and spatial orientation. The personalization algorithm continuously refines the stimulation protocol for each user.

### Control Interface and Closed-Loop Modulation

A user or practitioner-facing control interface provides session configuration, monitoring of real-time neural and stimulation parameters, feedback visualization and manual overrides. The interface allows selection of stimulation mode, target frequency bands, training protocols, and outcome tracking over time. In closed-loop operation, the system automatically adjusts stimulation outputs based on EEG feedback: for example if the user's gamma coherence falls below a threshold, the algorithm may increase vibroacoustic amplitude or advance photonic pulse phase to enhance entrainment. Similarly, if latency mismatches or discomfort are detected, the system may reduce tACS amplitude or shift phase alignment.

### Particular Embodiments Supporting Dependent Features

Vibroacoustic Layer Frequency: In embodiments, the vibroacoustic transducer is configured to vibrate in a selectable range from about 20 Hz to about 300 Hz, which has been found to efficiently couple to craniofacial bone conduction and deliver tactile resonance to cranial tissues.

Photonic Layer Wavelength and Synchronization: The photonic emission layer produces modulated light pulses in the 400-900 nm range. The modulation may involve pulse width modulation, intensity variation or frequency-modulation in synchrony with the vibratory pulses (e.g., light pulses at 40 Hz in phase with 40 Hz vibration).

tACS Subsystem Phase-Locking: The tACS subsystem applies alternate-current stimulation at 1-40 Hz. The system determines the user's endogenous oscillatory phase via EEG and aligns the tACS pulses accordingly (e.g., delivering positive current at the peak of the alpha wave).

Robotic Arm with 6-DOF and Force-Feedback: The positioning arm provides six degrees of freedom and includes force sensors to monitor contact interface pressure. In practice, the actuator applies contact force in the range of 0.1 N to 10 N depending on anatomical target, with automatic adjustment to maintain consistent coupling during session movement or posture change.

Personalization Algorithm using RL or Bayesian Optimization: The personalization module may implement a reinforcement-learning framework in which stimulation parameter combinations are treated as actions, user neural responses (e.g., increased coherence, reduced latency) as rewards, and the system converges to optimal settings over successive sessions. Alternatively or additionally, a Bayesian optimization engine explores parameter space and updates a probabilistic model of user responsiveness.

### Method of Use

Prior to a therapy session, the system performs calibration: the bone-conduction bar is positioned on the user's selected cranial surface, the robotic arm adjusts contact force and orientation, and coupling sensors verify impedance and vibratory transmission. The HD-EEG cap is affixed and baseline brain activity is recorded. The personalization algorithm computes current user state metrics (e.g., dominant gamma peak at 43 Hz, alpha coherence low, signal latency 45 ms). A session protocol is selected (e.g., cognitive focus mode) and the multi-layer stimulation assembly is configured accordingly (vibration at 43 Hz, light flicker at 43 Hz, tACS at 43 Hz in-phase). During the session, the system monitors EEG in real time; if gamma coherence remains low after 2 minutes, the algorithm increases vibration amplitude by 10% and advances photonic pulse by +2 ms to align better with endogenous phase. At session end, data (EEG response, user comfort, biometrics) is stored and used to further refine the personalization algorithm.

### Advantages and Flexibility

By combining vibroacoustic, photonic, and tACS stimulation in a mechanically stabilized cranio-facial interface controlled by robotic positioning and governed by a personalization algorithm, the system offers high precision neuromodulation with enhanced coupling, repeatability and user-specific adaptation. The closed-loop integration ensures stimuli remain synchronized with neural activity and evolve over time to improve efficacy and comfort.

### ALTERNATIVE AND ADDITIONAL EMBODIMENTS

In further embodiments, the craniofacial bar may be configured as a mandibular splint or headband structure. The vibroacoustic layer may be embedded in a pillow or cushion for seated therapy. The photonic layer may use near-infrared wavelengths above 900 nm or visible wavelengths below 400 nm without departing from the innovations. The tACS subsystem may be replaced by other transcranial electrical modalities (e.g., tDCS) as appropriate. The personalization algorithm may incorporate other biometric inputs (e.g., HRV, skin temperature) or support multi-user group synchronization. The robotic actuator may be teleoperated or pre-programmed for zero human intervention.

### Multi-Layer Stimulation Apparatus

The present disclosure relates to a neuromodulation device configured as a multi-layer stimulation apparatus comprising concentric or vertically stacked functional layers, each designed to deliver distinct forms of stimulation—vibroacoustic, photonic, and optionally electrical—harmonically coupled to produce composite neurosensory entrainment.

In one embodiment, the neuromodulation device comprises a multilayer structure incorporating:
A vibroacoustic transducer layer configured to generate mechanical oscillations in a frequency range suitable for somatosensory and craniofacial conduction;
A photonic emitter layer positioned concentrically or co-planar with the vibroacoustic layer, configured to emit light pulses whose temporal characteristics are synchronized with the vibrational output; and
An optional electrode array for transcranial or transcutaneous alternating-current stimulation, embedded within or adjacent to the layered structure.

The outputs of these layers are harmonically coupled, such that the temporal, frequency, and phase characteristics of each modality are interdependent and synchronized, resulting in a composite multisensory signal. This composite signal is designed to enhance entrainment of targeted neural oscillatory patterns by stimulating multiple sensory pathways in synchrony.

### Vibroacoustic Transducer Layer

The vibroacoustic layer comprises one or more transducer elements (e.g., piezoelectric, electromagnetic, or magnetostrictive) configured to produce mechanical oscillations across a frequency range of approximately 20 Hz to 300 Hz. These oscillations are transmitted into the user's craniofacial or mandibular tissues, enabling bone-conduction-based somatosensory stimulation. The transducer may be embedded within a flexible substrate to conform to anatomical surfaces such as the palate, jaw, temple, or mastoid region.

The vibrational amplitude and frequency may be modulated by a control signal derived from a neural feedback loop or pre-defined entrainment protocol. The transducer is mechanically coupled to adjacent layers through an elastomeric interface, as described below.

### Photonic Emitter Layer

Positioned adjacent to or within the same structural assembly as the vibroacoustic layer, the photonic emitter layer comprises one or more light-emitting elements (e.g., LEDs, laser diodes) capable of emitting modulated light in the visible and/or near-infrared spectrum (typically 400-900 nm). The photonic emission is synchronized with the vibratory output, either by:
Pulsing the light in phase with vibrational cycles;
Modulating light intensity or pulse width according to detected vibration amplitude or frequency;
Adjusting emission parameters based on composite harmonic targets derived from EEG-guided neuromodulation profiles.

The harmonic coupling of light and vibration stimuli is achieved by temporal synchronization—such that, for example, each peak of the vibratory waveform corresponds to a light pulse maximum—thus maximizing the integration of multisensory inputs by the central nervous system.

In certain embodiments, the photonic emission pattern is directly modulated by real-time vibration amplitude or frequency, such that increases in vibration intensity result in increased light pulse brightness or frequency, providing cross-modal coherence and enhancing entrainment effects.

### Optional Electrode Array for Alternating-Current Stimulation

In further embodiments, the device includes a thin-film or flexible electrode array configured for delivering alternating current stimulation (e.g., tACS). These electrodes may be embedded within the outer or inner surfaces of the multi-layer structure, positioned to interface with the skin or mucosal surfaces. The tACS output is configured to operate within 1-40 Hz and is synchronized with the vibroacoustic and photonic outputs. Phase-locked delivery ensures that electrical stimulation occurs in alignment with the vibratory and visual stimuli, facilitating convergent neural entrainment.

The electrodes may be fabricated using conductive polymers, metallic films, or bio-compatible conductive inks, depending on application site and mechanical flexibility requirements.

### Elastomeric Coupling Medium

To facilitate efficient energy transfer and minimize impedance mismatch between layers, the vibroacoustic and photonic layers are physically integrated via an elastomeric coupling medium. This medium (e.g., silicone gel, thermoplastic elastomer) provides mechanical compliance, enhances vibratory transmission across interfaces, and supports optical transparency for photonic emission.

This elastomeric material may also serve as the primary structural matrix for the entire device, encapsulating the various functional components and conforming to complex anatomical geometries. The elastomer may be doped or molded with embedded alignment structures to preserve inter-layer positioning and stability during dynamic operation.

### Dental and Craniofacial Wearable Form Factors

In certain embodiments, the multi-layer stimulation device is fabricated as a craniofacial or dental wearable assembly. Example configurations include:
A mandibular splint or retainer-style device worn intraorally;
A cranial band or facial mask incorporating the layered transducer-photonic-electrode architecture;
A temple-mounted wearable with bone-conductive contact pads, photonic emission directed toward the orbital or temporal region, and embedded electrodes for tACS.

These form factors allow for discreet, ergonomic application and stable positioning of the stimulation components against relevant anatomical structures (e.g., cranial sutures, mandible, maxilla, zygomatic arch), enhancing both efficacy and user comfort.

### Microcontroller-Based Phase Alignment

To coordinate operation across modalities, the device includes an embedded microcontroller, digital signal processor (DSP), or equivalent timing control circuit configured to:
Receive session parameters or real-time EEG-derived phase information;
Generate synchronized control signals for vibroacoustic, photonic, and electrical outputs;
Monitor timing delays and apply compensation algorithms to preserve phase alignment across channels.

This timing control unit ensures that each sensory modality is not only delivered at the intended frequency but also phase-aligned to enhance convergent stimulation and neural entrainment. In embodiments, phase offsets can be selectively introduced to explore differential modulation or phase-shifted desynchronization strategies.

### Method of Use

In operation, the device is affixed to a user in a predetermined configuration (e.g., facial, mandibular, or cranial). Upon initiation, the vibroacoustic transducer layer begins oscillation at a selected frequency (e.g., 40 Hz), with the photonic emitter layer pulsing light at the same or harmonically related frequency. If equipped, the electrode array delivers alternating current at a phase-matched frequency. The user receives simultaneous somatosensory, visual, and optionally electrical stimuli that are harmonically structured to influence targeted neural circuits.

A control module may adjust these parameters dynamically based on user feedback, biometric sensing, or EEG-derived optimization routines. The system may be operated as a standalone wearable or integrated into a larger neuromodulation ecosystem with cloud-based analytics and personalization algorithms.

### Craniofacial Bone-Conduction Bar

The present disclosure relates to a craniofacial bone-conduction neuromodulation interface comprising a bar or frame configured for mechanical coupling to anatomical structures such as the maxilla, mandible, zygomatic arch or mastoid region, at least one transducer generating vibratory or photonic stimuli transmitted through craniofacial bone, and operable connection to an adaptive control system.

### Craniofacial Bar or Frame

In one embodiment, the interface includes a rigid or semi-rigid bar or frame designed to conform to the user's craniofacial anatomy and mechanically couple to a selected site—e.g., the mandible (jaw bone), maxilla (upper jaw), zygomatic arch (cheekbone), or mastoid region (behind the ear). The bar may include adjustable length, curvature, or clamp mechanisms to accommodate anatomical variation and ensure stable contact. The mechanical coupling may be effected via pads, pads with compliant elastomeric interface, ear-hooks, mandibular splints, or custom-fitted frames that engage bone surfaces with minimal soft-tissue impedance.

### Transducer Assembly

The interface incorporates at least one transducer mounted on or integrated into the bar. The transducer may be configured to deliver either:
Vibroacoustic stimuli, in which mechanical vibration (typically in a frequency range selected for bone conduction) is delivered via the bar into the craniofacial bone structure; or
Photonic stimuli, in which light pulses or continuous photonic emissions are transmitted into or along craniofacial bone via embedded optical interfaces.

The transducer may be mounted at the contact interface between the bar and bone region, or may transmit via coupling elements (such as elastomeric pads) ensuring efficient vibration or photonic energy transfer into the bone.

### Mechanical Coupling and Energy Transmission

To maximize energy transfer and minimize impedance mismatch between the bar, the transducer, and the craniofacial bone, the device may incorporate compliant pads, acoustic coupling gels, or elastomeric interface layers. These coupling media serve to transfer vibration from the transducer to bone with minimal loss and ensure stable contact during user movement. Photonic energy may be delivered via optical fibers embedded in the bar, which direct light pulses or near-infrared emissions into bone or adjacent tissue.

### Adaptive Control System Interface

The craniofacial bar is operably connected to an adaptive control system that receives data from neural-signal acquisition modules (for example high-density EEG), biometric sensors, or user-feedback inputs. The control system adjusts parameters such as vibration amplitude, frequency, photonic pulse timing, and phase alignment based on measured neural responses. Through closed-loop modulation, the interface delivers stimuli tailored to the user's physiological state and neuromodulatory goals.

### DEPENDENT EMBODIMENTS

Dental Operatory Appliance Integration: In a further embodiment, the bar is integrated into a dental operatory appliance such as a mouthguard, bite bar, intraoral arch or mandibular splint. The device may be fabricated from dental-grade materials and fitted to the user's dental anatomy, enabling discreet intraoral placement with stable bone coupling.

Optical Fiber Photonic Transmission: In some embodiments, the interface includes embedded optical fibers routed along the bar or frame to transmit photonic energy along craniofacial structures. These fibers may deliver near-infrared or visible light pulses, directing photonic energy into bone or peri-bone tissue for neuromodulatory effect.

Integrated tACS Electrodes: In still further embodiments, the device includes integrated electrodes on the bar that permit simultaneous transcranial alternating current stimulation (tACS). The electrodes interface with the skin or mucosal surface adjacent the craniofacial bone coupling region, and deliver phase-locked current pulses in coordination with vibration and photonic stimuli to achieve composite neuromodulation.

### Method of Use

In use, the interface is positioned on the selected craniofacial bone region and secured via the bar or frame. The transducer is activated to deliver vibration and/or photonic pulses. The control system monitors the user's neural activity or biometric feedback and dynamically adjusts stimulus parameters. If integrated, tACS electrodes deliver current pulses synchronised with the transducer output. Over time, the adaptive system updates stimulation protocols to optimize efficacy and user comfort.

### Advantages and Flexibility

This craniofacial bone-conduction interface offers enhanced coupling of neuromodulatory stimuli to the skeletal system, enabling precise delivery of vibroacoustic or photonic signals directly into bone conduction pathways. Integration with dental or craniofacial appliances increases wearability and user comfort. The addition of tACS electrodes further expands the neuromodulation capability. Operable connection to an adaptive control system supports personalized, closed-loop therapy that responds to individual neural states.

### ALTERNATIVE AND ADDITIONAL EMBODIMENTS

Variations may include a bar formed of carbon-fiber or titanium for light weight and rigidity; transducers configured as miniature voice-coil actuators; optical fibers delivering pulsed light in wavelengths outside 400-900 nm (e.g., near-infrared >900 nm); electrodes for other forms of stimulation such as tDCS; a modular bar configured to slide or articulate across anatomical zones; and wireless control and power modules integrated into the bar.

### Robotic Delivery Platform

In accordance with various embodiments, the system comprises a robotic delivery platform configured to position and modulate the contact of a multi-modal neuromodulation device relative to a user's craniofacial anatomy. The robotic delivery system includes a robotic arm with at least five degrees of freedom, integrated force and position sensors, and a control algorithm that dynamically adjusts placement and stimulation parameters based on real-time neural feedback (e.g., high-density EEG). This architecture enables precise, repeatable, and adaptive coupling of neuromodulation hardware to the user's anatomical surfaces.

### Robotic Arm Structure and Degrees of Freedom

The robotic delivery system includes a robotic arm assembly configured to move and orient the neuromodulation device—such as a craniofacial bone-conduction bar, stimulation hood, or facial interface—in at least five degrees of freedom. These degrees of freedom typically include:
Three translational axes (X, Y, Z) enabling positioning in space;
Two rotational axes (pitch and yaw) enabling angular orientation of the device relative to craniofacial surfaces. In some embodiments, an additional roll axis is provided, thereby offering six degrees of freedom. The robot includes joints, actuators (e.g., servo motors, linear actuators), and is mounted to a base structure such as a clinical chair headrest, dental operatory fixture, ceiling pendulum mount or floor stand.

### Force and Position Sensing and Feedback

The robotic arm incorporates integrated force sensors and position sensors. Force sensors (such as strain gauges or load cells) monitor contact pressure between the neuromodulation device and the craniofacial anatomy, ensuring safe and optimal coupling. Position sensors (such as encoders, potentiometers or optical trackers) record the precise spatial coordinates and orientation of the device relative to the user's anatomy. The feedback from these sensors is used by the control algorithm to maintain stable contact, adjust contact force dynamically, compensate for user movement, and optimize energy transmission (e.g., vibroacoustic coupling, photonic delivery, electrode contact).

### Neural-Feedback Controlled Placement

A control algorithm is executed by the computing system to dynamically adjust the stimulation placement and orientation based on real-time neural feedback. High-density EEG signals collected from the user are processed to extract metrics such as oscillatory power, coherence, phase alignment, latency, and neural response to current stimulation. When neural feedback indicates sub-optimal engagement (e.g., low gamma coherence, latency drift, under-resonance of vibroacoustic delivery), the control algorithm issues commands to the robotic arm to reposition the device (adjusting translation, orientation or contact force) or to modify stimulation parameters (e.g., vibration amplitude, photonic pulse timing, tACS electrode contact). This closed-loop adjustment ensures that stimulation remains aligned with the user's anatomy, physiology, and neural state throughout the session.

### Calibration and Automated Resonance Mode Optimization

In certain dependent embodiments, the robotic control system is adapted to automate calibration of vibroacoustic resonance modes across cranial structures. During a setup or calibration phase, the robotic arm systematically adjusts the contact location, orientation, and actuator drive of the vibroacoustic transducer layer while monitoring EEG or accelerometer feedback. The system identifies the optimal coupling geometry that maximizes resonance coupling (e.g., highest amplitude transmission, strongest neural response). Once optimal placement is identified, the robot stores the position and applies it for subsequent sessions, ensuring repeatable precision.

### Dental Operatory or Clinical Chair Integration

In further embodiments, the robotic delivery platform is integrated into a dental operatory or clinical chair environment. The robotic base may be affixed to the headrest of a dental chair, or to ancillary equipment used in clinical neuromodulation settings. The user is seated or semi-supine, and the robot positions the neuromodulation device relative to the maxilla, mandible, zygomatic arch or mastoid region. Integration into clinical furniture supports high-volume throughput, practitioner supervision, and ergonomic access.

### Method of Use

In a typical session, the user is seated in the clinical chair or treatment chair, wearing or positioned relative to the neuromodulation device. The robotic arm moves the device into initial placement under practitioner guidance or automated pre-programmed positioning. The force sensors verify adequate coupling pressure; the position sensors record spatial configuration. The neural-signal acquisition system records baseline EEG activity. The personalization or calibration routine is executed. If the feedback algorithm detects insufficient neural engagement, the robotic arm makes incremental adjustments to placement and orientation; stimulation parameters are adjusted as needed. The session proceeds with closed-loop monitoring and adaptive placement until completion. At session end, placement data, coupling force records, EEG response metrics and stimulation logs are stored for future optimization.

### Advantages

The robotic delivery platform enhances neuromodulation by providing:
Precise and reproducible placement of stimulation hardware relative to craniofacial anatomy;
Real-time adaptation to the user's anatomy and physiology, including repositioning in response to movement or coupling drift;
Automated calibration of resonance and coupling modes, reducing practitioner burden and improving consistency;
Integration into standard clinical and dental furniture, enabling seamless workflow in therapeutic settings.

### ALTERNATIVE EMBODIMENTS

Variations may include robotic arms with different mounting configurations (ceiling-mounted, floor stand, mobile cart), replacement of force sensors with tactile sensors or optical contact sensors, integration of haptic feedback for practitioner override, and extension to multi-user robotic arrays for group therapy. The adaptive control algorithm may incorporate machine-learning methods to refine placement strategies over repeated sessions.

### EEG Mapping and Validation

In various embodiments, the disclosure provides a method for mapping and validating consumer-grade electroencephalography (EEG) signals against high-density EEG measurements. This method comprises concurrently acquiring signals from a consumer-grade EEG device and a high-density EEG array during multimodal stimulation, computing spatial and temporal correlation metrics to calibrate signal fidelity, and generating a mapping function to align the consumer EEG signal space to laboratory-grade EEG space. Dependent embodiments further include using the mapping function to retrain a machine-learning personalization model, and adapting stimulation parameters of a portable neuromodulation device in accordance with that model.

### Concurrent Acquisition of Consumer-Grade and High-Density EEG Signals

The method begins by selecting a user and preparing two EEG systems: one consumer-grade EEG device (for example a low-density wearable headband) and one high-density EEG (HD-EEG) array (for example a 64, 128, or 256-channel cap). The user is fitted simultaneously with both devices. The consumer-grade device may include fewer electrodes (e.g., 4-16 channels), dry electrodes, wireless transmission, and minimal setup, whereas the high-density array provides full scalp coverage, high sampling rate and optimized electrode contact. Barrow Neurological Institute+1

Multimodal stimulation is delivered to the user (for example vibroacoustic vibration and photonic pulsing) while both EEG systems record concurrently. The stimulation protocol may include baseline (no stimulation), active stimulation, rest periods, and tasks. By acquiring simultaneous data, the method captures temporally aligned signal streams from both systems during the identical physiological conditions.

### Computation of Spatial and Temporal Correlation Metrics

Following data acquisition, the signals from both EEG systems are processed to align in time and space. Pre-processing may include artifact removal, band-pass filtering, referencing, and normalization. Temporal correlation metrics such as cross-correlation coefficients between corresponding frequency-band power time-series (e.g., alpha, beta, gamma) are computed. Spatial correlation metrics are computed by comparing topographic distributions from the high-density array with the reduced spatial sampling of the consumer device-mapping e.g., the consumer channel amplitudes onto the HD-EEG scalp map and computing similarity indices. For example, a study found that the correlation of power spectral density between a low-density headband and a lab system in the alpha band reached r≈0.87. Frontiers+1

From these analyses, calibration coefficients or error-maps are derived reflecting the systematic differences in amplitude scaling, latency shifts, spatial sampling bias or frequency sensitivity between the two devices. Latency differentials (e.g., consumer system delayed by X ms relative to HD-EEG) and amplitude scale factors (e.g., consumer device underestimates gamma power by Y %) are logged.

### Generation of a Mapping Function

Using the calibration coefficients and error-maps, the method generates a mapping function that transforms the signal space of the consumer-grade EEG device into a virtual representation aligned with the high-density EEG space. The mapping function may include linear regression transformations, polynomial adjustments, spatial interpolation, latency compensation filters, or machine-learning-based transformations (e.g., a neural network model). The function calibrates for amplitude scaling, latency offsets, electrode montage differences, and spatial undersampling artefacts.

Once established, this mapping function enables the portable consumer device to yield derived outputs that approximate the fidelity and neurophysiological correspondence of the HD-EEG system, thereby enabling downstream personalization algorithms to operate on normative data frameworks.

### Use of Mapping Function to Retrain Personalization Model

In a dependent embodiment, the mapping function is employed to retrain a machine-learning personalization model that controls multi-modal stimulation parameters. The personalization model (for example a reinforcement-learning agent or Bayesian optimizer) receives input from the consumer-grade EEG device. Because the mapping function transforms the raw consumer signals into a mapped “high-density equivalent” space, the trained model can leverage higher-resolution representations of neural state. This supports improved decision-making in the personalization algorithm.

### Adaptation of Stimulation Parameters of Portable Neuromodulation Device

In a further dependent embodiment, the system adapts stimulation parameters of a portable neuromodulation device based on the calibrated consumer-grade EEG input. The mapping function provides adjusted real-time neural metrics (e.g., corrected gamma coherence, phase locking value) which feed into the personalization algorithm. The algorithm dynamically adjusts vibroacoustic amplitude, photonic pulse rate, tACS phase or electrode configuration accordingly. Because the consumer device now approximates lab-grade fidelity, the portable device can deliver high-precision neuromodulation outside of laboratory settings.

### Method Flow Summary

Fit user with consumer-grade EEG device and HD-EEG array simultaneously.

Deliver multimodal stimulation and recording protocols, capturing synchronous signal sets.

Pre-process both signal sets, compute temporal and spatial correlation metrics, derive calibration coefficients.

Build a mapping function transforming consumer data into HD-EEG aligned data space.

Use mapping function to retrain personalization model for stimulation control.

Deploy portable neuromodulation device using consumer EEG input, applying mapped metrics to dynamically adapt stimulation parameters.

Over subsequent sessions, refine the mapping function and personalization model based on new data (e.g., changes in user neural response, electrode drift, adaptation).

### Advantages

This method enables the use of low-cost, consumer-grade EEG devices in high-precision neuromodulation systems by bridging the fidelity gap to laboratory-grade HD-EEG. It allows scalable personalization of stimulation protocols beyond the laboratory, supports longitudinal tracking of neural state, and enhances accessibility of neuromodulation therapy. By calibrating and aligning consumer EEG signals with high-density systems, the system improves signal reliability and enables portable devices to deliver clinically meaningful neuromodulation.

### ALTERNATIVE EMBODIMENTS

In further embodiments, the mapping function may be updated continuously with cloud-based aggregated data across user populations to improve accuracy (transfer learning). The consumer device may include onboard preprocessing to compute latency differentials and auto-adjust electrode contact. The mapping may incorporate biometric covariates (e.g., head size, electrode impedance, scalp thickness) to refine transformations. The personalization model may incorporate real-time feedback loops for stimulation efficacy, and the portable device may adapt stimulation in real time based on the mapped metrics.

### Grid Personalization Engine

In various embodiments, the disclosure provides a personalization engine (referred to herein as the “Grid”) comprising a neural-data repository, a machine-learning model configured to predict optimal stimulation parameters for individual users, and an adaptive feedback loop that updates model weights based on longitudinal outcomes. The Grid supports enhanced personalization via aggregation of multi-user data, transfer learning for new users, and generation of digital-twin models of user neuro-sensory response patterns.

### Neural Data Repository

The Grid includes a neural data repository designed to store user-specific EEG data and behavioral or response data gathered over time. EEG data may include high-density EEG recordings (e.g., 64-channel, 128-channel), user-specific neuromaps, latency/phase response measures, entrainment metrics, and stimulation protocol metadata (frequency, amplitude, phase offsets, modality combinations). Behavioral response data may include user comfort ratings, cognitive test performance (e.g., reaction time, memory accuracy), physiological biomarkers (heart rate variability, galvanic skin response), session outcomes (entrainment achieved %, adherence, side-effects) and calibration logs. The repository supports longitudinal storage for each user and retains versioned data to track progression over repeated sessions.

### Machine-Learning Model for Prediction of Stimulation Parameters

An adaptive machine-learning module is configured to receive inputs from the repository and output predicted stimulation parameters for individual users. The model may be implemented using reinforcement-learning algorithms (e.g., Q-learning, policy gradient), Bayesian optimization frameworks, deep neural networks, ensemble methods or a hybrid architecture. Inputs to the model include user-specific features (baseline neural signature, anatomical metrics, prior response history), cross-user metadata (age, head size, impedance history) and contextual session variables (time of day, posture, modality configuration). Outputs include recommended stimulation modalities (vibroacoustic frequency, photonic pulse rate, tACS phase), spatial delivery parameters (transducer placement, contact force), and timing/latency offsets.

### Adaptive Feedback Loop

The Grid implements a closed-loop adaptive feedback mechanism in which model weights are updated over time based on longitudinal outcomes. After each therapeutic session, the system records outcome metrics (e.g., change in EEG coherence, entrainment efficiency, user comfort, biometric response). The feedback loop uses these outcomes to adjust the machine-learning model: for example, increasing weight on input features that correlate with positive response and reducing weight on features that yield poor outcomes. The loop may include supervised updating (where outcome labels are assigned), unsupervised clustering of user response trajectories, and meta-learning to accelerate adaptation across users.

### Aggregation of Multi-User Data

In embodiments corresponding to dependent claims, the Grid aggregates anonymized data across multiple users stored in the repository to enhance personalization algorithms. The multi-user dataset may include demographic metadata (age, sex, head circumference, prior neuromodulation history), anatomical metrics, neural signatures and response outcomes. The dataset is used to extract population-level patterns and clusters of user types (responders vs non-responders), enabling improved prediction accuracy and generalization. For example, clustering may identify a user subgroup with a specific gamma peak at ~43 Hz who responds optimally to vibroacoustic+photonic entrainment at 43 Hz, thereby allowing the model to propose parameters for new users with similar profile.

### Transfer Learning for New Users

The Grid supports transfer-learning functionality to enable new users to receive initial stimulation parameters based on clustered similarity to existing users. When a new user enters the system, baseline neural and anatomical data are collected; the model references the aggregated multi-user dataset to find the most similar cluster(s) of prior users. Initial stimulation parameters are then provisioned according to the predictions derived for that cluster, thereby reducing calibration time and improving early session efficacy. As the new user accumulates session outcome data, the adaptive feedback loop fine-tunes model weights specific to the individual.

### Digital Twin Generation of User Neurosensory Response Patterns

In further embodiments, the engine generates a digital-twin model of each user's neurosensory response patterns. The digital twin is a simulated representation of the user's brain-body dynamics, including mapped neural oscillatory behaviour, anatomical coupling characteristics, vibrational transfer functions and expected stimulation responses. The twin model is updated over time with empirical data, allowing “what-if” simulation of stimulation parameter changes and prediction of entrainment outcomes before live delivery. The digital twin supports personalized visualization, session planning, safety simulation and protocol optimisation.

### Method Flow

Neural and response data are ingested into the repository from each session (EEG, biometrics, stimulation logs).

The machine-learning model processes input features and outputs recommended stimulation parameters for the next session.

The personalization engine applies the recommended parameters via the neuromodulation device.

After the session, outcome metrics are collected and fed back into the adaptive feedback loop.

Model weights are updated and the user's digital twin is refined accordingly.

Over time across users, multi-user aggregation informs cluster formation, transfer-learning provisioning for new users, and continuous improvement of the model.

### Advantages

Enables truly individualized neuromodulation by leveraging both personal and population-level data.

Reduces calibration time and improves early-session efficacy for new users via transfer-learning.

Supports simulation (digital twin) to anticipate and optimise stimulation before use.

Facilitates continual improvement across user base through aggregate analytics and adaptive model updating.

Enhances safety and consistency by using longitudinal outcome tracking and model-driven parameter adjustment.

### ALTERNATIVE AND ADDITIONAL EMBODIMENTS

The personalization engine may be implemented in cloud-based infrastructure enabling distributed machine-learning across user populations, with federated learning for data privacy. Model types may include graph neural networks capturing user-session network dynamics. The digital twin may incorporate biomechanical and anatomical finite-element models of craniofacial coupling. Stimulation parameter prediction may include additional modalities (olfactory, thermal, haptic) in future expansions. Data streams may also include environmental sensors (temperature, ambient noise) or lifestyle factors (sleep quality, circadian phase) to further refine personalization.

In various embodiments, the disclosure provides a dental operatory system that incorporates a neuromodulation apparatus (as defined in preceding claims) mounted on or integrated with a dental chair. The system comprises a patient headrest containing vibroacoustic and photonic transducers, a robotic positioning arm for craniofacial targeting, and integration with operatory controls for synchronization with dental procedures. Dependent embodiments include features such as reduction of procedural anxiety or modulation of pain perception via entrainment, and electronic interfacing of dental instruments with the neuromodulation control unit.

Patient Headrest with Vibroacoustic & Photonic Transducers

The dental operatory system includes a headrest assembly attached to the dental chair. The headrest is configured to support the patient's head in treatment posture and integrates:
A vibroacoustic transducer layer, embedded within or behind the headrest cushion, designed to deliver mechanical vibrations (e.g., 20-300 Hz) via cranial contact surfaces (such as the mastoid or zygomatic arch).
A photonic emitter layer, co-located with or adjacent to the vibroacoustic layer, capable of emitting modulated light (e.g., 400-900 nm) synchronized with vibration output.
The headrest maintains comfortable ergonomic support while simultaneously coupling neurosensory stimulation to the patient, facilitating entrainment, anxiety reduction, or pain modulation during dental procedures.

### Robotic Positioning Arm for Craniofacial Targeting

A robotic positioning arm is mounted to the dental chair infrastructure (for example at the headrest frame or chair base) and is configured to precisely position the neuromodulation device relative to the patient's craniofacial anatomy. The arm provides multiple degrees of freedom to adjust orientation, translation and contact force of the headrest/stimulation assembly. Force and position sensors ensure appropriate coupling pressure, and the arm may reposition during treatment if the patient's head moves or the dentist shifts chair position. This robotic control ensures consistent stimulation placement and optimal coupling throughout the dental session.

Integration with Operatory Controls

The neuromodulation system is fully integrated with the dental operatory controls. The interface includes:
An operatory control console or software module that links chair movement, headrest position, drill/handpiece status and session timing with stimulation protocols.
The neuromodulation control unit communicates with dental instruments (e.g., high-speed handpieces, ultrasonic scalers, suction systems) to synchronize sensory stimuli with procedural events. For instance, when the drill activates, the system may initiate a vibration-photonic entrainment sequence to reduce patient discomfort or anxiety.
The headrest stimulation is timed in coordination with dental treatment workflow, such that neuromodulatory input supports the procedural context (e.g., injection, drilling, cleaning) without interfering with operator access or visibility.

### Method of Use

In a typical dental operatory session, the patient is seated in the chair and reclined as required. The headrest with integrated transducers is positioned and coupled via the robotic arm to the patient's craniofacial region. The neuromodulation control unit is activated and linked to the operatory controls. During treatment, the system delivers synchronized vibroacoustic and photonic stimulation aligned with key procedural events (e.g., anesthesia injection, restorative drilling). The robot monitors and maintains optimal contact; the operatory-neuromodulation system tracks instrument use and timing to trigger or adjust stimulation. Upon completion, stimulation ceases, and data may be logged for session outcome monitoring.

### Benefits: Anxiety and Pain Modulation

In one embodiment, the system is configured to reduce procedural anxiety or modulate pain perception via entrainment. The vibroacoustic and photonic stimuli can be tailored (for example to theta/alpha bands) to promote calming neural states and parasympathetic activation. The synchronous stimulation during injection or drilling may reduce perceived pain intensity, lower physiological stress markers (heart rate, GSR) and improve patient comfort, thereby enhancing compliance and reducing agitation.

### Instrument-Control Interface

In further embodiments, dental instruments interface electronically with the neuromodulation control unit. For example:
The handpiece activation signal (on/off) may trigger a specific stimulation pattern (e.g., increased vibration amplitude during drilling onset).
The suction or irrigation system may send status signals to the control unit to modulate stimulus timing (e.g., pause entrainment during cooling spray, resume during quiet phases).
The system logs instrument events, stimulation timestamps and biometric responses for subsequent analysis and personalization of future sessions.

### ALTERNATIVE AND ADDITIONAL EMBODIMENTS

The headrest may be removable and converted to a mobile neuromodulation cushion for home use. The robotic arm may integrate haptic feedback for dentist awareness of coupling force. The photonic layer may include near-infrared LEDs above 900 nm for deeper tissue penetration. Stimulation may be extended to include auditory or olfactory modules integrated into the operatory environment. The system may use real-time EEG monitoring from the patient (or a cluster of patients) to adapt stimulation mid-procedure in closed-loop fashion.

### Therapeutic and Adaptive Control

In various embodiments, the disclosure provides a method for adaptive neuromodulation comprising: acquisition of baseline EEG data; application of multi-layer stimulation including vibroacoustic, photonic, and electrical modalities; monitoring of real-time EEG response; adjustment of stimulation parameters via an adaptive control algorithm; and iterative refinement of user-specific neuromodulation profiles over time. Dependent embodiments include methods whereby symptom reduction (stress, anxiety, chronic pain) is achieved and wherein parameter updates are transmitted via a cloud-based personalization engine (“Grid”) to remote devices.

### Step 1: Acquisition of Baseline EEG Data

The method begins by placing a user in a neuromodulation environment and affixing an EEG sensor array—preferably a high-density EEG (HD-EEG) cap or equivalent sensor system. Baseline neural activity is recorded over a defined period (e.g., 2-10 minutes) during a resting state or standardized task. The baseline data capture endogenous oscillatory power distributions (e.g., delta, theta, alpha, beta, gamma), phase relationships, latency metrics, and brain-body coupling characteristics. These baseline metrics establish the initial neural profile for the user and serve as input to the personalization algorithm and subsequent stimulation planning.

### Step 2: Application of Multi-Layer Stimulation

Following baseline acquisition, the method applies a multi-layer stimulation sequence comprising:
Vibroacoustic stimulation delivered via transducer elements (e.g., 20-300 Hz) coupled to craniofacial or body surfaces;
Photonic stimulation delivered via modulated light emissions (e.g., 400-900 nm), temporally synchronized with vibroacoustic output; and
Electrical stimulation (optional) delivered via transcranial alternating current (tACS) or other modalities (e.g., 1-40 Hz), phase-locked to target EEG oscillations.

The stimulation is initiated according to a protocol derived from the user's baseline neuromap, or pre-configured for therapeutic goals (e.g., focus enhancement, emotional regulation, pain modulation).

### Step 3: Monitoring Real-Time EEG Response

While the multi-layer stimulation is delivered, the system continuously monitors the user's EEG in real time. Neural responses are processed to extract metrics such as oscillatory amplitude change, phase-locking value (PLV), coherence among brain regions, latency shifts, and entrainment magnitude. The system also monitors ancillary biometric signals (e.g., heart rate variability, galvanic skin response) as adjunctive indicators of physiological engagement.

### Step 4: Adjustment of Stimulation Parameters Via Adaptive Control Algorithm

Based on the real-time EEG (and optionally biometric) feedback, the adaptive control algorithm dynamically adjusts stimulation parameters. These adjustments may include:
Changing vibroacoustic amplitude, frequency or waveform shape;
Modulating photonic pulse timing, intensity or wavelength;
Altering tACS amplitude, phase alignment, or frequency;
Modifying spatial placement or contact force of transducers;
Sequencing stimuli to maintain phase alignment and latency compensation.

The algorithm may employ machine learning, reinforcement learning, or Bayesian optimization to tune parameters so as to maximise EEG entrainment, neural coherence, or therapeutic effect while maintaining user comfort and safety. The closed-loop adjustment continues during the session to respond to evolving neural state and coupling conditions.

### Step 5: Iterative Refinement of User-Specific Neuromodulation Profiles Over Time

After each session, outcome data (EEG response metrics, user-reported outcomes, biometric data, calibration logs) are stored in the personalization repository. The method iteratively refines the user-specific profile by updating the personalization model with longitudinal data, thereby converging toward optimal stimulation parameters for that user. The neuromodulation profile includes preferred frequencies, amplitude thresholds, contact positions, phase offsets, and modality combinations. Future sessions begin with these refined parameters, reducing calibration time and improving efficacy.

### Therapeutic Implementation (Dependent Embodiments)

In one dependent embodiment, the method is applied to reduce symptoms associated with stress, anxiety, or chronic pain. For example, stimulation protocols targeting theta/alpha bands (4-10 Hz) may be used for anxiety reduction; pain-modulation protocols may combine vibroacoustic and photonic stimulation to engage somatosensory gating pathways while monitoring neural biomarkers indicative of pain relief Outcome measures include reduction in self-reported anxiety/pain scores, improvement in HRV, reduction of GSR peaks, and increased neural entrainment coherence. The iterative adaptive method facilitates sustained therapeutic benefit over multiple sessions.

### Cloud-Based Parameter Update (Dependent Embodiment)

In another dependent embodiment, the method includes transmission of stimulation parameter updates via a cloud-based personalization engine (“Grid”) to remote neuromodulation devices. The repository aggregates anonymized multi-user data, and the personalization model computes updated parameters which are then pushed to portable or wearable neuromodulation devices in the field. This cloud-based update mechanism ensures that device firmware, waveforms, entrainment schedules or default settings remain current and optimized based on aggregated learning from multiple users.

### Method Flow Summary

Fit user with EEG sensors and record baseline neural data.

Apply multi-modal stimulation (vibroacoustic+photonic+electrical).

Monitor real-time EEG response; extract entrainment and coherence metrics.

Via adaptive algorithm, adjust stimulation parameters dynamically during session.

Post-session, store outcome data, update personalization model, refine neuromodulation profile.

Over subsequent sessions, apply refined parameters; for remote devices, parameter updates may propagate via cloud-based engine.

Assess therapeutic outcomes (stress, anxiety, pain), and integrate into feedback loop to further optimize stimulation protocol.

### Advantages

The described method enables adaptive neuromodulation that is responsive to individual neural states, minimizing one-size-fits-all protocols. It allows real-time fine-tuning of multi-modal stimulation and supports longitudinal personalization. The inclusion of cloud-based updates enhances scalability, allowing portable devices to benefit from aggregated population learning and rapid deployment of optimized protocols. The method supports therapeutic outcomes across a range of indications via targeted entrainment and modulation of neural circuits.

### ALTERNATIVE AND ADDITIONAL EMBODIMENTS

Alternative embodiments may include sleep-state neuromodulation, wearable home-use devices, group therapy sessions with synchronized stimulation across participants, or multi-session digital twin modeling. The method may incorporate additional sensor inputs (e.g., respiration, motion tracking) and environmental modulation (e.g., temperature, lighting) to enrich the adaptive loop. The stimulation protocol may also include scheduled phases for entrainment, consolidation and rest.

### Machine Learning and Cloud Integration

The present disclosure includes a computer-implemented method for adaptive control of neuromodulation, utilizing machine learning algorithms trained on multimodal stimulation-response datasets. The method supports individualized prediction and continuous adaptation of stimulation parameters based on EEG-derived feedback, and synchronizes said parameters across both local devices and a cloud-based personalization infrastructure referred to as the “Grid.”

This software-based system forms the core computational intelligence of the adaptive neuromodulation platform and is applicable across multiple use cases, including therapeutic, cognitive, wellness, and clinical deployments.

### Training a Neural Network on Multimodal Stimulation-Response Datasets

In one embodiment, the system includes a training phase in which a neural network is trained using datasets comprising:
Multimodal stimulation parameters (e.g., frequency, amplitude, modality combination);
Corresponding neurophysiological responses, including high-density EEG data, biometric signals (e.g., heart rate variability, skin conductance), and behavioral feedback (e.g., pain scores, mood ratings);
Contextual metadata, such as time of day, device configuration, demographic information, and session objectives.

The training process uses supervised, semi-supervised, or reinforcement learning paradigms, with the objective of modeling the relationship between input stimulation parameters and resulting neural or behavioral outcomes.

Preferred network architectures include convolutional neural networks (CNNs), recurrent neural networks (RNNs), transformer models, or ensemble learning systems, optionally augmented by Bayesian optimization layers or attention mechanisms to prioritize features and responses most relevant to each user's state.

### Predicting User-Specific Stimulation Parameters

Following training, the neural network is configured to predict optimal stimulation parameters for individual users based on incoming data. This may include:
Baseline EEG features (e.g., power spectral density, coherence, phase lag index);
Current brain state classification (e.g., alert, fatigued, anxious, pain);
Historical session data and prior response curves.

The model outputs a set of predicted parameters including vibratory frequencies, light modulation patterns, electrical current profiles (e.g., tACS waveform), and timing schedules across modalities. The stimulation pattern may be encoded as a spectrotemporal envelope or control sequence.

These predictions are tailored to maximize user-specific objectives such as relaxation, focus, mood stabilization, or sleep enhancement.

### Continuously Updating Parameters Based on EEG-Derived Reward Signals

During live sessions, the system operates in a closed-loop fashion, using real-time EEG input to continuously assess the efficacy of the current stimulation pattern. Reward signals may include:
Increase in neural entrainment at target frequency bands;
Enhanced phase locking between brain regions;
Improvement in biometric indicators of autonomic regulation;
Subjective feedback from the user interface.

The neural network uses these signals to update model weights or apply online learning techniques, such as stochastic gradient descent or policy gradient updates. This enables ongoing refinement of the stimulation profile, even within a single session, and across sessions longitudinally.

This component is designed to maintain therapeutic alignment with dynamically changing neural states and environmental conditions.

### Synchronizing Across Local and Cloud-Based Instances of the Personalization Grid

To ensure scalability and global learning, the system includes mechanisms for synchronizing personalized models and parameters across local devices (e.g., tablets, wearables, embedded processors) and a centralized cloud-based personalization engine (“the Grid”).

This synchronization enables:
Download of model updates or stimulation templates derived from global data aggregation;
Upload of anonymized EEG and outcome data from local sessions;
Resolution of conflicts or divergences between local and cloud predictions via ensemble voting or averaging mechanisms;
Security protocols including data encryption, access control, and compliance with healthcare data regulations (e.g., HIPAA, GDPR).

The cloud-based Grid architecture supports distributed computation and storage, enabling federated learning models and transfer learning pathways. This ensures that even new users with limited initial data can benefit from patterns learned across the broader population.

### Summary of Functional Advantages

The described method:
Enables dynamic, real-time personalization of neuromodulation therapy;
Learns from large-scale datasets and adapts to individual neurophysiology;
Operates across decentralized hardware devices while leveraging centralized cloud intelligence;
Improves safety, efficacy, and user engagement through predictive and adaptive modeling;
Facilitates population-level analytics and evidence-based protocol refinement.

### Integrated Personal Device

In various embodiments, the disclosure provides a consumer neuromodulation headset or craniofacial wearable device comprising: an integrated multi-modal stimulation module (vibroacoustic, photonic, tACS), embedded EEG sensors, and wireless communication capability to a personalization engine (the “Grid”) as defined in previous claims. The device supports storage and transmission of usage metrics for longitudinal adaptation, and may include vibration-driven photonics that generate localized light fields through resonance coupling. The device is designed for consumer form-factor use—portable, comfortable, user-configurable—and enables personalized, adaptive neuromodulation outside of clinical environments.

### Integrated Multi-Modal Stimulation Module

The wearable device houses a stimulation module with concentric or stacked layers:
A vibroacoustic transducer layer, configured to deliver mechanical vibration (e.g., 20 Hz-300 Hz) via craniofacial bone conduction or wearable contact surfaces (e.g., around the zygomatic arch, mastoid, mandible).
A photonic emitter layer, comprising visible and/or near-infrared light sources (e.g., 400-900 nm) temporally synchronized to the vibroacoustic signal. The photonic emission may be modulated in amplitude, pulse width, or pattern according to stimulation protocols.
An electrical stimulation layer (tACS subsystem), optionally embedded, capable of delivering alternating current stimulation (e.g., 1-40 Hz) through electrodes integrated into the wearable frame, positioned near scalp or craniofacial surfaces.

In one embodiment, the photonic layer is vibration-driven: the vibroacoustic transducer modulates coupling into the photonic emitter via an elastomeric coupling medium, creating resonance-driven light fields that are localized to craniofacial bone and soft-tissue contacts, enhancing energy transfer and sensory convergence.

### Embedded EEG Sensors

The headset incorporates EEG sensor elements—either dry-electrodes or low-impedance gel-free contacts—that make contact with the user's scalp or skin in accordance with a wearable layout optimized for consumer comfort. These sensors continuously monitor brainwave activity across multiple frequency bands (e.g., delta, theta, alpha, beta, gamma), and support real-time neural feedback. The EEG subsystem may include signal amplification, filtering, analog-to-digital conversion, and wireless streaming or local edge-processing. Design principles from wearable EEG headsets are applicable (e.g., miniaturized dry electrodes, comfortable headband form-factor). MDPI+2Bitbrain+2

### Wireless Communication to Personalization Grid

The wearable device is equipped with a wireless communication interface (e.g., Bluetooth Low Energy, Wi-Fi, or proprietary RF) that links to the personalization engine (the Grid) described in prior claims. Usage data—including stimulation parameters, EEG response metrics, session duration, user-reported feedback—is stored locally and/or transmitted to the repository. The device receives updated stimulation profiles or firmware via the Grid, enabling cloud-based adaptation and updates across the device lifecycle.

### Usage Metrics Storage and Transmission

In dependent embodiments, the device stores longitudinal usage metrics in onboard memory or transmits them to the Grid. Metrics include: number of sessions, cumulative stimulation time, average amplitude and frequency values, neural entrainment indexes (e.g., phase-locking value, coherence), user comfort ratings, battery cycles, contact impedance trends, and maintenance logs. This data supports adaptation of personalization algorithms, trend analysis, and firmware updates to refine device response for the individual user over time.

### Vibration-Driven Photonics for Localized Light Fields

In another dependent variant, the device integrates a coupling mechanism where vibroacoustic vibration drives photonic emission via mechanical resonance. For example, the photonic emitter layer may be mounted on an elastomeric substrate attached to the transducer. When the transducer vibrates, the elastomeric coupling concentrates stress or that vibration into the photonic material, producing enhanced light modulation or directional light fields that conform to craniofacial contours. These localized light fields penetrate shallow bone or soft tissue and augment sensory input by spatially aligning optical and mechanical modalities in the same anatomical axis.

### Consumer Form-Factor Design Considerations

The headset is designed for comfort, ergonomics, aesthetics, and ease of use. Key features may include:
Adjustable headband or craniofacial strap to fit a range of head sizes;
Lightweight construction (<300 g) to enable home use;
Quick-connect modules for charging and firmware updates;
Integrated battery sufficient for multiple sessions (e.g., 2-4 hours);
User-interface via mobile app for session selection, feedback entry, and data visualization;
Safety features such as contact impedance monitoring, current and temperature limitation in tACS electrodes, and automatic shut-off if EEG response falls outside safe bounds.

### Method of Use

In a typical consumer session:
The user dons the headset, ensuring contact of transducers and EEG sensors.
The device powers on, streams baseline EEG to the Grid or local processor, and calibrates contact impedance and stimulation readiness.
The stimulation module is activated according to a personalized parameter set received from the Grid: vibroacoustic pulses, synchronized light pulses, and optional tACS currents.
EEG sensors monitor neural response in real time; data is either processed locally or streamed to the cloud.
Usage metrics and outcome feedback (e.g., user selects “focus”, “relax”, “sleep”) are uploaded to the Grid.
Over multiple sessions, the Grid refines the user-specific profile and updates parameter sets or firmware which are downloaded to the headset.

### Advantages

Enables consumer-accessible neuromodulation with integrated sensing, stimulation, and connectivity.

Supports closed-loop personalization and cloud-based adaptation for each user.

Combines multiple stimulation modalities (vibration, light, electrical) in a single wearable device for richer neurosensory impact.

Enhances user engagement and adherence by delivering therapy in a portable, comfortable head-worn form-factor.

Data accumulation supports refinement of personalization models and long-term optimization of effects.

### ALTERNATIVE EMBODIMENTS

Alternative embodiments may include modular add-on attachments (e.g., ear-cups, chin strap modules), extended electrode arrays for higher channel count EEG, integration with prosthetic or AR/VR headgear, or incorporation of additional sensors (e.g., accelerometers, GSR, PPG) for multimodal feedback. The photonic layer may include holographic patterns or wearable micro-LED arrays. Firmware may support over-the-air updates and integration with smartphone apps for gamified neuromodulation or wellness routines.

In some embodiments, the disclosed adaptive neuromodulation system includes a multi-layer stimulation assembly configured to deliver two or more types of stimulation to a user. These modalities may include vibroacoustic stimulation, such as low-frequency vibrational energy; photonic stimulation, such as modulated light in the visible or near-infrared spectrum; and optionally transcranial alternating current stimulation (tACS), delivering phase-locked electrical signals through the scalp. This multi-layer assembly may be positioned adjacent to the cranial or cervical regions of the user to optimize therapeutic engagement.

The system further includes a craniofacial bone-conduction interface, which may comprise a frame or bar coupled to anatomical structures such as the maxilla, mandible, zygomatic arch, or mastoid. This interface includes vibroacoustic transducers or photonic emitters designed to transmit energy directly through bone, facilitating deep somatosensory or neurosensory stimulation.

One or more robotic actuators—such as multi-degree-of-freedom (e.g., 6-DOF) positioning arms—may be used to dynamically adjust the spatial orientation, contact pressure, and placement of the stimulation assembly relative to the user's anatomy. These robotic arms may incorporate force-feedback sensors to maintain optimal physical coupling during therapy.

The system incorporates a neural signal acquisition unit, such as a high-density EEG (HD-EEG) array, which captures real-time brainwave activity across multiple channels. This electrophysiological data is processed by a computing system configured with a personalization algorithm. The algorithm may leverage techniques such as reinforcement learning or Bayesian optimization to iteratively adapt stimulation parameters in response to user-specific EEG features and longitudinal behavioral outcomes.

The control interface allows for closed-loop modulation, whereby stimulation output is dynamically adjusted in real time based on the neural data acquired. In some embodiments, the vibroacoustic subsystem operates within a frequency range of approximately 20-300 Hz for bone-conducted resonance. The photonic subsystem may emit modulated light in the range of 400-900 nanometers, synchronized with vibratory harmonics. The tACS subsystem may deliver phase-locked current signals in the 1-40 Hz range, aligned to detected oscillatory phases.

Alternative embodiments support simplified systems where one or more stimulation modalities (vibroacoustic, photonic, or electromagnetic) are delivered via a mechanical interface coupled to the user's cranial, cervical, or facial region. Such interfaces may include intraoral devices, headbands, mandibular bars, or facial masks designed to ensure stable and comfortable delivery.

The stimulation may be informed by data captured from a neural monitoring unit, which may include consumer-grade or clinical EEG arrays. These signals are used by a processor executing a personalization algorithm to identify meaningful neural patterns and adjust therapy accordingly. A feedback loop enables real-time or iterative adjustments to maintain optimal neural engagement.

Specific configurations of the stimulation apparatus may include:
Vibroacoustic transducers delivering 20-300 Hz resonance.
Photonic emitters projecting 400-900 nm modulated light synchronized with vibration.
Electrode arrays delivering tACS or pulsed electromagnetic fields phase-aligned with endogenous brain rhythms.

Mechanically, these components may be coupled via craniofacial mounting structures that ensure optimal energy transfer and minimal displacement during use. The system may also incorporate cloud-based data transmission for remote updating of personalization models and synchronization across distributed devices.

The method of operating such a system may comprise:
Delivering neuromodulatory stimuli from among vibroacoustic, photonic, or electrical sources.
Maintaining mechanical contact with the cranial, facial, or cervical anatomy using a stimulation apparatus.
Acquiring neural data, such as EEG, in real time.
Processing these signals to extract features like oscillatory phase, power spectra, or synchrony.
Applying an adaptive algorithm—e.g., reinforcement learning or Bayesian inference—to compute updated stimulation parameters.
Adjusting stimulation in real time based on said computed parameters, thereby establishing a closed-loop neuromodulation system.

In some implementations, data is transmitted to a remote computing infrastructure or cloud-based repository, where it can be used to refine global models, generate predictive insights, or synchronize therapy profiles across different sessions and devices.

This architecture enables deeply personalized neuromodulation, adaptable over time and across contexts, and responsive to both neurophysiological input and therapeutic intent.

As used herein, a “closed acoustic circuit” denotes a bilateral vibroacoustic configuration in which anterior (upper) and posterior (lower) transducer arrays are driven with prescribed phase and gain to yield constructive interference and enhanced internal acoustic energy transfer through the torso relative to single-sided drive.

With the lower array fixed at 60-160 Hz carriers (0.2-0.5 g RMS at the array), an abdominal tri-axial accelerometer recorded internal acceleration. When the upper array was enabled with controller-set phase/gain, abdominal magnitude increased; when the upper array was deactivated while the lower drive remained constant, abdominal magnitude decreased markedly (optionally quantified in bench data as ≥10×; up to ≥50×), evidencing constructive interference and improved impedance matching.

The controller derives a per-user latency profile from photocell, microphone, accelerometer, or EEG markers and applies micro- to sub-second offsets to maintain cross-modal phase alignment; preferred windows include <200 ms (sleep/glymph), <100 ms (HRV/meditation), and <1 ms (ERP).
{“ts”:169.031,“node”:“latency_engine”,“rx_ms”:7.2,“proc_ms”:2.5,“tx_ms”:6.9,“end_to_end_ms”:16.6}

To target vasomotion, the controller drives two carriers f1, f2 within 40-200 Hz, synthesizing an envelope |f1−f2| in 0.05-0.5 Hz; envelopes may be swept or stepped and optionally synchronized to respiration or cardiovascular rhythms.

The controller may generate an independent therapeutic band and combine it with audio or other content at a summing node while monitoring accelerometer or microphone feedback to maintain intended amplitude and phase at a target region.

Closed-loop updates are computed from EEG that has been pre-processed to suppress stimulation-related artifacts (e.g., using reference accelerometers/microphones for subtraction) before extracting spectral targets (e.g., individualized gamma windows).

Thermal modules provide controlled heating/cooling synchronized with vibroacoustic drive. In certain embodiments, a predictive dual-exponential model is fit to surface/in-assembly temperature data; if predicted temperature exceeds a threshold, staged attenuation and hardware-level cutoff are triggered, with watchdog timers as a fail-safe.

Vestibular tilt/rock synchronized to ULF; camera-based OCR of external device displays; craniofacial/sternum “bar” with 2-4 transducers (magnetic attachment, offline/SD, back-to-sleep); retail/kiosk flow; additional pain-gating methods.

Human-subject data were de-identified per HIPAA/GDPR; only aggregated or pseudonymized metrics are disclosed.

### Closed Acoustic Circuit (Cross-Section, Two Panels)

Panel A: upper+lower arrays ON; arrows indicating constructive interference across torso; abdominal sensor vector magnitude indicated.
Panel B: upper OFF with lower constant; visibly reduced abdominal vector magnitude.

### Two-Carrier ULF Synthesis (Signal Traces)

Traces showing carrier f1, carrier f2, summed waveform, and extracted low-frequency envelope annotated with |f1−f2| (0.05-0.5 Hz).

1. An adaptive neuromodulation system comprising:
(a) a lower vibroacoustic transducer array configured to be positioned beneath a posterior body region of a user;
(b) an upper vibroacoustic transducer array configured to be positioned above an anterior body region of the user; and
(c) a control system operatively connected to the lower vibroacoustic transducer array and the upper vibroacoustic transducer array, the control system configured to
(i) drive the lower vibroacoustic transducer array and the upper vibroacoustic transducer array at one or more carrier frequencies between 5 Hz and 500 Hz, and
(ii) independently adjust phase and gain of the lower vibroacoustic transducer array and the upper vibroacoustic transducer array to produce constructive interference of vibroacoustic energy through a torso of the user, thereby forming a closed acoustic circuit through the body.

2. The device of claim 1, wherein deactivation of the upper vibroacoustic array while maintaining activation of the lower array produces a reduction in measured abdominal acceleration of at least ten-fold relative to bilateral operation.

3. The device of claim 1, wherein an over-body array is formed by a textile accessory selected from a weighted throw, cushion, or pillow insert, the accessory including a docking receptacle that retains a craniofacial or chestmounted transducer module to complete a closed acoustic circuit with an under-body array.

4. The system of claim 1, wherein the control system derives a per-user latency profile from sensed signals and applies phase, gain, or temporal offsets to maintain alignment.

5. The system of claim 4, wherein closed-loop latency is less than 200 ms for sleep/glymphatic programs, less than 100 ms for heart-rate-variability or meditation programs, and less than 1 ms for event-related-potential applications.

6. The system of claim 1, wherein the control system drives first and second carriers f1 and f2 within 20-300 Hz to synthesize a beat/envelope frequency |f1−f2| between 0.05 Hz and 0.5 Hz to target vasomotion-band dynamics, optionally steering the envelope spatially.

7. The system of claim 1, wherein the control system generates a first therapeutic frequency band, injects a second band independently, and combines the bands with sensor feedback to maintain intended amplitude and phase at a target region.

8-13. (canceled)

14. The system of claim 1, wherein over-body and/or perimeter modules cooperate with under-body transducers to create a three-dimensional vibrational field.

15. The system of claim 1, wherein redundant synchronization uses at least two of photocells, accelerometers, and microphones to measure timing and amplitude of vibroacoustic and audiovisual outputs across system elements.

16. A method of calibrating a vibroacoustic neuromodulation system comprising upper and lower vibroacoustic transducer arrays comprising:
positioning the lower array beneath a user;
positioning the upper array above the user;
driving the arrays at carrier frequencies between 5 Hz and 500 Hz;
adjusting phase and gain between the arrays to produce constructive interference;
measuring acceleration using one or more sensors; and
deriving and storing a per-user latency profile.

17. The method of claim 16, comprising driving carriers f1 and f2 to synthesize a low frequency envelope within 0.05-0.5 Hz, optionally sweeping the envelope over time and synchronizing to cardiovascular or respiratory rhythms.

18. The method of claim 16, further comprising generating a therapeutic band independent of audio content and combining the bands while maintaining target amplitude/phase via sensor feedback.

19. A non-transitory computer-readable medium storing instructions that, when executed by one or more processors, cause performance of the method of claim 16.

20. A neuromodulation system comprising at least one of robotic transducer placement, a craniofacial or over-body accessory, and two or more modalities, with sensor-based real-time control as in claim 1.

21. The system of claim 1, further comprising a pneumatic compression subsystem including a plurality of independently addressable inflatable aircell regions disposed in a layer adjacent to the lower vibroacoustic transducer array, the control system further configured to inflate and deflate the aircell regions in a phased sequence coordinated with the vibroacoustic stimulation to modulate mechanical coupling pressure between the transducer arrays and the body, and to promote lymphatic drainage and glymphatic flow through rhythmic compression of body regions.

22. The system of claim 21, wherein the phased sequence of aircell inflation and deflation is synchronized with a low-frequency envelope of the vibroacoustic stimulation such that peak mechanical compression coincides with a predetermined phase of the vibroacoustic carrier or envelope waveform.

23. The method of claim 16, further comprising: prompting the user to produce a sustained vocalization during vibroacoustic stimulation; recording an acoustic waveform of the vocalization via one or more microphones; computing spectral differences between the recorded waveform and a reference waveform obtained during a non-stimulation condition; and deriving from the spectral differences at least one of an internal resonance shift, a vibrational energy transmission depth, and phase or amplitude modulations in a natural resonance profile of the user, wherein the per-user latency profile incorporates data from both the acceleration measurement and the voice perturbation analysis.

24. The system of claim 4, wherein the control system stores the per-user latency profile in a non-volatile memory or remote database and, in a subsequent therapeutic session, retrieves the stored profile and applies the phase, gain, or temporal offsets derived therefrom to maintain cross-modal alignment without requiring a new calibration phase.

25. The system of claim 1, further comprising a synchronization interface configured to distribute a master timing reference to a plurality of neuromodulation nodes, each node comprising at least one vibroacoustic transducer array, and to apply node-level phase correction to maintain phase-coherent vibroacoustic outputs across the plurality of nodes during a simultaneous multi-user session.

26. The system of claim 1, further comprising a thermal management subsystem including one or more temperature sensors positioned at transducer interfaces, a predictive thermal model, and a hardware fail-safe comprising at least one of a watchdog timer and an analog cutoff relay, the thermal management subsystem configured to modulate transducer amplitude or duty cycle to maintain surface temperatures below a predefined safety threshold during sustained operation.

27. The system of claim 2, wherein the reduction in measured abdominal acceleration upon deactivation of the upper vibroacoustic array is at least fifty-fold relative to bilateral operation.

28. The system of claim 1, further comprising an electroencephalography sensor array, wherein the control system suppresses stimulation-related artifacts in signals from the electroencephalography sensor array using reference data from at least one of an accelerometer and a microphone, and extracts spectral features from the artifact-suppressed signals for closed-loop neuromodulation.

29. The system of claim 1, further comprising one or more visual stimulation modules selected from LED arrays, flicker glasses, a head-mounted optical interface, or an augmented reality or virtual reality display, the control system configured to synchronize pulsed or modulated visual stimuli with the vibroacoustic stimulation to achieve cross-modal phase coherence for multi-sensory entrainment.

30. The system of claim 1, wherein the lower vibroacoustic transducer array and the upper vibroacoustic transducer array collectively comprise a plurality of independently addressable transducer channels, and the control system is further configured to spatially pan vibroacoustic energy across the plurality of transducer channels to produce a perceived trajectory of stimulation moving through or across the body of the user.

31. The system of claim 1, wherein the control system implements a proportional-integral-derivative or adaptive control algorithm using feedback from array-mounted accelerometers to maintain vibration amplitude and phase coherence of the lower and upper vibroacoustic transducer arrays within defined tolerances during a therapeutic session.

32. The system of claim 24, wherein the stored per-user latency profile further incorporates voice perturbation calibration data derived from spectral analysis of the user's vocalization during vibroacoustic stimulation.

33. The method of claim 16, wherein measuring acceleration is performed using a portable handheld calibration device selected from a dedicated calibration instrument and a general-purpose mobile computing device executing calibration software, the device comprising at least an integrated accelerometer, an integrated microphone, and a display configured to render a real-time spectrogram of detected vibrations across time and frequency domains, the device repositionable by a practitioner or user to different anatomical zones of the user including above, below, and lateral to the user's body to measure at each zone localized vibrational amplitude, frequency distribution, waveform integrity, harmonic content, and vibrational penetration depth, and wherein the device further supports a voice perturbation diagnostic mode in which the user produces a sustained vocalization during vibroacoustic stimulation and the device records spectral differences between vibrational and non-vibrational conditions to infer energy coupling to internal tissues.

34. The system of claim 1, further comprising a kiosk architecture configured for high-throughput individual neuromodulatory profiling, the kiosk comprising: a high-density electroencephalography sensor array; one or more spatial imaging cameras configured to capture three-dimensional head and body geometry of the user for electrode alignment and anatomical registration; physiological sensors for concurrent biometric data acquisition; and a control interface configured to capture successive neuromap snapshots at defined intervals, analyze frequency-band power shifts, latency variations, and coherence metrics across the snapshots, generate an individualized change report, and apply the change report to tailor subsequent vibroacoustic stimulation protocols to the user's neural response profile.

35. The system of claim 1, further comprising a craniofacial bone-conduction bar mechanically coupled to at least one cranial or mandibular surface of the user, the bar comprising an embedded vibration transducer and contact sensors, and one or more robotic actuators or positioning arms providing multi-degree-of-freedom spatial orientation and force-feedback control to maintain calibrated contact pressure against the craniofacial surface, the control system configured to deliver vibroacoustic energy through bone conduction pathways of the user's cranial structures in synchronization with the lower and upper vibroacoustic transducer arrays.

36. The system of claim 4, wherein the control system further comprises a behavioral psychophysics and gamma optimization subsystem configured to: generate synchronized multimodal stimuli across auditory, visual, and vibroacoustic channels from a common timing reference; sweep vibroacoustic stimulation frequencies across a gamma frequency range to identify the user's individualized peak gamma resonance frequency; acquire concurrent neural data from an electroencephalography sensor and behavioral response data from user interactions; apply an adaptive optimization algorithm to refine stimulation parameters based on frequency-dependent entrainment efficiency; and generate and store a personalized gamma map specifying the user's peak gamma resonance frequency, preferred amplitude ratios across modalities, and adaptive thresholds, the personalized gamma map retrieved in subsequent sessions to initialize stimulation parameters near the user's empirically validated resonance zone.

37. The system of claim 36, wherein the adaptive optimization algorithm further identifies theta-gamma coupling metrics from the electroencephalography data and adjusts the vibroacoustic stimulation to enhance cross-frequency coupling between theta-band and gamma-band neural oscillations to support memory consolidation.

38. The system of claim 1, further comprising an electroencephalography sensor, wherein the control system monitors alpha-band power in signals from the electroencephalography sensor during vibroacoustic stimulation and applies conditional modulation of stimulation parameters based on detected changes in alpha-band synchrony, wherein an increase in alpha-band power above a predefined threshold triggers a reduction in stimulation intensity or a shift in stimulation frequency.

39. The system of claim 1, further comprising one or more eye-tracking sensors configured to monitor at least one of gaze position, gaze dynamics, pupil diameter, and pupil response of the user during vibroacoustic stimulation, the control system configured to receive eye-tracking data from the one or more eye-tracking sensors and to adjust at least one of frequency, amplitude, phase, or spatial targeting of the vibroacoustic stimulation based on detected changes in gaze dynamics or pupil response indicative of a change in the user's cognitive or autonomic state.

40. A vibroacoustic neuromodulation apparatus comprising:
(a) a flexible vibroacoustic transducer array comprising one or more vibroacoustic transducers embedded within flexible substrates or textile layers and integrated into an accessory selected from a wrap, cushion, or wearable garment configured to couple to a body region of a user;
(b) a power source enabling autonomous operation of the apparatus independent of a base platform; and
(c) a controller configured to
(i) drive the one or more vibroacoustic transducers at a first carrier frequency and a second carrier frequency, each between about 20 Hz and 300 Hz; and
(ii) synthesize a low-frequency envelope from the first and second carrier frequencies, the envelope having a frequency between about 0.05 Hz and 0.5 Hz.

41. The apparatus of claim 8, wherein the controller is further configured to vary the envelope frequency during a therapeutic session by sweeping or stepping the difference between the first carrier frequency and the second carrier frequency.

42. The apparatus of claim 8, wherein the controller synchronizes the envelope frequency with at least one physiological rhythm of the user selected from respiration and cardiovascular activity.

43. The apparatus of claim 8, wherein the vibroacoustic transducer array is integrated into a textile accessory selected from a wrap, blanket, cushion, or wearable garment configured to conform to a torso region of the user.

44. The apparatus of claim 8, further comprising one or more physiological sensors configured to provide feedback signals to the controller for adaptive modulation of vibroacoustic stimulation.

45. The apparatus of claim 8, further comprising a wireless communication interface configured to exchange user profile data or stimulation parameters with a remote control platform or cloud-based system.
